SEQUENCE LISTING (134 sequences)

SEQ ID NO: 1 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 3 - protein, 7 aa, synthetic construct
  REGION 1..7: note = Synthetic
PKKKRKV

SEQ ID NO: 4 - protein, 7 aa, synthetic construct
  REGION 1..7: note = Synthetic
PKKKRRV

SEQ ID NO: 5 - protein, 16 aa, synthetic construct
  REGION 1..16: note = Synthetic
KRPAATKKAGQAKKKK

SEQ ID NO: 6 - RNA, 16 nt, synthetic construct
  misc_feature 1..16: note = Synthetic
  source 1..16: mol_type = other RNA
gttttagagctatgct

SEQ ID NO: 7 - RNA, 22 nt, synthetic construct
  misc_feature 1..22: note = Synthetic
  source 1..22: mol_type = other RNA
gttttagagctatgctgttttg

SEQ ID NO: 8 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 9 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 10 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 11 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 12 - RNA, 82 nt, synthetic construct; identical to SEQ ID NO: 10
  misc_feature 1..82: note = Synthetic
  source 1..82: mol_type = other RNA

SEQ ID NO: 13 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 14 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 15 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 16 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 17 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 18 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 19-21 - skipped (3 entries)

SEQ ID NO: 22 - DNA, 3198 bp, Mus musculus
  source 1..3198: mol_type = other DNA
gtgtccggggcggggcggtcccggggcggggcccggagcgggctgcggttgcggtccctgcgccggcggtgaaggcgcagcagcggcgagtggctattgcaagcgttcggataatgtgagacctggaatgcagtgagacctgggatgcagggatgtcgactatctgccccccaccatctcctgctgttgccaagacagagattgctttaagtggtgaatcacccttgttggcggctacctttgcttactgggataatattcttggtcctagagtaaggcatatttgggctccaaagacagaccaagtgcttctcagtgatggagaaataacttttcttgccaaccacactctaaatggagaaattcttcgaaatgcagagagtggggctatagatgtaaaattttttgtcttatctgaaaaaggggtaattattgtttcattaatcttcgacggaaactggaatggagatcggagcacttatggactatcaattatactgccgcagacagagctgagcttctacctcccacttcacagagtgtgtgttgacaggctaacacacattattcgaaaaggaagaatatggatgcataaggaaagacaagaaaatgtccagaaaattgtcttggaaggcacagagaggatggaagatcagggtcagagtatcattcccatgcttactggggaagtcattcctgtaatggagctgcttgcatctatgaaatcccacagtgttcctgaagacattgatatagctgatacagtgctcaatgatgatgacattggtgacagctgtcacgaaggctttcttctcaatgccatcagctcacacctgcagacctgtggctgttccgttgtagttggcagcagtgcagagaaagtaaataagatagtaagaacgctgtgcctttttctgacaccagcagagaggaaatgctccaggctgtgtgaagcagaatcgtcctttaagtacgaatcgggactctttgtgcaaggcttgctaaaggatgcaacaggcagttttgtcctacccttccggcaagttatgtatgccccgtaccccaccacgcacattgatgtggatgtcaacactgtcaagcagatgccaccgtgtcatgaacatatttataatcaacgcagatacatgaggtcagagctgacagccttctggagggcaacttcagaagaggacatggcgcaggacaccatcatctacacagatgagagcttcactcctgatttgaatattttccaagatgtcttacacagagacactctagtgaaagccttcctggatcaggtcttccatttgaagcctggcctgtctctcaggagtactttccttgcacagttcctcctcattcttcacagaaaagccttgacactaatcaagtacatcgaggatgatacgcagaaggggaaaaagccctttaagtctcttcggaacctgaagatagatcttgatttaacagcagagggcgatcttaacataataatggctctagctgagaaaattaagccaggcctacactctttcatctttgggagacctttctacactagtgtacaagaacgtgatgttctaatgaccttttgaccgtgtggtttgctgtgtctgtctcttcacagtcacacctgctgttacagtgtctcagcagtgtgtgggcacatccttcctcccgagtcctgctgcaggacagggtacactacacttgtcagtagaagtctgtacctgatgtcaggtgcatcgttacagtgaatgactcttcctagaatagatgtactcttttagggccttatgtttacaattatcctaagtactattgctgtcttttaaagatatgaatgatggaatatacacttgaccataactgctgattggttttttgttttgttttgtttgttttcttggaaacttatgattcctggtttacatgtaccacactgaaaccctcgttagctttacagataaagtgtgagttgacttcctgcccctctgtgttctgtggtatgtccgattacttctgccacagctaaacattagagcatttaaagtttgcagttcctcagaaaggaacttagtctgactacagattagttcttgagagaagacactgatagggcagagctgtaggtgaaatcagttgttagcccttcctttatagacgtagtccttcagattcggtctgtacagaaatgccgaggggtcatgcatgggccctgagtatcgtgacctgtgacaagttttttgttggtttattgtagttctgtcaaagaaagtggcatttgtttttataattgttgccaacttttaaggttaattttcattatttttgagccgaattaaaatgcgcacctcctgtgcctttcccaatcttggaaaatataatttcttggcagagggtcagatttcagggcccagtcactttcatctgaccaccctttgcacggctgccgtgtgcctggcttagattagaagtccttgttaagtatgtcagagtacattcgctgataagatctttgaagagcagggaagcgtcttgcctctttcctttggtttctgcctgtactctggtgtttcccgtgtcacctgcatcataggaacagcagagaaatctgacccagtgctatttttctaggtgctactatggcaaactcaagtggtctgtttctgttcctgtaacgttcgactatctcgctagctgtgaagtactgattagtggagttctgtgcaacagcagtgtaggagtatacacaaacacaaatatgtgtttctatttaaaactgtggacttagcataaaaagggagaatatatttattttttacaaaagggataaaaatgggccccgttcctcacccaccagatttagcgagaaaaagctttctattctgaaaggtcacggtggctttggcattacaaatcagaacaacacacactgaccatgatggcttgtgaactaactgcaaggcactccgtcatggtaagcgagtaggtcccacctcctagtgtgccgctcattgctttacacagtagaatcttatttgagtgctaattgttgtctttgctgctttactgtgttgttatagaaaatgtaagctgtacagtgaataagttattgaagcatgtgtaaacactgttatatatcttttctcctagatggggaattttgaataaaatacctttgaaattctgtgt

SEQ ID NO: 23 - protein, 481 aa, Mus musculus
MSTICPPPSPAVAKTEIALSGESPLLAATFAYWDNILGPRVRHIWAPKTDQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIVLEGTERMEDQGQSIIPMLTGEVIPVMELLASMKSHSVPEDIDIADTVLNDDDIGDSCHEGFLLNAISSHLQTCGCSVVVGSSAEKVNKIVRTLCLFLTPAERKCSRLCEAESSFKYESGLFVQGLLKDATGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFHLKPGLSLRSTFLAQFLLILHRKALTLIKYIEDDTQKGKKPFKSLRNLKIDLDLTAEGDLNIIMALAEKIKPGLHSFIFGRPFYTSVQERDVLMTF

SEQ ID NO: 24 - DNA, 3435 bp, Rattus norvegicus
  source 1..3435: mol_type = other DNA
cgtttgtagtgtcagccatcccaattgcctgttccttctctgtgggagtggtgtctagacagtccaggcagggtatgctaggcaggtgcgttttggttgcctcagatcgcaacttgactccataacggtgaccaaagacaaaagaaggaaaccagattaaaaagaaccggacacagacccctgcagaatctggagcggccgtggttgggggcggggctacgacggggcggactcgggggcgtgggagggcggggccggggcggggcccggagccggctgcggttgcggtccctgcgccggcggtgaaggcgcagcggcggcgagtggctattgcaagcgtttggataatgtgagacctgggatgcagggatgtcgactatctgccccccaccatctcctgctgttgccaagacagagattgctttaagtggtgaatcacccttgttggcggctacctttgcttactgggataatattcttggtcctagagtaaggcacatttgggctccaaagacagaccaagtactcctcagtgatggagaaatcacttttcttgccaaccacactctgaatggagaaattcttcggaatgcggagagtggggcaatagatgtaaagttttttgtcttatctgaaaagggcgtcattattgtttcattaatcttcgacgggaactggaacggagatcggagcacttacggactatcaattatactgccgcagacggagctgagtttctacctcccactgcacagagtgtgtgttgacaggctaacgcacatcattcgaaaaggaaggatatggatgcacaaggaaagacaagaaaatgtccagaaaattgtcttggaaggcaccgagaggatggaagatcagggtcagagtatcatccctatgcttactggggaggtcatccctgtgatggagctgcttgcgtctatgagatcacacagtgttcctgaagacctcgatatagctgatacagtactcaatgatgatgacattggtgacagctgtcatgaaggctttcttctcaatgccatcagctcacatctgcagacctgcggctgttctgtggtggtaggcagcagtgcagagaaagtaaataagatagtaagaacactgtgcctttttctgacaccagcagagaggaagtgctccaggctgtgtgaagccgaatcgtcctttaaatacgaatctggactctttgtacaaggcttgctaaaggatgcgactggcagttttgtactacctttccggcaagttatgtatgccccttatcccaccacacacatcgatgtggatgtcaacactgtcaagcagatgccaccgtgtcatgaacatatttataatcaacgcagatacatgaggtcagagctgacagccttctggagggcaacttcagaagaggacatggctcaggacaccatcatctacacagatgagagcttcactcctgatttgaatattttccaagatgtcttacacagagacactctagtgaaagcctttctggatcaggtcttccatttgaagcctggcctgtctctcaggagtactttccttgcacagttcctcctcattcttcacagaaaagccttgacactaatcaagtacatagaggatgacacgcagaaggggaaaaagccctttaagtctcttcggaacctgaagatagatcttgatttaacagcagagggcgaccttaacataataatggctctagctgagaaaattaagccaggcctacactctttcatcttcgggagacctttctacactagtgtccaagaacgtgatgttctaatgactttttaaacatgtggtttgctccgtgtgtctcatgacagtcacacttgctgttacagtgtctcagcgctttggacacatccttcctccagggtcctgccgcaggacacgttacactacacttgtcagtagaggtctgtaccagatgtcaggtacatcgttgtagtgaatgtctcttttcctagactagatgtaccctcgtagggacttatgtttacaaccctcctaagtactagtgctgtcttgtaaggatacgaatgaagggatgtaaacttcaccacaactgctggttggttttgttgtttttgttttttgaaacttataattcatggtttacatgcatcacactgaaaccctagttagctttttacaggtaagctgtgagttgactgcctgtccctgtgttctctggcctgtacgatctgtggcgtgtaggatcacttttgcaacaactaaaaactaaagcactttgtttgcagttctacagaaagcaacttagtctgtctgcagattcgtttttgaaagaagacatgagaaagcggagttttaggtgaagtcagttgttggatcttcctttatagacttagtcctttagatgtggtctgtatagacatgcccaaccatcatgcatgggcactgaatatcgtgaactgtggtatgctttttgttggtttattgtacttctgtcaaagaaagtggcattggtttttataattgttgccaagttttaaggttaattttcattatttttgagccaaattaaaatgtgcacctcctgtgcctttcccaatcttggaaaatataatttcttggcagaaggtcagatttcagggcccagtcactttcgtctgacttccctttgcacagtccgccatgggcctggcttagaagttcttgtaaactatgccagagagtacattcgctgataaaatcttctttgcagagcaggagagcttcttgcctctttcctttcatttctgcctggactttggtgttctccacgttccctgcatcctaaggacagcaggagaactctgaccccagtgctatttctctaggtgctattgtggcaaactcaagcggtccgtctctgtccctgtaacgttcgtaccttgctggctgtgaagtactgactggtaaagctccgtgctacagcagtgtagggtatacacaaacacaagtaagtgttttatttaaaactgtggacttagcataaaaagggagactatatttattttttacaaaagggataaaaatggaaccctttcctcacccaccagatttagtcagaaaaaaacattctattctgaaaggtcacagtggttttgacatgacacatcagaacaacgcacactgtccatgatggcttatgaactccaagtcactccatcatggtaaatgggtagatccctccttctagtgtgccacaccattgcttcccacagtagaatcttatttaagtgctaagtgttgtctctgctggtttactctgttgttttagagaatgtaagttgtatagtgaataagttattgaagcatgtgtaaacactgttatacatcttttctcctagatggggaatttggaataaaatacctttaaaattcaaaaaaaaaaaaaaaaaaaaaaaa

SEQ ID NO: 25 - protein, 481 aa, Rattus norvegicus
MSTICPPPSPAVAKTEIALSGESPLLAATFAYWDNILGPRVRHIWAPKTDQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIVLEGTERMEDQGQSIIPMLTGEVIPVMELLASMRSHSVPEDLDIADTVLNDDDIGDSCHEGFLLNAISSHLQTCGCSVVVGSSAEKVNKIVRTLCLFLTPAERKCSRLCEAESSFKYESGLFVQGLLKDATGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFHLKPGLSLRSTFLAQFLLILHRKALTLIKYIEDDTQKGKKPFKSLRNLKIDLDLTAEGDLNIIMALAEKIKPGLHSFIFGRPFYTSVQERDVLMTF

SEQ ID NO: 26 - DNA, 1957 bp, Homo sapiens
  source 1..1957: mol_type = other DNA
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttataaaaaaaa

SEQ ID NO: 27 - protein, 222 aa, Homo sapiens
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLK

SEQ ID NO: 28 - DNA, 3261 bp, Homo sapiens
  source 1..3261: mol_type = other DNA
gggcggggctgcggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttaaaaaaaaaaaaaaaaaa

SEQ ID NO: 29 - protein, 481 aa, Homo sapiens
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLNAISSHLQTCGCSVVVGSSAEKVNKIVRTLCLFLTPAERKCSRLCEAESSFKYESGLFVQGLLKDSTGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFQLKPGLSLRSTFLAQFLLVLHRKALTLIKYIEDDTQKGKKPFKSLRNLKIDLDLTAEGDLNIIMALAEKIKPGLHSFIFGRPFYTSVQERDVLMTF

SEQ ID NO: 30 - DNA, 3356 bp, Homo sapiens
  source 1..3356: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttaaaaaaaaaaaaaaaaaa

SEQ ID NO: 31 - protein, 481 aa, Homo sapiens; identical to SEQ ID NO: 29

SEQ ID NO: 32 - DNA, 20 bp, synthetic construct
  misc_feature 1..20: note = Synthetic
  source 1..20: mol_type = other DNA
ctttccttctaggtggaaag

SEQ ID NO: 33 - DNA, 20 bp, synthetic construct
  misc_feature 1..20: note = Synthetic
  source 1..20: mol_type = other DNA
cgtctctgcttgacgcgccg

SEQ ID NO: 34 - DNA, 20 bp, synthetic construct
  misc_feature 1..20: note = Synthetic
  source 1..20: mol_type = other DNA
aagcgccaacccggaactta

SEQ ID NO: 35 - DNA, 20 bp, synthetic construct
  misc_feature 1..20: note = Synthetic
  source 1..20: mol_type = other DNA
agcccatgacagcctccgcc

SEQ ID NO: 36 - DNA, 20 bp, synthetic construct
  misc_feature 1..20: note = Synthetic
  source 1..20: mol_type = other DNA
gtaacctagatagataacgc

SEQ ID NO: 37 - DNA, 20 bp, synthetic construct
  misc_feature 1..20: note = Synthetic
  source 1..20: mol_type = other DNA
acggcatttaaccgtgtcat

SEQ ID NO: 38 - DNA, 20 bp, synthetic construct
  misc_feature 1..20: note = Synthetic
  source 1..20: mol_type = other DNA
aacacggtgactacgcacag

SEQ ID NO: 39 - DNA, 20 bp, synthetic construct
  misc_feature 1..20: note = Synthetic
  source 1..20: mol_type = other DNA
aatcaataaatcagacgtca

SEQ ID NO: 40 - DNA, 20 bp, synthetic construct
  misc_feature 1..20: note = Synthetic
  source 1..20: mol_type = other DNA
agttctatttttaccgaagg

SEQ ID NO: 41 - DNA, 20 bp, synthetic construct
  misc_feature 1..20: note = Synthetic
  source 1..20: mol_type = other DNA
tcttacagtgttttgtaccg

SEQ ID NO: 42 - DNA, 20 bp, synthetic construct
  misc_feature 1..20: note = Synthetic
  source 1..20: mol_type = other DNA
aaatagcataggaccatgtt

SEQ ID NO: 43 - DNA, 20 bp, synthetic construct
  misc_feature 1..20: note = Synthetic
  source 1..20: mol_type = other DNA
gtcagctaatccacaacacg

SEQ ID NO: 44 - DNA, 20 bp, synthetic construct
  misc_feature 1..20: note = Synthetic
  source 1..20: mol_type = other DNA
atcaattacattgatagctc

SEQ ID NO: 45 - DNA, 20 bp, synthetic construct
  misc_feature 1..20: note = Synthetic
  source 1..20: mol_type = other DNA
tgttagtttgactcacagac

SEQ ID NO: 46 - DNA, 20 bp, synthetic construct
  misc_feature 1..20: note = Synthetic
  source 1..20: mol_type = other DNA
gtctctaggtaaaattttga

SEQ ID NO: 47 - DNA, 20 bp, synthetic construct
  misc_feature 1..20: note = Synthetic
  source 1..20: mol_type = other DNA
ctaaaatattgtatgaccac

SEQ ID NO: 48 - DNA, 20 bp, synthetic construct
  misc_feature 1..20: note = Synthetic
  source 1..20: mol_type = other DNA
tggaacaattacttaggcat

SEQ ID NO: 49 - DNA, 20 bp, synthetic construct
  misc_feature 1..20: note = Synthetic
  source 1..20: mol_type = other DNA
taattgttgtattgcctgaa

SEQ ID NO: 50 - DNA, 20 bp, synthetic construct
  misc_feature 1..20: note = Synthetic
  source 1..20: mol_type = other DNA
aatctacctcaaataactcc

SEQ ID NO: 51 - DNA, 20 bp, synthetic construct
  misc_feature 1..20: note = Synthetic
  source 1..20: mol_type = other DNA
ggagtaaaccagtacgtggt

SEQ ID NO: 52 - DNA, 20 bp, synthetic construct
  misc_feature 1..20: note = Synthetic
  source 1..20: mol_type = other DNA
tgcgatgacgttttctcacg

SEQ ID NO: 53 - DNA, 20 bp, synthetic construct
  misc_feature 1..20: note = Synthetic
  source 1..20: mol_type = other DNA
ctcacgaggctagcgaaatg

SEQ ID NO: 54 - DNA, 20 bp, synthetic construct
  misc_feature 1..20: note = Synthetic
  source 1..20: mol_type = other DNA
gcgccaacgctcctccagag

SEQ ID NO: 55 - DNA, 20 bp, synthetic construct
  misc_feature 1..20: note = Synthetic
  source 1..20: mol_type = other DNA
tgtgcgaaccttaatagggg

SEQ ID NO: 56 - DNA, 20 bp, synthetic construct
  misc_feature 1..20: note = Synthetic
  source 1..20: mol_type = other DNA
ggcgctcaacacataattgg

SEQ ID NO: 57 - DNA, 20 bp, synthetic construct
  misc_feature 1..20: note = Synthetic
  source 1..20: mol_type = other DNA
accctggtaggggacagctc

SEQ ID NO: 58 - DNA, 20 bp, synthetic construct
  misc_feature 1..20: note = Synthetic
  source 1..20: mol_type = other DNA
caagcatgattttcacactg

SEQ ID NO: 59 - DNA, 20 bp, synthetic construct
  misc_feature 1..20: note = Synthetic
  source 1..20: mol_type = other DNA
cctgcagaccaaaagacgca

SEQ ID NO: 60 - DNA, 20 bp, synthetic construct
  misc_feature 1..20: note = Synthetic
  source 1..20: mol_type = other DNA
tttgatctgcaagtgtgaga

SEQ ID NO: 61 - DNA, 20 bp, synthetic construct
  misc_feature 1..20: note = Synthetic
  source 1..20: mol_type = other DNA
aacgttttaatcattcaccg

SEQ ID NO: 62 - DNA, 20 bp, synthetic construct
  misc_feature 1..20: note = Synthetic
  source 1..20: mol_type = other DNA
ttcacaaagcatagaacagt

SEQ ID NO: 63 - DNA, 20 bp, synthetic construct
  misc_feature 1..20: note = Synthetic
  source 1..20: mol_type = other DNA
atacccaactaaaatagtgg

SEQ ID NO: 64 - DNA, 20 bp, synthetic construct
  misc_feature 1..20: note = Synthetic
  source 1..20: mol_type = other DNA
acttggagagaattacactg

SEQ ID NO: 65 - DNA, 20 bp, synthetic construct
  misc_feature 1..20: note = Synthetic
  source 1..20: mol_type = other DNA
ttggcactattaaggatctg

SEQ ID NO: 66 - DNA, 20 bp, synthetic construct
  misc_feature 1..20: note = Synthetic
  source 1..20: mol_type = other DNA
gggcatacatgtagatcaga

SEQ ID NO: 67 - DNA, 20 bp, synthetic construct
  misc_feature 1..20: note = Synthetic
  source 1..20: mol_type = other DNA
agttgtacttcacttatacg

SEQ ID NO: 68 - DNA, 20 bp, synthetic construct
  misc_feature 1..20: note = Synthetic
  source 1..20: mol_type = other DNA
cagagtagacccttggttgg

SEQ ID NO: 69 - DNA, 20 bp, synthetic construct
  misc_feature 1..20: note = Synthetic
  source 1..20: mol_type = other DNA
gggagaccccagacttcaca

SEQ ID NO: 70 - DNA, 20 bp, synthetic construct
  misc_feature 1..20: note = Synthetic
  source 1..20: mol_type = other DNA
tggctttcagacttgcaatg

SEQ ID NO: 71 - DNA, 20 bp, synthetic construct
  misc_feature 1..20: note = Synthetic
  source 1..20: mol_type = other DNA
tctcctttgactcacaccca

SEQ ID NO: 72 - RNA, 20 nt, synthetic construct
  misc_feature 1..20: note = Synthetic
  source 1..20: mol_type = other RNA
ctttccttctaggtggaaag

SEQ ID NO: 73 - RNA, 20 nt, synthetic construct
  misc_feature 1..20: note = Synthetic
  source 1..20: mol_type = other RNA
cgtctctgcttgacgcgccg

SEQ ID NO: 74 - RNA, 20 nt, synthetic construct
  misc_feature 1..20: note = Synthetic
  source 1..20: mol_type = other RNA
aagcgccaacccggaactta

SEQ ID NO: 75 - RNA, 20 nt, synthetic construct
  misc_feature 1..20: note = Synthetic
  source 1..20: mol_type = other RNA
agcccatgacagcctccgcc

SEQ ID NO: 76 - RNA, 20 nt, synthetic construct
  misc_feature 1..20: note = Synthetic
  source 1..20: mol_type = other RNA
gtaacctagatagataacgc

SEQ ID NO: 77 - RNA, 20 nt, synthetic construct
  misc_feature 1..20: note = Synthetic
  source 1..20: mol_type = other RNA
acggcatttaaccgtgtcat

SEQ ID NO: 78 - RNA, 20 nt, synthetic construct
  misc_feature 1..20: note = Synthetic
  source 1..20: mol_type = other RNA
aacacggtgactacgcacag

SEQ ID NO: 79 - RNA, 20 nt, synthetic construct
  misc_feature 1..20: note = Synthetic
  source 1..20: mol_type = other RNA
aatcaataaatcagacgtca

SEQ ID NO: 80 - RNA, 20 nt, synthetic construct
  misc_feature 1..20: note = Synthetic
  source 1..20: mol_type = other RNA
agttctatttttaccgaagg

SEQ ID NO: 81 - RNA, 20 nt, synthetic construct
  misc_feature 1..20: note = Synthetic
  source 1..20: mol_type = other RNA
tcttacagtgttttgtaccg

SEQ ID NO: 82 - RNA, 20 nt, synthetic construct
  misc_feature 1..20: note = Synthetic
  source 1..20: mol_type = other RNA
aaatagcataggaccatgtt

SEQ ID NO: 83 - RNA, 20 nt, synthetic construct
  misc_feature 1..20: note = Synthetic
  source 1..20: mol_type = other RNA
gtcagctaatccacaacacg

SEQ ID NO: 84 - RNA, 20 nt, synthetic construct
  misc_feature 1..20: note = Synthetic
  source 1..20: mol_type = other RNA
atcaattacattgatagctc

SEQ ID NO: 85 - RNA, 20 nt, synthetic construct
  misc_feature 1..20: note = Synthetic
  source 1..20: mol_type = other RNA
tgttagtttgactcacagac

SEQ ID NO: 86 - RNA, 20 nt, synthetic construct
  misc_feature 1..20: note = Synthetic
  source 1..20: mol_type = other RNA
gtctctaggtaaaattttga

SEQ ID NO: 87 - RNA, 20 nt, synthetic construct
  misc_feature 1..20: note = Synthetic
  source 1..20: mol_type = other RNA
ctaaaatattgtatgaccac

SEQ ID NO: 88 - RNA, 20 nt, synthetic construct
  misc_feature 1..20: note = Synthetic
  source 1..20: mol_type = other RNA
tggaacaattacttaggcat

SEQ ID NO: 89 - RNA, 20 nt, synthetic construct
  misc_feature 1..20: note = Synthetic
  source 1..20: mol_type = other RNA
taattgttgtattgcctgaa

SEQ ID NO: 90 - RNA, 20 nt, synthetic construct
  misc_feature 1..20: note = Synthetic
  source 1..20: mol_type = other RNA
aatctacctcaaataactcc

SEQ ID NO: 91 - RNA, 20 nt, synthetic construct
  misc_feature 1..20: note = Synthetic
  source 1..20: mol_type = other RNA
ggagtaaaccagtacgtggt

SEQ ID NO: 92 - RNA, 20 nt, synthetic construct
  misc_feature 1..20: note = Synthetic
  source 1..20: mol_type = other RNA
tgcgatgacgttttctcacg

SEQ ID NO: 93 - RNA, 20 nt, synthetic construct
  misc_feature 1..20: note = Synthetic
  source 1..20: mol_type = other RNA
ctcacgaggctagcgaaatg

SEQ ID NO: 94 - RNA, 20 nt, synthetic construct
  misc_feature 1..20: note = Synthetic
  source 1..20: mol_type = other RNA
gcgccaacgctcctccagag

SEQ ID NO: 95 - RNA, 20 nt, synthetic construct
  misc_feature 1..20: note = Synthetic
  source 1..20: mol_type = other RNA
tgtgcgaaccttaatagggg

SEQ ID NO: 96 - RNA, 20 nt, synthetic construct
  misc_feature 1..20: note = Synthetic
  source 1..20: mol_type = other RNA
ggcgctcaacacataattgg

SEQ ID NO: 97 - RNA, 20 nt, synthetic construct
  misc_feature 1..20: note = Synthetic
  source 1..20: mol_type = other RNA
accctggtaggggacagctc

SEQ ID NO: 98 - RNA, 20 nt, synthetic construct
  misc_feature 1..20: note = Synthetic
  source 1..20: mol_type = other RNA
caagcatgattttcacactg

SEQ ID NO: 99 - RNA, 20 nt, synthetic construct
  misc_feature 1..20: note = Synthetic
  source 1..20: mol_type = other RNA
cctgcagaccaaaagacgca

SEQ ID NO: 100 - RNA, 20 nt, synthetic construct
  misc_feature 1..20: note = Synthetic
  source 1..20: mol_type = other RNA
tttgatctgcaagtgtgaga

SEQ ID NO: 101 - RNA, 20 nt, synthetic construct
  misc_feature 1..20: note = Synthetic
  source 1..20: mol_type = other RNA
aacgttttaatcattcaccg

SEQ ID NO: 102 - RNA, 20 nt, synthetic construct
  misc_feature 1..20: note = Synthetic
  source 1..20: mol_type = other RNA
ttcacaaagcatagaacagt

SEQ ID NO: 103 - RNA, 20 nt, synthetic construct
  misc_feature 1..20: note = Synthetic
  source 1..20: mol_type = other RNA
atacccaactaaaatagtgg

SEQ ID NO: 104 - RNA, 20 nt, synthetic construct
  misc_feature 1..20: note = Synthetic
  source 1..20: mol_type = other RNA
acttggagagaattacactg

SEQ ID NO: 105 - RNA, 20 nt, synthetic construct
  misc_feature 1..20: note = Synthetic
  source 1..20: mol_type = other RNA
ttggcactattaaggatctg

SEQ ID NO: 106 - RNA, 20 nt, synthetic construct
  misc_feature 1..20: note = Synthetic
  source 1..20: mol_type = other RNA
gggcatacatgtagatcaga

SEQ ID NO: 107 - RNA, 20 nt, synthetic construct
  misc_feature 1..20: note = Synthetic
  source 1..20: mol_type = other RNA
agttgtacttcacttatacg

SEQ ID NO: 108 - RNA, 20 nt, synthetic construct
  misc_feature 1..20: note = Synthetic
  source 1..20: mol_type = other RNA
cagagtagacccttggttgg

SEQ ID NO: 109 - RNA, 20 nt, synthetic construct
  misc_feature 1..20: note = Synthetic
  source 1..20: mol_type = other RNA
gggagaccccagacttcaca

SEQ ID NO: 110 - RNA, 20 nt, synthetic construct
  misc_feature 1..20: note = Synthetic
  source 1..20: mol_type = other RNA
tggctttcagacttgcaatg

SEQ ID NO: 111 - RNA, 20 nt, synthetic construct
  misc_feature 1..20: note = Synthetic
  source 1..20: mol_type = other RNA
tctcctttgactcacaccca

SEQ ID NO: 112 - DNA, 20 bp, synthetic construct
  misc_feature 1..20: note = Synthetic
  source 1..20: mol_type = other DNA
aaacagacagacgtaaccta

SEQ ID NO: 113 - RNA, 20 nt, synthetic construct
  misc_feature 1..20: note = Synthetic
  source 1..20: mol_type = other RNA
aaacagacagacgtaaccta

SEQ ID NO: 114 - DNA, 20 bp, synthetic construct
  misc_feature 1..20: note = Synthetic
  source 1..20: mol_type = other DNA
gccggggccggggccggggc

SEQ ID NO: 115 - DNA, 20 bp, synthetic construct
  misc_feature 1..20: note = Synthetic
  source 1..20: mol_type = other DNA
ccggggccggggccggggcc

SEQ ID NO: 116 - DNA, 20 bp, synthetic construct
  misc_feature 1..20: note = Synthetic
  source 1..20: mol_type = other DNA
cggggccggggccggggccg

SEQ ID NO: 117 - DNA, 20 bp, synthetic construct
  misc_feature 1..20: note = Synthetic
  source 1..20: mol_type = other DNA
cccggccccggccccggccc

SEQ ID NO: 118 - RNA, 20 nt, synthetic construct
  misc_feature 1..20: note = Synthetic
  source 1..20: mol_type = other RNA
gccggggccggggccggggc

SEQ ID NO: 119 - RNA, 20 nt, synthetic construct
  misc_feature 1..20: note = Synthetic
  source 1..20: mol_type = other RNA
ccggggccggggccggggcc

SEQ ID NO: 120 - RNA, 20 nt, synthetic construct
  misc_feature 1..20: note = Synthetic
  source 1..20: mol_type = other RNA
cggggccggggccggggccg

SEQ ID NO: 121 - RNA, 20 nt, synthetic construct
  misc_feature 1..20: note = Synthetic
  source 1..20: mol_type = other RNA
cccggccccggccccggccc

SEQ ID NO: 122 - DNA, 20 bp, synthetic construct
  misc_feature 1..20: note = Synthetic
  source 1..20: mol_type = other DNA
gagcaggtgtgggtttagga

SEQ ID NO: 123 - DNA, 20 bp, synthetic construct
  misc_feature 1..20: note = Synthetic
  source 1..20: mol_type = other DNA
ccaggtctcactgcattcca

SEQ ID NO: 124 - DNA, 26 bp, synthetic construct
  misc_feature 1..26: note = Synthetic
  source 1..26: mol_type = other DNA
attgcaagcgttcggataatgtgaga

SEQ ID NO: 125 - DNA, 18 bp, synthetic construct
  misc_feature 1..18: note = Synthetic
  source 1..18: mol_type = other DNA
ggtggcgagtggctattg

SEQ ID NO: 126 - DNA, 21 bp, synthetic construct
  misc_feature 1..21: note = Synthetic
  source 1..21: mol_type = other DNA
gatagtcgacatccctgcatc

SEQ ID NO: 127 - DNA, 26 bp, synthetic construct
  misc_feature 1..26: note = Synthetic
  source 1..26: mol_type = other DNA
aagcgttcggataatgtgagacctgg

SEQ ID NO: 128 - DNA, 20 bp, synthetic construct
  misc_feature 1..20: note = Synthetic
  source 1..20: mol_type = other DNA
aagagcaggtgtgggtttag

SEQ ID NO: 129 - DNA, 19 bp, synthetic construct
  misc_feature 1..19: note = Synthetic
  source 1..19: mol_type = other DNA
cggttgtttccctccttgt

SEQ ID NO: 130 - DNA, 24 bp, synthetic construct
  misc_feature 1..24: note = Synthetic
  source 1..24: mol_type = other DNA
tctcacagtactcgctgagggtga

SEQ ID NO: 131 - DNA, 20 bp, synthetic construct
  misc_feature 1..20: note = Synthetic
  source 1..20: mol_type = other DNA
cccactacttgctctcacag

SEQ ID NO: 132 - DNA, 18 bp, synthetic construct
  misc_feature 1..18: note = Synthetic
  source 1..18: mol_type = other DNA
tacaggctgcggttgttt

SEQ ID NO: 133 - DNA, 23 bp, synthetic construct
  misc_feature 1..23: note = Synthetic
  source 1..23: mol_type = other DNA
actcgctgagggtgaacaagaaa

SEQ ID NO: 134 - DNA, 20 bp, synthetic construct
  misc_feature 1..20: note = Synthetic
  source 1..20: mol_type = other DNA
gcatttttgatgaagcaaga

(19) United States
(12) Patent Application Publication
(10) Pub. No.: US 2025/0381303 A1
(43) Pub. Date: Dec. 18, 2025

(54) CRISPR INTERFERENCE THERAPEUTICS FOR C9ORF72 REPEAT EXPANSION DISEASE

(71) Applicant: Regeneron Pharmaceuticals, Inc., Tarrytown, NY (US)

(72) Inventors: Sriramkumar Sundaramoorthy, New York, NY (US); David Frendewey, New York, NY (US)

(73) Assignee: Regeneron Pharmaceuticals, Inc., Tarrytown, NY (US)

(21) Appl. No.: 18/867,568

(22) PCT Filed: May 31, 2023
(86) PCT No.: PCT/US2023/067655

§ 371 Date: Nov. 20, 2024

Related U.S. Application Data
(60) Provisional application No. 63/365,558, filed on May 31, 2022.

(51) Int. Cl. A61K 48/00; A61K 9/00; A61P 25/28; C12N 9/22; C12N 15/11; C12N 15/86; C12N 15/88

(52) U.S. Cl. CPC A61K 48/0058; A61K 9/0048; A61K 48/0075; A61P 25/28; C12N 9/226; C12N 15/111; C12N 15/86; C12N 15/88; C12N 2310/20; C12N 2310/315; C12N 2310/321; C12N 2750/14143

(57) ABSTRACT

Guide RNAs and CRISPR/Cas systems targeting a C9orf72 gene, lipid nanoparticles or viral vectors comprising such CRISPR/Cas systems, and cells or animals comprising such CRISPR/Cas systems are provided. Methods of repressing transcription from a C9orf72 exon 1 A transcription start site and/or repressing transcription of sense and/or antisense transcripts that comprise the hexanucleotide repeat expansion sequence in a C9orf72 gene using the CRISPR/Cas systems are also provided, as well as use of the CRISPR/Cas systems in prophylactic and therapeutic applications for treatment and/or prevention of a C9orf72 hexanucleotide repeat expansion associated disease and/or for ameliorating at least one symptom associated with such disease.

### CROSS-REFERENCE TO RELATED APPLICATIONS

This application claims the benefit of U.S. Application No. 63/365,558, filed May 31, 2022, which is herein incorporated by reference in its entirety for all purposes.

### REFERENCE TO A SEQUENCE LISTING SUBMITTED AS AN XML FILE VIA EFS WEB

The Sequence Listing written in file 057766-596446.xml is 180 kilobytes, was created on May 30, 2023, and is hereby incorporated by reference.

### BACKGROUND

Amyotrophic lateral sclerosis (ALS) and frontotemporal lobar degeneration (FTLD) are progressive and fatal neurodegenerative diseases that cause motor neuron disease in the case of ALS and dementia in the case of FTLD. The most common cause of familial ALS is an expansion of a GGGGCC (G₄C₂) hexanucleotide repeat between two alternative 5′ non-coding exons of the C9orf72 gene. Normal individuals have between 3 and 35 G₄C₂ repeats, while ALS or FTLD patients harbor repeat numbers in the hundreds or thousands. The physiological function of the C9orf72 protein is not well understood, and no disease-causing mutations have been identified in its coding sequence. There are no effective cures currently available.

### SUMMARY

Provided are guide RNAs and CRISPR/Cas systems targeting a C9orf72 gene, lipid nanoparticles or viral vectors comprising such CRISPR/Cas systems, and cells or animals comprising such CRISPR/Cas systems. Also provided are methods of repressing transcription from a C9orf72 exon 1A transcription start site and/or repressing transcription of sense and/or antisense transcripts that comprise the hexanucleotide repeat expansion sequence in a C9orf72 gene, as well as use of the CRISPR/Cas systems in prophylactic and therapeutic applications for treatment and/or prevention of a C9orf72 hexanucleotide repeat expansion associated disease and/or for ameliorating at least one symptom associated with such disease.

In one aspect, provided are methods of repressing transcription from a C9orf72 exon 1A transcription start site or repressing transcription of sense or antisense transcripts that comprise the hexanucleotide repeat expansion sequence in a C9orf72 gene in a cell. Some such methods comprise contacting the C9orf72 gene with a first CRISPR/Cas complex comprising a nuclease-inactive Cas protein and a first guide RNA comprising a first DNA-targeting segment that targets a first guide RNA target sequence upstream of or proximate to the C9orf72 exon 1A transcription start site, wherein the first CRISPR/Cas complex binds to the first guide RNA target sequence. Some such methods comprise contacting the C9orf72 gene with a second CRISPR/Cas complex comprising the nuclease-inactive Cas protein and a second guide RNA comprising a second DNA-targeting segment that targets a second guide RNA target sequence within a C9orf72 hexanucleotide repeat expansion sequence between the first non-coding endogenous exon and exon 2 of the C9orf72 gene, wherein the second CRISPR/Cas complex binds to the second guide RNA target sequence. Some such methods comprise: (a) contacting the C9orf72 gene with a first CRISPR/Cas complex comprising a nuclease-inactive Cas protein and a first guide RNA comprising a first DNA-targeting segment that targets a first guide RNA target sequence upstream of or proximate to the C9orf72 exon 1A transcription start site, wherein the first CRISPR/Cas complex binds to the first guide RNA target sequence; and/or (b) contacting the C9orf72 gene with a second CRISPR/Cas complex comprising the nuclease-inactive Cas protein and a second guide RNA comprising a second DNA-targeting segment that targets a second guide RNA target sequence within a C9orf72 hexanucleotide repeat expansion sequence between the first non-coding endogenous exon and exon 2 of the C9orf72 gene, wherein the second CRISPR/Cas complex binds to the first guide RNA target sequence. In some such methods, the method comprises option (a). In some such methods, the method comprises option (b). In some such methods, the method comprises options (a) and (b).

In some such methods, option (a) comprises contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site, and/or wherein option (b) comprises contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene. In some such methods, option (a) comprises contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site, and/or wherein option (b) comprises contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene.

In some such methods, the C9orf72 hexanucleotide repeat expansion sequence has more than 30, more than 100, more than 200, more than 300, more than 400, or more than 500 repeats of the hexanucleotide sequence G₄C₂.

In some such methods, the first guide RNA target sequence is within 250, within 225, within 200, within 175, within 150, within 125, within 100, within 75, or within 50 nucleotides of the C9orf72 exon 1A transcription start site. In some such methods, the first guide RNA target sequence is within 100, within 75, or within 50 nucleotides of the C9orf72 exon 1A transcription start site.

In some such methods, the nuclease-inactive Cas protein is not fused to a heterologous transcriptional repressor domain, and the guide RNA is not linked to a heterologous transcriptional repressor domain.

In some such methods, the binding reduces or abolishes expression of transcripts that initiate at C9orf72 exon 1A. In some such methods, the binding reduces or abolishes expression of transcripts that initiate at C9orf72 exon 1A but does not reduce or abolish expression of transcripts that initiate at C9orf72 exon 1B. In some such methods, the binding reduces or abolishes expression of C9orf72 hexanucleotide-repeat-containing transcripts. In some such methods, the binding reduces or abolishes expression of C9orf72 hexanucleotide-repeat-containing transcripts but does not reduce or abolish expression of transcripts that initiate at C9orf72 exon 1B. In some such methods, the binding reduces or abolishes expression of both sense and antisense C9orf72 hexanucleotide-repeat-containing transcripts. In some such methods, the binding reduces or abolishes expression of both sense and antisense C9orf72 hexanucleotide-repeat-containing transcripts but does not reduce or abolish expression of transcripts that initiate at C9orf72 exon 1B.

In some such methods, the method comprises: (a) introducing the nuclease-inactive Cas protein or a nucleic acid encoding the nuclease-inactive Cas protein and the first guide RNA or one or more DNAs encoding the first guide RNA into the cell; and/or (b) introducing the nuclease-inactive Cas protein or the nucleic acid encoding the nuclease-inactive Cas protein and the second guide RNA or one or more DNAs encoding the second guide RNA into the cell.

In some such methods, the first guide RNA is a single guide RNA (sgRNA). In some such methods, the nuclease-inactive Cas protein is a nuclease-inactive Cas9 protein. In some such methods, the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the nuclease-inactive Cas protein is derived from a Streptococcus pyogenes Cas9 protein. In some such methods, the nucleic acid encoding the nuclease-inactive Cas protein is codon-optimized for expression in a mammalian cell or a human cell. In some such methods, the method comprises: (a) introducing the first guide RNA in the form of RNA, optionally wherein the first guide RNA comprises at least one modification; and/or (b) introducing the second guide RNA in the form of RNA, optionally wherein the second guide RNA comprises at least one modification. In some such methods, the at least one modification comprises a 2′-O-methyl-modified nucleotide and/or a phosphorothioate bond between nucleotides. In some such methods, the method comprises introducing the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification. In some such methods, the method comprises: (a) introducing the nucleic acid encoding the nuclease-inactive Cas protein and the one or more DNAs encoding the first guide RNA, wherein the nucleic acid encoding the nuclease-inactive Cas protein comprises DNA; and/or (b) introducing the nucleic acid encoding the nuclease-inactive Cas protein and the one or more DNAs encoding the second guide RNA, wherein the nucleic acid encoding the nuclease-inactive Cas protein comprises DNA. In some such methods, (a) the DNA encoding the nuclease-inactive Cas protein and the one or more DNAs encoding the first guide RNA are in one or more vectors; and/or (b) the DNA encoding the nuclease-inactive Cas protein and the one or more DNAs encoding the second guide RNA are in one or more vectors. In some such methods, the one or more vectors are one or more viral vectors. In some such methods, the one or more viral vectors are one or more adeno-associated virus (AAV) vectors. In some such methods, (a) the nuclease-inactive Cas protein or the nucleic acid encoding the nuclease-inactive Cas protein and the first guide RNA or the one or more DNAs encoding the first guide RNA are associated with a lipid nanoparticle; and/or (b) the nuclease-inactive Cas protein or the nucleic acid encoding the nuclease-inactive Cas protein and the second guide RNA or the one or more DNAs encoding the second guide RNA are associated with a lipid nanoparticle.

In some such methods, the first DNA-targeting segment comprises at least 17, at least 18, at least 19, or at least 20 contiguous nucleotides of the sequence set forth in any one of SEQ ID NOS: 72-111 and 113. In some such methods, the first DNA-targeting segment is at least 90% or at least 95% identical to the sequence set forth in any one of SEQ ID NOS: 72-111 and 113. In some such methods, the first guide RNA target sequence comprises at least 17, at least 18, at least 19, or at least 20 contiguous nucleotides of the sequence set forth in any one of SEQ ID NOS: 32-71 and 112. In some such methods, the first DNA-targeting segment comprises at least 17, at least 18, at least 19, or at least 20 contiguous nucleotides of the sequence set forth in any one of SEQ ID NOS: 93-95. In some such methods, the first DNA-targeting segment is at least 90% or at least 95% identical to the sequence set forth in any one of SEQ ID NOS: 93-95. In some such methods, the first guide RNA target sequence comprises at least 17, at least 18, at least 19, or at least 20 contiguous nucleotides of the sequence set forth in any one of SEQ ID NOS: 53-55. In some such methods, the first DNA-targeting segment comprises at least 17, at least 18, at least 19, or at least 20 contiguous nucleotides of the sequence set forth in SEQ ID NO: 74. In some such methods, the first DNA-targeting segment is at least 90% or at least 95% identical to the sequence set forth in SEQ ID NO: 74. In some such methods, the first guide RNA target sequence comprises at least 17, at least 18, at least 19, or at least 20 contiguous nucleotides of the sequence set forth in SEQ ID NO: 34. In some such methods, the second DNA-targeting segment comprises at least 17, at least 18, at least 19, or at least 20 contiguous nucleotides of the sequence set forth in any one of SEQ ID NOS: 118-121. In some such methods, the second DNA-targeting segment is at least 90% or at least 95% identical to the sequence set forth in any one of SEQ ID NOS: 118-121. In some such methods, the second guide RNA target sequence comprises at least 17, at least 18, at least 19, or at least 20 contiguous nucleotides of the sequence set forth in any one of SEQ ID NOS: 114-117. In some such methods, the second DNA-targeting segment comprises at least 17, at least 18, at least 19, or at least 20 contiguous nucleotides of the sequence set forth in SEQ ID NO: 74. In some such methods, the second DNA-targeting segment is at least 90% or at least 95% identical to the sequence set forth in SEQ ID NO: 74. In some such methods, the second guide RNA target sequence comprises at least 17, at least 18, at least 19, or at least 20 contiguous nucleotides of the sequence set forth in SEQ ID NO: 34.

In some such methods, the cell is a neuron, optionally wherein the neuron is a motor neuron. In some such methods, the cell is in vitro or ex vivo. In some such methods, the cell is in a subject in vivo, optionally wherein the subject is a human. In some such methods, the cell is a neuron in the brain of the subject. In some such methods, the subject has or is at risk for developing a C9orf72 hexanucleotide repeat expansion associated disease. In some such methods, the C9orf72 hexanucleotide repeat expansion associated disease is amyotrophic lateral sclerosis (ALS) or frontotemporal dementia (FTD). In some such methods, (a) the nuclease-inactive Cas protein or a nucleic acid encoding the nuclease-inactive Cas protein and the first guide RNA or one or more DNAs encoding the first guide RNA are administered to the subject by intracerebroventricular injection, intracranial injection, or intrathecal injection; and/or (b) the nuclease-inactive Cas protein or a nucleic acid encoding the nuclease-inactive Cas protein and the second guide RNA or one or more DNAs encoding the second guide RNA are administered to the subject by intracerebroventricular injection, intracranial injection, or intrathecal injection. In some such methods, the cell is a mammalian cell, and the C9orf72 gene is a mammalian C9orf72 gene. In some such methods, the cell is a human cell. In some such methods, the cell is a mouse cell. In some such methods, the C9orf72 gene comprises a human C9orf72 promoter. In some such methods, the C9orf72 gene is a human C9orf72 gene or a humanized C9orf72 gene.

In another aspect, provided are methods of repressing transcription from a C9orf72 exon 1A transcription start site or repressing transcription of sense or antisense transcripts that comprise the hexanucleotide repeat expansion sequence in a C9orf72 gene in a subject. Some such methods comprise: (a) administering to the subject a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or (b) administering to the subject the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence. In another aspect, provided are methods of preventing, treating, or ameliorating at least one symptom or indication of a C9orf72 hexanucleotide repeat expansion associated disease. Some such methods comprise: (a) administering to a subject in need thereof a first pharmaceutical composition comprising a therapeutically effective amount of a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or (b) administering to the subject in need thereof a second pharmaceutical composition comprising a therapeutically effective amount of the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence. In some such methods, the C9orf72 hexanucleotide repeat expansion associated disease is amyotrophic lateral sclerosis (ALS) or frontotemporal dementia (FTD). In some such methods, the method comprises option (a). In some such methods, the method comprises option (b). In some such methods, the method comprises options (a) and (b).

In some such methods, option (a) comprises administering to the subject at least two guide RNAs, wherein each guide RNA guide RNA targets a different guide RNA target sequence upstream of or proximate to the C9orf72 exon 1A transcription start site, and/or wherein option (b) comprises administering to the subject at least two guide RNAs, wherein each guide RNA guide RNA targets a different guide RNA target sequence within the C9orf72 hexanucleotide repeat expansion sequence between the first non-coding endogenous exon and exon 2 of the C9orf72 gene. In some such methods, option (a) comprises administering to the subject at least three guide RNAs, wherein each guide RNA guide RNA targets a different guide RNA target sequence upstream of or proximate to the C9orf72 exon 1A transcription start site, and/or wherein option (b) comprises administering to the subject at least three guide RNAs, wherein each guide RNA guide RNA targets a different guide RNA target sequence within the C9orf72 hexanucleotide repeat expansion sequence between the first non-coding endogenous exon and exon 2 of the C9orf72 gene.

In some such methods, the C9orf72 hexanucleotide repeat expansion sequence has more than 30, more than 100, more than 200, more than 300, more than 400, or more than 500 repeats of the hexanucleotide sequence G₄C₂. In some such methods, the administering is intracerebroventricular injection, intracranial injection, or intrathecal injection.

In some such methods, the first guide RNA target sequence is within 250, within 225, within 200, within 175, within 150, within 125, within 100, within 75, or within 50 nucleotides of the C9orf72 exon 1A transcription start site. In some such methods, the first guide RNA target sequence is within 100, within 75, or within 50 nucleotides of the C9orf72 exon 1A transcription start site.

In some such methods, the nuclease-inactive Cas protein is not fused to a heterologous transcriptional repressor domain, and the guide RNA is not linked to a heterologous transcriptional repressor domain.

In some such methods, the binding reduces or abolishes expression of transcripts that initiate at C9orf72 exon 1A. In some such methods, the binding reduces or abolishes expression of transcripts that initiate at C9orf72 exon 1A but does not reduce or abolish expression of transcripts that initiate at C9orf72 exon 1B. In some such methods, the binding reduces or abolishes expression of C9orf72 hexanucleotide-repeat-containing transcripts. In some such methods, the binding reduces or abolishes expression of C9orf72 hexanucleotide-repeat-containing transcripts but does not reduce or abolish expression of transcripts that initiate at C9orf72 exon 1B. In some such methods, the binding reduces or abolishes expression of both sense and antisense C9orf72 hexanucleotide-repeat-containing transcripts. In some such methods, the binding reduces or abolishes expression of both sense and antisense C9orf72 hexanucleotide-repeat-containing transcripts but does not reduce or abolish expression of transcripts that initiate at C9orf72 exon 1B.

In some such methods, the first guide RNA is a single guide RNA (sgRNA). In some such methods, the nuclease-inactive Cas protein is a nuclease-inactive Cas9 protein. In some such methods, the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the nuclease-inactive Cas protein is derived from a Streptococcus pyogenes Cas9 protein. In some such methods, the nucleic acid encoding the nuclease-inactive Cas protein is codon-optimized for expression in a mammalian cell or a human cell. In some such methods, the method comprises: (a) administering the first guide RNA in the form of RNA, optionally wherein the first guide RNA comprises at least one modification; and/or (b) administering the second guide RNA in the form of RNA, optionally wherein the second guide RNA comprises at least one modification. In some such methods, the at least one modification comprises a 2′-O-methyl-modified nucleotide and/or a phosphorothioate bond between nucleotides. In some such methods, the method comprises administering the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification. In some such methods, the method comprises: (a) administering the nucleic acid encoding the nuclease-inactive Cas protein and the one or more DNAs encoding the first guide RNA, wherein the nucleic acid encoding the nuclease-inactive Cas protein comprises DNA; and/or (b) administering the nucleic acid encoding the nuclease-inactive Cas protein and the one or more DNAs encoding the second guide RNA, wherein the nucleic acid encoding the nuclease-inactive Cas protein comprises DNA. In some such methods, (a) the DNA encoding the nuclease-inactive Cas protein and the one or more DNAs encoding the first guide RNA are in one or more vectors; and/or (b) the DNA encoding the nuclease-inactive Cas protein and the one or more DNAs encoding the second guide RNA are in one or more vectors. In some such methods, the one or more vectors are one or more viral vectors. In some such methods, the one or more viral vectors are one or more adeno-associated virus (AAV) vectors. In some such methods, (a) the nuclease-inactive Cas protein or the nucleic acid encoding the nuclease-inactive Cas protein and the first guide RNA or the one or more DNAs encoding the first guide RNA are associated with a lipid nanoparticle; and/or (b) the nuclease-inactive Cas protein or the nucleic acid encoding the nuclease-inactive Cas protein and the second guide RNA or the one or more DNAs encoding the second guide RNA are associated with a lipid nanoparticle.

In some such methods, the first DNA-targeting segment comprises at least 17, at least 18, at least 19, or at least 20 contiguous nucleotides of the sequence set forth in any one of SEQ ID NOS: 72-111 and 113. In some such methods, the first DNA-targeting segment is at least 90% or at least 95% identical to the sequence set forth in any one of SEQ ID NOS: 72-111 and 113. In some such methods, the first guide RNA target sequence comprises at least 17, at least 18, at least 19, or at least 20 contiguous nucleotides of the sequence set forth in any one of SEQ ID NOS: 32-71 and 112. In some such methods, the first DNA-targeting segment comprises at least 17, at least 18, at least 19, or at least 20 contiguous nucleotides of the sequence set forth in any one of SEQ ID NOS: 93-95. In some such methods, the first DNA-targeting segment is at least 90% or at least 95% identical to the sequence set forth in any one of SEQ ID NOS: 93-95. In some such methods, the first guide RNA target sequence comprises at least 17, at least 18, at least 19, or at least 20 contiguous nucleotides of the sequence set forth in any one of SEQ ID NOS: 53-55. In some such methods, the first DNA-targeting segment comprises at least 17, at least 18, at least 19, or at least 20 contiguous nucleotides of the sequence set forth in SEQ ID NO: 74. In some such methods, the first DNA-targeting segment is at least 90% or at least 95% identical to the sequence set forth in SEQ ID NO: 74. In some such methods, the first guide RNA target sequence comprises at least 17, at least 18, at least 19, or at least 20 contiguous nucleotides of the sequence set forth in SEQ ID NO: 34. In some such methods, the second DNA-targeting segment comprises at least 17, at least 18, at least 19, or at least 20 contiguous nucleotides of the sequence set forth in any one of SEQ ID NOS: 118-121. In some such methods, the second DNA-targeting segment is at least 90% or at least 95% identical to the sequence set forth in any one of SEQ ID NOS: 118-121. In some such methods, the second guide RNA target sequence comprises at least 17, at least 18, at least 19, or at least 20 contiguous nucleotides of the sequence set forth in any one of SEQ ID NOS: 114-117. In some such methods, the second DNA-targeting segment comprises at least 17, at least 18, at least 19, or at least 20 contiguous nucleotides of the sequence set forth in SEQ ID NO: 74. In some such methods, the second DNA-targeting segment is at least 90% or at least 95% identical to the sequence set forth in SEQ ID NO: 74. In some such methods, the second guide RNA target sequence comprises at least 17, at least 18, at least 19, or at least 20 contiguous nucleotides of the sequence set forth in SEQ ID NO: 34.

In some such methods, the binding is in neurons in the subject, optionally wherein the neurons are motor neurons. In some such methods, the neurons are in the brain of the subject. In some such methods, the subject is a mammalian subject, and the C9orf72 gene is a mammalian C9orf72 gene. In some such methods, the subject is a human subject. In some such methods, the subject is a mouse subject. In some such methods, the C9orf72 gene comprises a human C9orf72 promoter. In some such methods, the C9orf72 gene is a human C9orf72 gene or a humanized C9orf72 gene.

In another aspect, provided are CRISPR/Cas systems. Some such systems comprise: (a) a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or (b) the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence. In some such systems, the CRISPR/Cas system comprises option (a). In some such systems, the CRISPR/Cas system comprises option (b). In some such systems, the CRISPR/Cas system comprises options (a) and (b).

In some such systems, option (a) comprises at least two guide RNAs, wherein each guide RNA targets a different guide RNA target sequence upstream of or proximate to the C9orf72 exon 1A transcription start site, and/or option (b) comprises at least two guide RNAs, wherein each guide RNA targets a different guide RNA target sequence within the C9orf72 hexanucleotide repeat expansion sequence between the first non-coding endogenous exon and exon 2 of the C9orf72 gene. In some such systems, option (a) comprises at least three guide RNAs, wherein each guide RNA targets a different guide RNA target sequence upstream of or proximate to the C9orf72 exon 1A transcription start site, and/or option (b) comprises at least three guide RNAs, wherein each guide RNA targets a different guide RNA target sequence within the C9orf72 hexanucleotide repeat expansion sequence between the first non-coding endogenous exon and exon 2 of the C9orf72 gene.

In some such systems, the first guide RNA target sequence is within 250, within 225, within 200, within 175, within 150, within 125, within 100, within 75, or within 50 nucleotides of the C9orf72 exon 1A transcription start site. In some such systems, the first guide RNA target sequence is within 100, within 75, or within 50 nucleotides of the C9orf72 exon 1A transcription start site.

In some such systems, the nuclease-inactive Cas protein is not fused to a heterologous transcriptional repressor domain, and the guide RNA is not linked to a heterologous transcriptional repressor domain.

In some such systems, the binding reduces or abolishes expression of transcripts that initiate at C9orf72 exon 1A. In some such systems, the binding reduces or abolishes expression of transcripts that initiate at C9orf72 exon 1A but does not reduce or abolish expression of transcripts that initiate at C9orf72 exon 1B. In some such systems, the binding reduces or abolishes expression of C9orf72 hexanucleotide-repeat-containing transcripts. In some such systems, the binding reduces or abolishes expression of C9orf72 hexanucleotide-repeat-containing transcripts but does not reduce or abolish expression of transcripts that initiate at C9orf72 exon 1B. In some such systems, the binding reduces or abolishes expression of both sense and antisense C9orf72 hexanucleotide-repeat-containing transcripts. In some such systems, the binding reduces or abolishes expression of both sense and antisense C9orf72 hexanucleotide-repeat-containing transcripts but does not reduce or abolish expression of transcripts that initiate at C9orf72 exon 1B.

In some such systems, the first guide RNA is a single guide RNA (sgRNA). In some such systems, the nuclease-inactive Cas protein is a nuclease-inactive Cas9 protein. In some such systems, the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such systems, the nuclease-inactive Cas protein is derived from a Streptococcus pyogenes Cas9 protein. In some such systems, the nucleic acid encoding the nuclease-inactive Cas protein is codon-optimized for expression in a mammalian cell or a human cell. In some such systems, the CRISPR/Cas system comprises: (a) the first guide RNA in the form of RNA, optionally wherein the first guide RNA comprises at least one modification; and/or (b) the second guide RNA in the form of RNA, optionally wherein the second guide RNA comprises at least one modification. In some such systems, the at least one modification comprises a 2′-O-methyl-modified nucleotide and/or a phosphorothioate bond between nucleotides. In some such systems, the CRISPR/Cas system comprises the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification. In some such systems, the CRISPR/Cas system comprises: (a) the nucleic acid encoding the nuclease-inactive Cas protein and the one or more DNAs encoding the first guide RNA, wherein the nucleic acid encoding the nuclease-inactive Cas protein comprises DNA; and/or (b) the nucleic acid encoding the nuclease-inactive Cas protein and the one or more DNAs encoding the second guide RNA, wherein the nucleic acid encoding the nuclease-inactive Cas protein comprises DNA. In some such systems, (a) the DNA encoding the nuclease-inactive Cas protein and the one or more DNAs encoding the first guide RNA are in one or more vectors; and/or (b) the DNA encoding the nuclease-inactive Cas protein and the one or more DNAs encoding the second guide RNA are in one or more vectors. In some such systems, the one or more vectors are one or more viral vectors. In some such systems, the one or more viral vectors are one or more adeno-associated virus (AAV) vectors. In some such systems, (a) the nuclease-inactive Cas protein or the nucleic acid encoding the nuclease-inactive Cas protein and the first guide RNA or the one or more DNAs encoding the first guide RNA are associated with a lipid nanoparticle; and/or (b) the nuclease-inactive Cas protein or the nucleic acid encoding the nuclease-inactive Cas protein and the second guide RNA or the one or more DNAs encoding the second guide RNA are associated with a lipid nanoparticle.

In some such systems, the first DNA-targeting segment comprises at least 17, at least 18, at least 19, or at least 20 contiguous nucleotides of the sequence set forth in any one of SEQ ID NOS: 72-111 and 113. In some such systems, the first DNA-targeting segment is at least 90% or at least 95% identical to the sequence set forth in any one of SEQ ID NOS: 72-111 and 113. In some such systems, the first guide RNA target sequence comprises at least 17, at least 18, at least 19, or at least 20 contiguous nucleotides of the sequence set forth in any one of SEQ ID NOS: 32-71 and 112. In some such systems, the first DNA-targeting segment comprises at least 17, at least 18, at least 19, or at least 20 contiguous nucleotides of the sequence set forth in any one of SEQ ID NOS: 93-95. In some such systems, the first DNA-targeting segment is at least 90% or at least 95% identical to the sequence set forth in any one of SEQ ID NOS: 93-95. In some such systems, the first guide RNA target sequence comprises at least 17, at least 18, at least 19, or at least 20 contiguous nucleotides of the sequence set forth in any one of SEQ ID NOS: 53-55. In some such systems, the first DNA-targeting segment comprises at least 17, at least 18, at least 19, or at least 20 contiguous nucleotides of the sequence set forth in SEQ ID NO: 74. In some such systems, the first DNA-targeting segment is at least 90% or at least 95% identical to the sequence set forth in SEQ ID NO: 74. In some such systems, the first guide RNA target sequence comprises at least 17, at least 18, at least 19, or at least 20 contiguous nucleotides of the sequence set forth in SEQ ID NO: 34. In some such systems, the second DNA-targeting segment comprises at least 17, at least 18, at least 19, or at least 20 contiguous nucleotides of the sequence set forth in any one of SEQ ID NOS: 118-121. In some such systems, the second DNA-targeting segment is at least 90% or at least 95% identical to the sequence set forth in any one of SEQ ID NOS: 118-121. In some such systems, the second guide RNA target sequence comprises at least 17, at least 18, at least 19, or at least 20 contiguous nucleotides of the sequence set forth in any one of SEQ ID NOS: 114-117. In some such systems, the second DNA-targeting segment comprises at least 17, at least 18, at least 19, or at least 20 contiguous nucleotides of the sequence set forth in SEQ ID NO: 74. In some such systems, the second DNA-targeting segment is at least 90% or at least 95% identical to the sequence set forth in SEQ ID NO: 74. In some such systems, the second guide RNA target sequence comprises at least 17, at least 18, at least 19, or at least 20 contiguous nucleotides of the sequence set forth in SEQ ID NO: 34.

In some such systems, the C9orf72 gene is a mammalian C9orf72 gene. In some such systems, the C9orf72 gene comprises a human C9orf72 promoter. In some such systems, the C9orf72 gene is a human C9orf72 gene or a humanized C9orf72 gene.

In another aspect, provide are pharmaceutical compositions comprising any of the above CRISPR/Cas systems and a pharmaceutically acceptable carrier.

In another aspect, provided are compositions comprising a guide RNA or one or more DNAs encoding the guide RNA. In some such compositions, the guide RNA comprises a DNA-targeting segment that targets a guide RNA target sequence in a C9orf72 gene, wherein the guide RNA target sequence is within a C9orf72 hexanucleotide repeat expansion sequence between the first non-coding endogenous exon and exon 2 of the C9orf72 gene, and wherein the guide RNA can bind to a nuclease-inactive Cas protein and target the nuclease-inactive Cas protein to the guide RNA target sequence.

In some such compositions, the binding of the Cas protein to the guide RNA target sequence reduces or abolishes expression of transcripts that initiate at C9orf72 exon 1A. In some such compositions, binding of the Cas protein to the guide RNA target sequence reduces or abolishes expression of transcripts that initiate at C9orf72 exon 1A but does not reduce or abolish expression of transcripts that initiate at C9orf72 exon 1B. In some such compositions, binding of the Cas protein to the guide RNA target sequence reduces or abolishes expression of C9orf72 hexanucleotide-repeat-containing transcripts. In some such compositions, binding of the Cas protein to the guide RNA target sequence reduces or abolishes expression of C9orf72 hexanucleotide-repeat-containing transcripts but does not reduce or abolish expression of transcripts that initiate at C9orf72 exon 1B. In some such compositions, binding of the Cas protein to the guide RNA target sequence reduces or abolishes expression of both sense and antisense C9orf72 hexanucleotide-repeat-containing transcripts. In some such compositions, binding of the Cas protein to the guide RNA target sequence reduces or abolishes expression of both sense and antisense C9orf72 hexanucleotide-repeat-containing transcripts but does not reduce or abolish expression of transcripts that initiate at C9orf72 exon 1B.

In some such compositions, the guide RNA is a single guide RNA (sgRNA). In some such compositions, the nuclease-inactive Cas protein is a nuclease-inactive Cas9 protein. In some such compositions, the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such compositions, the nuclease-inactive Cas protein is derived from a Streptococcus pyogenes Cas9 protein. In some such compositions, the CRISPR/Cas system comprises the guide RNA in the form of RNA, optionally wherein the guide RNA comprises at least one modification. In some such compositions, the at least one modification comprises a 2′-O-methyl-modified nucleotide and/or a phosphorothioate bond between nucleotides. In some such compositions, the one or more DNAs encoding the guide RNA are in one or more vectors. In some such compositions, the one or more vectors are one or more viral vectors. In some such compositions, the one or more viral vectors are one or more adeno-associated virus (AAV) vectors. In some such compositions, the guide RNA or the one or more DNAs encoding the guide RNA are associated with a lipid nanoparticle.

In some such compositions, the DNA-targeting segment comprises at least 17, at least 18, at least 19, or at least 20 contiguous nucleotides of the sequence set forth in any one of SEQ ID NOS: 118-121. In some such compositions, the DNA-targeting segment is at least 90% or at least 95% identical to the sequence set forth in any one of SEQ ID NOS: 118-121. In some such compositions, the guide RNA target sequence comprises at least 17, at least 18, at least 19, or at least 20 contiguous nucleotides of the sequence set forth in any one of SEQ ID NOS: 114-117.

In some such compositions, the C9orf72 gene is a mammalian C9orf72 gene. In some such compositions, the C9orf72 gene comprises a human C9orf72 promoter. In some such compositions, the C9orf72 gene is a human C9orf72 gene or a humanized C9orf72 gene.

In another aspect, provided are pharmaceutical compositions comprising any of the above compositions and a pharmaceutically acceptable carrier.

### BRIEF DESCRIPTION OF THE FIGURES

FIG. 1 shows a schematic of RNA synthesis from the C9orf72 gene in normal and disease states.

FIG. 2 shows a schematic of a precisely targeted humanized C9orf72 expansion allele that reproduces the molecular hallmarks of amyotrophic lateral sclerosis (ALS).

FIG. 3 shows a schematic for generating a C9orf72 allele with a humanized exon 1A promoter.

FIG. 4 shows bar graphs showing expression levels (as determined by the TAQMAN® quantitative reverse transcription-coupled PCR (RT-qPCR) assays shown in the depiction of the C9orf72 locus at the top of each figure) of transcripts from the C9orf72 locus (y-axis) that are exon 1A-exon 2 spliced transcripts, that are exon 1B-exon 2 spliced transcripts, that are exon 5-exon 6 spliced transcripts, that are transcripts containing exon 1A and intron 1 sequence, or that are transcripts containing intron 1 in mouse ES cells that comprise the allele with the humanized C9orf72 promoter region and a humanized C9orf72 locus comprising 3× or 250× repeats of the hexanucleotide sequence. Expression relative to 3× repeat control cells are shown. FIG. 4 shows that the allele with the humanized C9orf72 promoter region reproduces the RNA expression pattern seen in repeat expansion alleles with the mouse promoter, with exon 1A and intron-containing transcripts being expressed at higher levels in 250× repeat mouse ES cells as compared to 3× repeat mouse ES cells, whereas exon 1B transcripts were unchanged.

FIG. 5 shows a schematic of using a catalytically dead version of the Cas9 enzyme (dCas9) and sequence-specific guide RNAs (gRNAs) targeting upstream of the transcription start site for exon 1A to selectively hinder transcriptional initiation and thus expression of exon 1A transcripts but not exon 1B transcripts.

FIG. 6 shows bar graphs showing expression levels (as determined by the TAQMAN® quantitative reverse transcription-coupled PCR (RT-qPCR) assays shown in the depiction of the C9orf72 locus at the top of each figure) of transcripts from the C9orf72 locus (y-axis) that are exon 1A-exon 2 spliced transcripts (top left), that are exon 1B-exon 2 spliced transcripts (top right), that contain exon 1A sequence and intron sequence near exon 1A (bottom left), and that contain intron sequence near exon 1A (bottom right) in mouse ES cells that comprise a humanized C9orf72 locus comprising 300 repeats of the hexanucleotide sequence and a mouse promoter region or in mouse ES cells that comprise a humanized C9orf72 locus comprising 250 repeats of the hexanucleotide sequence and a human promoter region following treatment with dCas9 and sequence-specific guide RNAs (gRNAs) targeting upstream of the transcription start site for exon 1A. Expression levels relative to ES cells treated with a control gRNA are shown.

FIG. 7 shows a schematic of using a catalytically dead version of the Cas9 enzyme (dCas9) and sequence-specific guide RNAs (gRNAs) targeting the C9orf72 hexanucleotide repeat to selectively hinder transcriptional elongation and thus expression of exon 1A transcripts but not exon 1B transcripts.

FIG. 8 shows bar graphs showing expression levels (as determined by the TAQMAN® quantitative reverse transcription-coupled PCR (RT-qPCR) assays shown in the depiction of the C9orf72 locus at the top of each figure) of transcripts from the C9orf72 locus (y-axis) that are exon 1A-exon 2 spliced transcripts (top left), that are exon 1B-exon 2 spliced transcripts (top right), that contain exon 1A sequence and intron sequence near exon 1A (bottom left), and that contain intron sequence near exon 1A (bottom right) in mouse ES cells that comprise a humanized C9orf72 locus comprising 300 repeats of the hexanucleotide sequence and a mouse promoter region or in mouse ES cells that comprise a humanized C9orf72 locus comprising 250 repeats of the hexanucleotide sequence and a human promoter region following treatment with dCas9 and sequence-specific guide RNAs (gRNAs) targeting the hexanucleotide repeat. Expression levels relative to ES cells treated with a control gRNA are shown.

FIG. 9 shows bar graphs (bottom of figure) showing expression levels (as determined by the TAQMAN© quantitative reverse transcription-coupled PCR (RT-qPCR) assays shown in the depiction of the C9orf72 locus at the top of each figure) of transcripts from the C9orf72 locus (y-axis) that are exon 1A-exon 2 spliced transcripts in mouse ES cells that comprise a humanized C9orf72 locus comprising 300 repeats of the hexanucleotide sequence and a mouse promoter region following treatment with dCas9 and sequence-specific guide RNAs (gRNAs) targeting upstream of the transcription start site for exon 1A (top of figure) or targeting the hexanucleotide repeat. Expression levels relative to ES cells treated with a control gRNA are shown.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues, proteins, and nucleic acids includes cells, tissues, proteins, and nucleic acids that are relatively purified with respect to other bacterial, viral, cellular, or other components that may normally be present in situ, up to and including a substantially pure preparation of the cells, tissues, proteins, and nucleic acids. The term “isolated” also includes cells, tissues, proteins, and nucleic acids that have no naturally occurring counterpart, have been chemically synthesized and are thus substantially uncontaminated by other cells, tissues, proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or subject. For example, an endogenous C9orf72 sequence of a human refers to a native C9orf72 sequence that naturally occurs at the C9orf72 locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a polypeptide of interest can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “C9orf72 locus” may refer to the specific location of a C9orf72 gene, C9orf72 DNA sequence, or C9orf72 position on a chromosome of the genome of an organism that has been identified as to where such a sequence resides. A “C9orf72 locus” may comprise a regulatory element of a C9orf72 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a human cell, a human liver cell, or a human liver hepatocyte). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “of” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Sense and antisense repeat expansion C9orf72 RNA detected as cytoplasmic and nuclear foci by fluorescence in situ hybridization (FISH) may sequester RNA binding proteins, leading to cellular toxicity. In addition, dipeptide repeat (DPR) proteins are proposed to be produced from the C9orf72 GGGGCC (G₄C₂) repeat expansion sense and antisense RNA by a non-canonical process that has been termed repeat associated non-AUG (RAN) translation, and there is strong evidence that DPR proteins are cytotoxic. DPR proteins can be translated from all sense and antisense reading frames. Sense DPR proteins include glycine-alanine, glycine-arginine, and glycine-proline DPR proteins. Antisense DPR proteins include proline-arginine, proline-alanine, and glycine-proline. Because G₄C₂ repeat-containing RNAs, either on their own or as templates for dipeptide repeat protein translation, appear to be pathogenic, a general therapeutic strategy is to either inhibit their synthesis or promote their destruction.

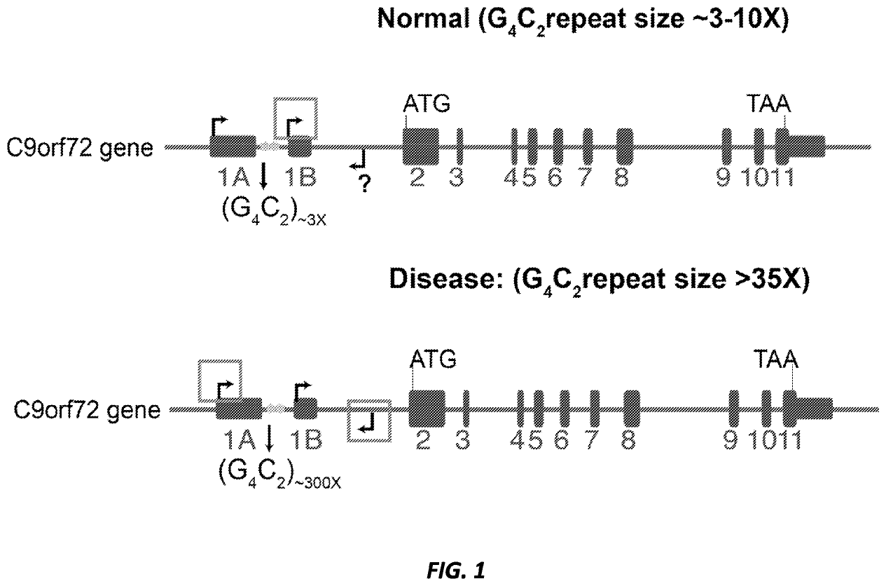

Transcription of the C9orf72 gene initiates at two alternative non-coding exons: exon 1A (upstream) and exon 1B (downstream). The G₄C₂ repeat lies between exons 1A and 1B. Exons 1A and 1B can be spliced to exon 2, the first protein-coding exon, creating mRNAs with alternative 5′-untranslated regions. In healthy people with short G₄C₂ repeat expansions, transcription predominantly initiates at exon 1B; RNAs that include exon 1A are rare, and repeat-containing RNAs are undetectable. People suffering from C9orf72 ALS or FTLD accumulate transcripts in which exon 1A is spliced to exon 2, and both sense and antisense repeat-containing RNAs and the DPR proteins translated from them can be detected by in situ hybridization and immunohistochemistry. See FIG. 1. These pathological findings suggest that the longer disease-associated G₄C₂ repeat expansions promote the use of the upstream exon 1A transcription initiation site, which is the only way that repeat-containing RNAs and their DPR proteins could be produced. It also follows that the production of antisense repeat-containing RNA, which depends on a long repeat expansion, is linked to increased use of the upstream transcription initiation site.

Thus, a possible therapeutic strategy for C9orf72 repeat-expansion disease would be to inhibit or abolish transcription that initiates upstream of the G₄C₂ repeat at exon 1A while retaining transcription that initiates at exon 1B downstream of the repeat, which will retain production of the mRNA for C9orf72 protein synthesis. The CRISPR interference (CRISPRi) technique uses a catalytically dead Cas protein (dCas protein) that lacks endonuclease activity to regulate genes in an RNA-guided manner. CRISPRi can sterically repress transcription by blocking either transcriptional initiation or elongation. This is accomplished by designing guide RNAs complementary to the promoter or coding sequences, respectively. An advantage of CRISPRi over regular Cas9 is that there is no induction of double-strand breaks in DNA and therefore less chance of off-target DNA breaks and DNA damage, which could be very deleterious to the cell and the organism as a whole.

Disclosed herein are guide RNAs and CRISPR/Cas systems targeting a C9orf72 gene, lipid nanoparticles or viral vectors comprising such CRISPR/Cas systems, and cells or animals comprising such CRISPR/Cas systems. Methods of repressing transcription from a C9orf72 exon 1A transcription start site in a C9orf72 gene and/or repressing transcription of sense and/or antisense transcripts that comprise the hexanucleotide repeat expansion sequence using the CRISPR/Cas systems are also provided, as well as use of the CRISPR/Cas systems in prophylactic and therapeutic applications for treatment and/or prevention of a C9orf72 hexanucleotide repeat expansion associated disease and/or for ameliorating at least one symptom associated with such disease. The CRISPR/Cas systems disclosed herein can target promoter elements upstream of or proximate to exon 1A or can target a C9orf72 hexanucleotide repeat expansion sequence between the first non-coding endogenous exon and exon 2 of the C9orf72 gene, selectively or preferentially reducing or abolishing transcripts that initiate at exon 1A or sense or antisense transcripts that comprise the hexanucleotide repeat expansion sequence while retaining transcripts that initiate at exon 1B.

The CRISPR/Cas systems disclosed herein can, for example, reduce or abolish expression of transcripts that initiate at C9orf72 exon 1A. The CRISPR/Cas systems disclosed herein can also, for example, reduce or abolish expression of C9orf72 hexanucleotide-repeat-containing transcripts. The CRISPR/Cas systems disclosed herein can also, for example, reduce or abolish expression of sense C9orf72 hexanucleotide-repeat-containing transcripts. The CRISPR/Cas systems disclosed herein can also, for example, reduce or abolish expression of antisense C9orf72 hexanucleotide-repeat-containing transcripts. The CRISPR/Cas systems disclosed herein can also, for example, reduce or abolish expression of both sense and antisense C9orf72 hexanucleotide-repeat-containing transcripts. The CRISPR/Cas systems disclosed herein can, for example, selectively or preferentially reduce or abolish expression of transcripts that initiate at C9orf72 exon 1A relative to the effect on expression of transcripts that initiate at C9orf72 exon 1B (i.e., reduce expression of transcripts that initiate at C9orf72 exon 1A to a greater extent than reducing expression of transcripts that initiate at C9orf72 exon 1B). The CRISPR/Cas systems disclosed herein can also, for example, selectively or preferentially reduce or abolish expression of C9orf72 hexanucleotide-repeat-containing transcripts relative to the effect on expression of transcripts that initiate at C9orf72 exon 1B (i.e., reduce expression of C9orf72 hexanucleotide-repeat-containing transcripts to a greater extent than reducing expression of transcripts that initiate at C9orf72 exon 1B). The CRISPR/Cas systems disclosed herein can also, for example, selectively or preferentially reduce or abolish expression of sense C9orf72 hexanucleotide-repeat-containing transcripts relative to the effect on expression of transcripts that initiate at C9orf72 exon 1B (e.g., reduce expression of sense C9orf72 hexanucleotide-repeat-containing transcripts to a greater extent than reducing expression of transcripts that initiate at C9orf72 exon 1B). The CRISPR/Cas systems disclosed herein can also, for example, selectively or preferentially reduce or abolish expression of antisense C9orf72 hexanucleotide-repeat-containing transcripts relative to the effect on expression of transcripts that initiate at C9orf72 exon 1B (e.g., reduce expression of antisense C9orf72 hexanucleotide-repeat-containing transcripts to a greater extent than reducing expression of transcripts that initiate at C9orf72 exon 1B). The CRISPR/Cas systems disclosed herein can also, for example, selectively or preferentially reduce or abolish expression of both sense and antisense C9orf72 hexanucleotide-repeat-containing transcripts relative to the effect on expression of transcripts that initiate at C9orf72 exon 1B (e.g., reduce expression of both sense and antisense C9orf72 hexanucleotide-repeat-containing transcripts to a greater extent than reducing expression of transcripts that initiate at C9orf72 exon 1B). In CRISPR/Cas systems disclosed herein, the CRISPR/Cas system reduces or abolishes expression of transcripts that initiate at C9orf72 exon 1A but does not reduce or abolish expression of transcripts that initiate at C9orf72 exon 1B. In some CRISPR/Cas systems disclosed herein, the CRISPR/Cas system reduces or abolishes expression of C9orf72 hexanucleotide-repeat-containing transcripts but does not reduce or abolish expression of transcripts that initiate at C9orf72 exon 1B. In some CRISPR/Cas systems disclosed herein, the CRISPR/Cas system reduces or abolishes expression of sense C9orf72 hexanucleotide-repeat-containing transcripts but does not reduce or abolish expression of transcripts that initiate at C9orf72 exon 1B. In some CRISPR/Cas systems disclosed herein, the CRISPR/Cas system reduces or abolishes expression of antisense C9orf72 hexanucleotide-repeat-containing transcripts but does not reduce or abolish expression of transcripts that initiate at C9orf72 exon 1B. In some CRISPR/Cas systems disclosed herein, the CRISPR/Cas system reduces or abolishes expression of both sense and antisense C9orf72 hexanucleotide-repeat-containing transcripts but does not reduce or abolish expression of transcripts that initiate at C9orf72 exon 1B. In some CRISPR/Cas systems disclosed herein, the CRISPR/Cas system reduces expression of polyGA dipeptide repeat proteins. In some CRISPR/Cas systems disclosed herein, the CRISPR/Cas system reduces expression of polyGP dipeptide repeat proteins. In some CRISPR/Cas systems disclosed herein, the CRISPR/Cas system reduces expression of both polyGA dipeptide repeat proteins and polyGP dipeptide repeat proteins.

### II. CRISPR/Cas Systems Targeting a C9Orf72 Gene

The methods and compositions disclosed herein utilize Clustered Regularly Interspersed Short Palindromic Repeats (CRISPR)/CRISPR-associated (Cas) systems or components of such systems to repress transcription from a C9orf72 exon 1A transcription start site and/or to repress transcription of sense and/or antisense transcripts that comprise the hexanucleotide repeat expansion sequence in a C9orf72 gene within a cell. The C9orf72 can be, for example, a human or mouse C9orf72 or a humanized C9orf72 gene. The cell can be, for example, within a subject, such as a human (e.g., a neuron). The CRISPR/Cas systems disclosed herein use a nuclease-inactive Cas protein (i.e., catalytically dead Cas protein) and can work via a CRISPR interference mechanism to sterically repress transcription. For example, the nuclease-inactive Cas protein can be one that is not fused to a heterologous transcriptional repressor domain, and the guide RNA can be one that is not linked to a heterologous transcriptional repressor domain. The CRISPR interference (CRISPRi) technique uses a catalytically dead Cas protein (dCas protein) that lacks endonuclease activity to regulate genes in an RNA-guided manner. CRISPRi can sterically repress transcription by blocking either transcriptional initiation or elongation. This is accomplished by designing guide RNAs complementary to the promoter or coding sequences, respectively.

### A. C9Orf72, Hexanucleotide Repeat Expansion Sequences, and Associated Diseases

Amyotrophic lateral sclerosis (ALS), also referred to as Lou Gehrig's disease, is the most frequent adult-onset paralytic disorder, characterized by the loss of upper and/or lower motor neurons. ALS occurs in as many as 20,000 individuals across the United States with about 5,000 new cases occurring each year. Frontotemporal dementia (FTD; also referred to as Pick's disease, frontotemporal lobar degeneration, or FTLD) is a group of disorders caused by progressive cell degeneration in the frontal or temporal lobes of the brain. FTD is reported to account for 10%-15% of all dementia cases. A hexanucleotide repeat expansion sequence between exons 1A and 1B, two non-coding exons of the human C9orf72 gene, has been linked to both ALS and FTD. It is estimated that the G₄C₂ hexanucleotide repeat expansion accounts for about 50% of familial and many non-familial ALS cases. It is present in about 25% of familial FTD cases and about 8% of sporadic FTD cases.

Many pathological aspects related to the hexanucleotide repeat expansion sequence in C9orf72 have been reported such as, for example, repeat-length-dependent formation of RNA foci, sequestration of specific RNA-binding proteins, and accumulation and aggregation of dipeptide repeat proteins (e.g., poly(glycine-alanine), poly(glycine-proline), poly(glycine-arginine), poly(alanine-proline), and poly(proline-arginine)) resulting from repeat-associated non-AUG (AUG) translation in their neurons.

Although C9orf72 has been reported to regulate endosomal trafficking, much of the cellular function of C9orf72 remains unknown. Indeed, C9orf72 is a gene that encodes an uncharacterized protein with unknown function.

It is not known how the C9orf72 hexanucleotide repeat expansion causes motor neuron disease and dementia, but two universal postmortem pathological findings in C9orf72 ALS and FTD patients are associated with the repeat expansion: (1) sense and antisense repeat-containing RNA can be visualized as distinct foci in neurons and other cells; and (2) dipeptide repeat proteins—poly(glycine-alanine), poly(glycine-proline), poly(glycine-arginine), poly(alanine-proline), and poly(proline-arginine)—synthesized by repeat-associated non-AUG-dependent translation from the sense and antisense repeat-containing RNAs can be detected in cells. One disease hypothesis proposes that the repeat-containing RNAs, visualized as foci, disrupt cellular RNA metabolism by sequestering RNA binding proteins. Another disease hypothesis posits that the dipeptide repeat proteins exert wide-spread toxic effects on RNA metabolism, proteostasis, and nucleocytoplasmic transport. If C9orf72 repeat-containing RNA transcripts, either on their own or as templates for translation of dipeptide repeat proteins, promote pathogenesis in ALS and FTD, then a general therapeutic strategy would be to inhibit the synthesis of hexanucleotide repeat-containing RNA.

The C9orf72 gene produces transcripts from two transcription initiation sites. The upstream site initiates transcription with alternative non-coding exon 1A, while the downstream site initiates transcription with alternative exon 1B. Both exons 1A and 1B can be spliced to exon 2, which contains the start of the protein-coding sequence. The pathogenic hexanucleotide repeat expansion is located between exons 1A and 1B. Therefore, transcription initiated from exon 1A can produce repeat-containing RNAs, while initiation from exon 1B cannot.

Mouse C9orf72 transcript variants have been reported. See, e.g., Koppers et al. (2015) Ann. Neurol. 78:426-438 and Atkinson et al. (2015) Acta Neuropathologica Communications 3:59, each of which is herein incorporated by reference in its entirety for all purposes. The genomic information for the three reported mouse C9orf72 transcript variants is also available at the Ensembl web site under designations of ENSMUST00000108127 (V1), ENSMUST00000108126 (V2), and ENSMUST00000084724 (V3). Exemplary non-human (e.g., rodent) C9orf72 mRNA and amino acid sequences are set forth in SEQ ID NOS: 22-25. The mRNA and amino acid sequences of mouse C9orf72 can be found at GenBank accession numbers NM_001081343 and NP_001074812, respectively, and are hereby incorporated by reference in their entirety for all purposes. The sequences of NM_001081343.1 and NP_001074812.1 are set forth in SEQ ID NOS: 22 and 23, respectively. The mRNA and amino acid sequences of rat C9orf72 can be found at GenBank accession numbers NM_001007702 and NP_001007703, respectively, and are hereby incorporated by reference in their entirety for all purposes. The sequences of NM_001007702.1 and NP_001007703.1 are set forth in SEQ ID NOS: 24 and 25, respectively.

Human C9orf72 transcript variants are also known. One human C9orf72 transcript variant lacks multiple exons in the central and 3′ coding regions, and its 3′ terminal exon extends beyond a splice site that is used in variant 3 (see below), which results in a novel 3′ untranslated region (UTR) as compared to variant 3. This variant encodes a significantly shorter polypeptide and its C-terminal amino acid is distinct as compared to that which is encoded by two other variants. The mRNA and amino acid sequences of this variant can be found at GenBank accession numbers NM_145005.6 and NP_659442.2, respectively, and are hereby incorporated by reference in their entirety for all purposes. The sequences of NM_145005.6 and NP_659442.2 are set forth in SEQ ID NO: 26 and SEQ ID NO: 27, respectively. A second human C9orf72 transcript variant (2) differs in the 5′ untranslated region (UTR) compared to variant 3. The mRNA and amino acid sequences of this variant can be found at GenBank accession numbers NM_018325.4 and NP_060795.1, respectively, and are hereby incorporated by reference in their entirety for all purposes. The sequences of NM_018325.4 and NP_060795.1 are set forth in SEQ ID NO: 28 and SEQ ID NO: 29, respectively. A third human C9orf72 transcript variant (3) contains the longest sequence among three reported variants and encodes the longer isoform. The mRNA and amino acid sequences of this variant can be found at GenBank accession numbers NM_001256054.2 and NP_001242983.1, respectively, and are hereby incorporated by reference in their entirety for all purposes. The sequences of NM_001256054.2 and NP_001242983.1 are set forth in SEQ ID NO: 30 and SEQ ID NO: 31, respectively. Variants 2 and 3 encode the same protein.

### B. CRISPR/Cas Systems

The methods and compositions disclosed herein utilize Clustered Regularly Interspersed Short Palindromic Repeats (CRISPR)/CRISPR-associated (Cas) systems or components of such systems to repress transcription from a C9orf72 exon 1A transcription start site and/or repress transcription of sense and/or antisense transcripts that comprise the hexanucleotide repeat expansion sequence in a C9orf72 gene within a cell. The CRISPR/Cas systems disclosed herein use a nuclease-inactive Cas protein (i.e., catalytically dead Cas protein) and can work via a CRISPR interference mechanism to sterically repress transcription. For example, the nuclease-inactive Cas protein can be one that is not fused to a heterologous transcriptional repressor domain, and the guide RNA can be one that is not linked to a heterologous transcriptional repressor domain. The CRISPR interference (CRISPRi) technique uses a catalytically dead Cas protein (dCas protein) that lacks endonuclease activity to regulate genes in an RNA-guided manner. CRISPRi can sterically repress transcription by blocking either transcriptional initiation or elongation. This is accomplished by designing guide RNAs complementary to the promoter or coding sequences, respectively.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding of nucleic acids. A CRISPR/Cas system targeting a C9orf72 gene comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus.

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Target Genomic Loci

The guide RNAs and CRISPR/Cas systems described in the compositions and methods disclosed herein target a guide RNA target sequence in a C9orf72 gene. The C9orf72 gene can be, for example, a mammalian C9orf72 gene. In a specific example, the C9orf72 gene comprises a human C9orf72 promoter. In a specific example, the C9orf72 gene is a human C9orf72 gene. In another specific example, the C9orf72 gene is a humanized C9orf72 gene. For example, the C9orf72 gene can be a non-human animal (e.g., non-human mammal, rodent, rat, or mouse) C9orf72 gene in which a human hexanucleotide repeat expansion sequence and flanking human sequence is inserted at an endogenous C9orf72 locus to replace the corresponding endogenous sequence. See, e.g., US 2020-0196581 and WO 2020/131632, each of which is herein incorporated by reference in its entirety for all purposes.

Optionally, the C9orf72 gene comprises a C9orf72 hexanucleotide repeat expansion sequence between the first non-coding endogenous exon and exon 2 of the C9orf72 gene. A C9orf72 hexanucleotide repeat expansion sequence is generally a nucleotide sequence comprising at least two tandem repeats (i.e., contiguous repeats that are adjacent to each other without intervening sequence) of the hexanucleotide sequence G₄C₂. The hexanucleotide repeat expansion sequence can have any number of repeats. Optionally, the hexanucleotide repeat expansion sequence has more than about 30 repeats. In some embodiments, the hexanucleotide repeat expansion sequence has more than about 100 repeats, more than about 200 repeats, more than about 300 repeats, more than about 400 repeats, more than about 500 repeats, more than about 600 repeats, more than about 700 repeats, more than about 800 repeats, more than about 900 repeats, or more than about 1000 repeats.

The guide RNA target sequence(s) can be upstream of or proximate to the C9orf72 exon 1A transcription start site and/or can be within a C9orf72 hexanucleotide repeat expansion sequence between the first non-coding endogenous exon and exon 2 of the C9orf72 gene. Transcription of the C9orf72 gene initiates at two alternative non-coding exons: exon 1A (upstream) and exon 1B (downstream). The G₄C₂ repeat lies between exons 1A and 1B. Exons 1A and 1B can be spliced to exon 2, the first protein-coding exon, creating mRNAs with alternative 5′-untranslated regions. In healthy people with short G₄C₂ repeat expansions, transcription predominantly initiates at exon 1B; RNAs that include exon 1A are rare, and repeat-containing RNAs are undetectable. People suffering from C9orf72 ALS or FTLD accumulate transcripts in which exon 1A is spliced to exon 2, and both sense and antisense repeat-containing RNAs and the DPR proteins translated from them can be detected by in situ hybridization and immunohistochemistry.

In one example, a guide RNA target sequence can be within about 250, within about 225, within about 200, within about 175, within about 150, within about 125, within about 100, within about 75, within about 50, within about 25, within about 20, or within about 10 nucleotides of the C9orf72 exon 1A transcription start site. In another example, a guide RNA target sequence can be within about 100, within about 75, within about 50, within about 25, within about 20, or within about 10 of the C9orf72 exon 1A transcription start site.

In one example, a guide RNA target sequence can be within a C9orf72 hexanucleotide repeat expansion sequence between the first non-coding endogenous exon and exon 2 of the C9orf72 gene.

In some of the compositions and methods disclosed herein, two or more guide RNAs or CRISPR/Cas systems are used to target two or more guide RNA target sequences in a C9orf72 gene. In one example, the two or more guide RNA target sequences are each upstream of or proximate to the C9orf72 exon 1A transcription start site as described above. In another example, the two or more guide RNA target sequences are each within a C9orf72 hexanucleotide repeat expansion sequence between the first non-coding endogenous exon and exon 2 of the C9orf72 gene. In some of the compositions and methods disclosed herein, three or more guide RNAs or CRISPR/Cas systems are used to target three or more guide RNA target sequences in a C9orf72 gene. In one example, the three or more guide RNA target sequences are each upstream of or proximate to the C9orf72 exon 1A transcription start site as described above. In another example, the three or more guide RNA target sequences are each within a C9orf72 hexanucleotide repeat expansion sequence between the first non-coding endogenous exon and exon 2 of the C9orf72 gene. In another example, at least one guide RNA target sequence is upstream of or proximate to the C9orf72 exon 1A transcription start site as described above and at least one guide RNA target sequence is within a C9orf72 hexanucleotide repeat expansion sequence between the first non-coding endogenous exon and exon 2 of the C9orf72 gene.

Some compositions or CRISPR/Cas systems comprise a first guide RNA comprising a first DNA-targeting segment that targets a first guide RNA target sequence upstream of or proximate to the C9orf72 exon 1A transcription start site. For example, the first guide RNA target sequence can be within about 250, within about 225, within about 200, within about 175, within about 150, within about 125, within about 100, within about 75, or within about 50 nucleotides of the C9orf72 exon 1A transcription start site. Alternatively, the first guide RNA target sequence can be within about 100, within about 75, or within about 50 nucleotides of the C9orf72 exon 1A transcription start site or any other position disclosed herein. Some compositions or CRISPR/Cas systems comprise at least two guide RNAs, wherein each different guide RNA targets a different guide RNA target sequence upstream of or proximate to the C9orf72 exon 1A transcription start site. Some compositions or CRISPR/Cas systems comprise at least three guide RNAs, wherein each different guide RNA targets a different guide RNA target sequence upstream of or proximate to the C9orf72 exon 1A transcription start site. For example, each guide RNA target sequence can be within about 250, within about 225, within about 200, within about 175, within about 150, within about 125, within about 100, within about 75, or within about 50 nucleotides of the C9orf72 exon 1A transcription start site. Alternatively, each guide RNA target sequence can be within about 100, within about 75, or within about 50 nucleotides of the C9orf72 exon 1A transcription start site or any other position disclosed herein.

Some compositions or CRISPR/Cas systems comprise a guide RNA comprising a DNA-targeting segment that targets a guide RNA target sequence within a C9orf72 hexanucleotide repeat expansion sequence between the first non-coding endogenous exon and exon 2 of the C9orf72 gene. Some compositions or CRISPR/Cas systems comprise at least two guide RNAs, wherein each different guide RNA targets a different guide RNA target sequence within the C9orf72 hexanucleotide repeat expansion sequence between the first non-coding endogenous exon and exon 2 of the C9orf72 gene. Some compositions or CRISPR/Cas systems comprise at least three guide RNAs, wherein each different guide RNA targets a different guide RNA target sequence within the C9orf72 hexanucleotide repeat expansion sequence between the first non-coding endogenous exon and exon 2 of the C9orf72 gene.

Some compositions or CRISPR/Cas systems comprise: (a) a first guide RNA comprising a first DNA-targeting segment that targets a first guide RNA target sequence upstream of or proximate to the C9orf72 exon 1A transcription start site; and (b) a second guide RNA comprising a second DNA-targeting segment that targets a second guide RNA target sequence within a C9orf72 hexanucleotide repeat expansion sequence between the first non-coding endogenous exon and exon 2 of the C9orf72 gene. In some compositions or CRISPR/Cas systems, part (a) can comprise at least two guide RNAs, wherein each different guide RNA targets a different guide RNA target sequence upstream of or proximate to the C9orf72 exon 1A transcription start site. In some compositions or CRISPR/Cas systems, part (a) can comprise at least three guide RNAs, wherein each different guide RNA targets a different guide RNA target sequence upstream of or proximate to the C9orf72 exon 1A transcription start site. In some compositions or CRISPR/Cas systems, part (b) can comprise at least two guide RNAs, wherein each different guide RNA targets a different guide RNA target sequence within the C9orf72 hexanucleotide repeat expansion sequence between the first non-coding endogenous exon and exon 2 of the C9orf72 gene. In some compositions or CRISPR/Cas systems, part (b) can comprise at least three guide RNAs, wherein each different guide RNA targets a different guide RNA target sequence within the C9orf72 hexanucleotide repeat expansion sequence between the first non-coding endogenous exon and exon 2 of the C9orf72 gene.

### (2) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus. The Cas proteins used in the CRISPR/Cas systems and methods disclosed herein are nuclease-inactive Cas proteins (i.e., catalytically dead Cas proteins).

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 1 (encoded by the DNA sequence set forth in SEQ ID NO: 2). Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPRI locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1; Cas12a) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ) cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be modified Cas proteins (i.e., Cas protein variants) or fragments of wild type or modified Cas proteins.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. See WO 2014/089290, herein incorporated by reference in its entirety for all purposes Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 3) or PKKKRRV (SEQ ID NO: 4). The NLS may be a bipartite sequence, such as the NLS of nucleoplasmin, KRPAATKKAGQAKKKK (SEQ ID NO: 5). In a specific example, a single PKKKRKV (SEQ ID NO: 3) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in a human cell, a human neuron, or a human motor neuron. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In certain embodiments, promotors are accepted by regulatory authorities for use in humans. In certain embodiments, promotors drive expression in a neuron.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV8-mediated delivery). For example, the nuclease agent can be CRISPR/Cas9, and a Cas9 mRNA and a gRNA (e.g., targeting a C9orf72 gene (e.g., a human C9orf72 gene) upstream of or proximate to the exon 1A transcription start site) can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. mRNA encoding Cas proteins can also be capped. Cas mRNAs can further comprise a poly-adenylated (poly-A or poly(A) or poly-adenine) tail. For example, a Cas mRNA can include a modification to one or more nucleosides within the mRNA, the Cas mRNA can be capped, and the Cas mRNA can comprise a poly(A) tail.

### (3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUIUUUAGAGCUAUGCU (SEQ ID NO: 6) or GUUUUAGAGCUAUGCUGUJUJUUUG (SEQ ID NO: 7). Any of the DNA-targeting segments disclosed herein can be joined to the 5′ end of SEQ ID NO: 6 or 7 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 8), AAACAGCAUAGCAAGUUAAAAUAAGGCUAGUCCGUUAUCAACUUGAAAAAGUGG CACCGAGUCGGUGCUUUU (SEQ ID NO: 9), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 10).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 72-91. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 72-91.

As one example, a combination of guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments (i.e., guide sequences) comprising, consisting essentially of, or consisting of the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments comprising, consisting essentially of, or consisting of at least 17, at least 18, at least 19, or at least 20 contiguous nucleotides of the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments that are at least 90% or at least 95% identical to the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to at least 17, at least 18, at least 19, or at least 20 contiguous nucleotides of the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments that are at least 90% or at least 95% identical to at least 17, at least 18, at least 19, or at least 20 contiguous nucleotides of the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments comprising, consisting essentially of, or consisting of sequences that differs by no more than 3, no more than 2, or no more than 1 nucleotide from the sequences (DNA-targeting segments) set forth in one or more or all of SEQ ID NOS: 72-91. Alternatively, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise DNA-targeting segments comprising, consisting essentially of, or consisting of sequences that differs by no more than 3, no more than 2, or no more than 1 nucleotide from at least 17, at least 18, at least 19, or at least 20 contiguous nucleotides of the sequences (DNA-targeting segments) set forth in one or more or all of SEQ ID NOS: 72-91.

As one example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 74. Alternatively, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 74.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 92-111 and 113. Alternatively, a guide RNA targeting a C9orf72 gene upstream of the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 92-111 and 113. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 92-111 and 113.

As one example, a combination of guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments (i.e., guide sequences) comprising, consisting essentially of, or consisting of the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of at least 17, at least 18, at least 19, or at least 20 contiguous nucleotides of the sequences (DNA-targeting segments) set forth in one or more or all of SEQ ID NOS: 92-111 and 113. Alternatively, guide RNAs targeting a C9orf72 gene upstream of the C9orf72 exon 1A transcription start site (e.g., a human C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 90% or at least 95% identical to the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to at least 17, at least 18, at least 19, or at least 20 contiguous nucleotides of the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 90% or at least 95% identical to at least 17, at least 18, at least 19, or at least 20 contiguous nucleotides of the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of sequences that differs by no more than 3, no more than 2, or no more than 1 nucleotide from the sequences (DNA-targeting segments) set forth in one or more or all of SEQ ID NOS: 92-111 and 113. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of a sequence that differs by no more than 3, no more than 2, or no more than 1 nucleotide from at least 17, at least 18, at least 19, or at least 20 contiguous nucleotides of the sequences (DNA-targeting segments) set forth in one or more or all of SEQ ID NOS: 92-111 and 113.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 93-95. Alternatively, a guide RNA targeting a C9orf72 gene upstream of the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 93-95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 93-95.

As one example, a combination of guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments (i.e., guide sequences) comprising, consisting essentially of, or consisting of the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of at least 17, at least 18, at least 19, or at least 20 contiguous nucleotides of the sequences (DNA-targeting segments) set forth in one or more or all of SEQ ID NOS: 93-95. Alternatively, guide RNAs targeting a C9orf72 gene upstream of the C9orf72 exon 1A transcription start site (e.g., a human C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 90% or at least 95% identical to the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 75%, at least 80%, at least 85%, at least 90%, or at least 95% identical to at least 17, at least 18, at least 19, or at least 20 contiguous nucleotides of the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments that are at least 90% or at least 95% identical to at least 17, at least 18, at least 19, or at least 20 contiguous nucleotides of the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of sequences that differs by no more than 3, no more than 2, or no more than 1 nucleotide from the sequences (DNA-targeting segments) set forth in one or more or all of SEQ ID NOS: 93-95. Alternatively, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise DNA-targeting segments comprising, consisting essentially of, or consisting of a sequence that differs by no more than 3, no more than 2, or no more than 1 nucleotide from at least 17, at least 18, at least 19, or at least 20 contiguous nucleotides of the sequences (DNA-targeting segments) set forth in one or more or all of SEQ ID NOS: 93-95.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 93. Alternatively, a guide RNA targeting a C9orf72 gene upstream of the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 93. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 93.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 94. Alternatively, a guide RNA targeting a C9orf72 gene upstream of the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 94. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 94.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment (i.e., guide sequence) comprising, consisting essentially of, or consisting of the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 95. Alternatively, a guide RNA targeting a C9orf72 gene upstream of the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 95. Alternatively, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 95.

As one example, a guide RNA targeting a C9orf72 hexanucleotide repeat expansion sequence can comprise a DNA-targeting segment (i.e., guide sequence) comprising, consisting essentially of, or consisting of the sequence (DNA-targeting segment) set forth in any one of SEQ ID NOS: 118-121. Alternatively, a guide RNA targeting a C9orf72 hexanucleotide repeat expansion sequence can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 118-121. Alternatively, a guide RNA targeting a C9orf72 hexanucleotide repeat expansion sequence can comprise a DNA-targeting segment that is at least 75%, at least 80%, at least 85%, at least 90%, or at least 95% identical to the sequence (DNA-targeting segment) set forth in any one of SEQ ID NOS: 118-121. Alternatively, a guide RNA targeting a C9orf72 hexanucleotide repeat expansion sequence can comprise a DNA-targeting segment that is at least 90% or at least 95% identical to the sequence (DNA-targeting segment) set forth in any one of SEQ ID NOS: 118-121. Alternatively, a guide RNA targeting a C9orf72 hexanucleotide repeat expansion sequence can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 118-121. Alternatively, a guide RNA targeting a C9orf72 hexanucleotide repeat expansion sequence can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 118-121. Alternatively, a guide RNA targeting C9orf72 hexanucleotide repeat expansion sequence can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 118-121. Alternatively, a guide RNA targeting a C9orf72 hexanucleotide repeat expansion sequence can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 118-121.

As one example, a combination of guide RNAs targeting a C9orf72 hexanucleotide repeat expansion sequence can comprise DNA-targeting segments (i.e., guide sequences) comprising, consisting essentially of, or consisting of the sequences (DNA-targeting segments) set forth in one or more or all of SEQ ID NOS: 118-121. Alternatively, guide RNAs targeting a C9orf72 hexanucleotide repeat expansion sequence can comprise DNA-targeting segments comprising, consisting essentially of, or consisting of at least 17, at least 18, at least 19, or at least 20 contiguous nucleotides of the sequences (DNA-targeting segments) set forth in one or more or all of SEQ ID NOS: 118-121. Alternatively, guide RNAs targeting a C9orf72 hexanucleotide repeat expansion sequence can comprise DNA-targeting segments that are at least 75%, at least 80%, at least 85%, at least 90%, or at least 95% identical to the sequences (DNA-targeting segments) set forth in one or more or all of SEQ ID NOS: 118-121. Alternatively, guide RNAs targeting a C9orf72 hexanucleotide repeat expansion sequence can comprise DNA-targeting segments that are at least 90% or at least 95% identical to the sequences (DNA-targeting segments) set forth in one or more or all of SEQ ID NOS: 118-121. Alternatively, guide RNAs targeting a C9orf72 hexanucleotide repeat expansion sequence can comprise DNA-targeting segments that are at least 75%, at least 80%, at least 85%, at least 90%, or at least 95% identical to at least 17, at least 18, at least 19, or at least 20 contiguous nucleotides of the sequences (DNA-targeting segments) set forth in one or more or all of SEQ ID NOS: 118-121. Alternatively, guide RNAs targeting a C9orf72 hexanucleotide repeat expansion sequence can comprise DNA-targeting segments that are at least 90% or at least 95% identical to at least 17, at least 18, at least 19, or at least 20 contiguous nucleotides of the sequences (DNA-targeting segments) set forth in one or more or all of SEQ ID NOS: 118-121. Alternatively, guide RNAs targeting C9orf72 hexanucleotide repeat expansion sequence can comprise DNA-targeting segments comprising, consisting essentially of, or consisting of a sequence that differs by no more than 3, no more than 2, or no more than 1 nucleotide from the sequences (DNA-targeting segments) set forth in any one of SEQ ID NOS: 118-121. Alternatively, guide RNAs targeting a C9orf72 hexanucleotide repeat expansion sequence can comprise DNA-targeting segments comprising, consisting essentially of, or consisting of sequences that differ by no more than 3, no more than 2, or no more than 1 nucleotide from at least 17, at least 18, at least 19, or at least 20 contiguous nucleotides of the sequences (DNA-targeting segments) set forth in one or more or all of SEQ ID NOS: 118-121.

As one example, a guide RNA targeting a C9orf72 hexanucleotide repeat expansion sequence can comprise a DNA-targeting segment (i.e., guide sequence) comprising, consisting essentially of, or consisting of the sequence (DNA-targeting segment) set forth in SEQ ID NO: 118. Alternatively, a guide RNA targeting a C9orf72 hexanucleotide repeat expansion sequence can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 118. Alternatively, a guide RNA targeting a C9orf72 hexanucleotide repeat expansion sequence can comprise a DNA-targeting segment that is at least 75%, at least 80%, at least 85%, at least 90%, or at least 95% identical to the sequence (DNA-targeting segment) set forth in SEQ ID NO: 118. Alternatively, a guide RNA targeting a C9orf72 hexanucleotide repeat expansion sequence can comprise a DNA-targeting segment that is at least 90% or at least 95% identical to the sequence (DNA-targeting segment) set forth in SEQ ID NO: 118. Alternatively, a guide RNA targeting a C9orf72 hexanucleotide repeat expansion sequence can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 118. Alternatively, a guide RNA targeting a C9orf72 hexanucleotide repeat expansion sequence can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 118. Alternatively, a guide RNA targeting C9orf72 hexanucleotide repeat expansion sequence can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 118. Alternatively, a guide RNA targeting a C9orf72 hexanucleotide repeat expansion sequence can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 118.

As one example, a guide RNA targeting a C9orf72 hexanucleotide repeat expansion sequence can comprise a DNA-targeting segment (i.e., guide sequence) comprising, consisting essentially of, or consisting of the sequence (DNA-targeting segment) set forth in SEQ ID NO: 119. Alternatively, a guide RNA targeting a C9orf72 hexanucleotide repeat expansion sequence can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 119. Alternatively, a guide RNA targeting a C9orf72 hexanucleotide repeat expansion sequence can comprise a DNA-targeting segment that is at least 75%, at least 80%, at least 85%, at least 90%, or at least 95% identical to the sequence (DNA-targeting segment) set forth in SEQ ID NO: 119. Alternatively, a guide RNA targeting a C9orf72 hexanucleotide repeat expansion sequence can comprise a DNA-targeting segment that is at least 90% or at least 95% identical to the sequence (DNA-targeting segment) set forth in SEQ ID NO: 119. Alternatively, a guide RNA targeting a C9orf72 hexanucleotide repeat expansion sequence can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 119. Alternatively, a guide RNA targeting a C9orf72 hexanucleotide repeat expansion sequence can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 119. Alternatively, a guide RNA targeting C9orf72 hexanucleotide repeat expansion sequence can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 119. Alternatively, a guide RNA targeting a C9orf72 hexanucleotide repeat expansion sequence can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 119.

As one example, a guide RNA targeting a C9orf72 hexanucleotide repeat expansion sequence can comprise a DNA-targeting segment (i.e., guide sequence) comprising, consisting essentially of, or consisting of the sequence (DNA-targeting segment) set forth in SEQ ID NO: 120. Alternatively, a guide RNA targeting a C9orf72 hexanucleotide repeat expansion sequence can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 120. Alternatively, a guide RNA targeting a C9orf72 hexanucleotide repeat expansion sequence can comprise a DNA-targeting segment that is at least 75%, at least 80%, at least 85%, at least 90%, or at least 95% identical to the sequence (DNA-targeting segment) set forth in SEQ ID NO: 120. Alternatively, a guide RNA targeting a C9orf72 hexanucleotide repeat expansion sequence can comprise a DNA-targeting segment that is at least 90% or at least 95% identical to the sequence (DNA-targeting segment) set forth in SEQ ID NO: 120. Alternatively, a guide RNA targeting a C9orf72 hexanucleotide repeat expansion sequence can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 120. Alternatively, a guide RNA targeting a C9orf72 hexanucleotide repeat expansion sequence can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 120. Alternatively, a guide RNA targeting C9orf72 hexanucleotide repeat expansion sequence can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 120. Alternatively, a guide RNA targeting a C9orf72 hexanucleotide repeat expansion sequence can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 120.

As one example, a guide RNA targeting a C9orf72 hexanucleotide repeat expansion sequence can comprise a DNA-targeting segment (i.e., guide sequence) comprising, consisting essentially of, or consisting of the sequence (DNA-targeting segment) set forth in SEQ ID NO: 121. Alternatively, a guide RNA targeting a C9orf72 hexanucleotide repeat expansion sequence can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 121. Alternatively, a guide RNA targeting a C9orf72 hexanucleotide repeat expansion sequence can comprise a DNA-targeting segment that is at least 75%, at least 80%, at least 85%, at least 90%, or at least 95% identical to the sequence (DNA-targeting segment) set forth in SEQ ID NO: 121. Alternatively, a guide RNA targeting a C9orf72 hexanucleotide repeat expansion sequence can comprise a DNA-targeting segment that is at least 90% or at least 95% identical to the sequence (DNA-targeting segment) set forth in SEQ ID NO: 121. Alternatively, a guide RNA targeting a C9orf72 hexanucleotide repeat expansion sequence can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 121. Alternatively, a guide RNA targeting a C9orf72 hexanucleotide repeat expansion sequence can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 121. Alternatively, a guide RNA targeting C9orf72 hexanucleotide repeat expansion sequence can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 121. Alternatively, a guide RNA targeting a C9orf72 hexanucleotide repeat expansion sequence can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 121.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 11); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 12); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 13); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 14); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 15); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 16); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 17); or GUUUUAGAGCUAGAAAUAGCAAGUUAAAAUAAGGCUAGUCCGUUAUCAACUUGG CACCGAGUCGGUGC (version 8; SEQ ID NO: 18). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification. Some gRNAs comprise a 5′ end modification and a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in a human cell, a human neuron, or a human motor neuron. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

### (4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, where B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 19) or N₂₀NGG (SEQ ID NO: 20). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 21) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 19-21, including the 5′ G or GG and the 3′ GG or NGG. Yet other guide RNA target sequences plus PAMs can have between 14 and 20 nucleotides in length of SEQ ID NOS: 19-21.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target the guide RNA target sequence set forth in any one of SEQ ID NOS: 32-51. As another example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target at least 17, at least 18, at least 19, or at least 20 contiguous nucleotides of the guide RNA target sequence set forth in any one of SEQ ID NOS: 32-51.

As one example, a combination of guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target the guide RNA target sequences set forth in one or more or all of SEQ ID NOS: 32-51. As another example, guide RNAs targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target at least 17, at least 18, at least 19, or at least 20 contiguous nucleotides of the guide RNA target sequences set forth in one or more or all of SEQ ID NOS: 32-51.

As one example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target the guide RNA target sequence set forth in SEQ ID NO: 34. As another example, a guide RNA targeting a C9orf72 gene upstream of or proximate to the C9orf72 exon 1A transcription start site (e.g., a mouse C9orf72 guide RNA target sequence upstream of or proximate to the C9orf72 exon 1A transcription start site) can target at least 17, at least 18, at least 19, or at least 20 contiguous nucleotides of the guide RNA target sequence set forth in SEQ ID NO: 34.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequence set forth in any one of SEQ ID NOS: 52-71 and 112. As another example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequence set forth in any one of SEQ ID NOS: 52-71 and 112.

As one example, a combination of guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequences set forth in one or more or all of SEQ ID NOS: 52-71 and 112. As another example, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequences set forth in one or more or all of SEQ ID NOS: 52-71 and 112.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequence set forth in any one of SEQ ID NOS: 53-55. As another example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequence set forth in any one of SEQ ID NOS: 53-55.

As one example, a combination of guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequences set forth in one or more or all of SEQ ID NOS: 53-55 and 112. As another example, guide RNAs targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequences set forth in one or more or all of SEQ ID NOS: 53-55.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequence set forth in SEQ ID NO: 53. As another example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequence set forth in SEQ ID NO: 53.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequence set forth in SEQ ID NO: 54. As another example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequence set forth in SEQ ID NO: 54.

As one example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target the guide RNA target sequence set forth in SEQ ID NO: 55. As another example, a guide RNA targeting a C9orf72 gene upstream of or proximate to the C9orf72 exon 1A transcription start site (e.g., a human C9orf72 guide RNA target sequence upstream of or proximate to the C9orf72 exon 1A transcription start site) can target at least 17, at least 18, at least 19, or at least 20 contiguous nucleotides of the guide RNA target sequence set forth in SEQ ID NO: 55.

As one example, a guide RNA targeting a C9orf72 hexanucleotide repeat expansion sequence can target the guide RNA target sequence set forth in any one of SEQ ID NOS: 114-117. As another example, a guide RNA targeting a C9orf72 hexanucleotide repeat expansion sequence can target at least 17, at least 18, at least 19, or at least 20 contiguous nucleotides of the guide RNA target sequence set forth in any one of SEQ ID NOS: 114-117.

As one example, a combination of guide RNAs targeting a C9orf72 hexanucleotide repeat expansion sequence can target the guide RNA target sequences set forth in one or more or all of SEQ ID NOS: 114-117. As another example, guide RNAs targeting a C9orf72 hexanucleotide repeat expansion sequence can target at least 17, at least 18, at least 19, or at least 20 contiguous nucleotides of the guide RNA target sequences set forth in one or more or all of SEQ ID NOS: 114-117.

As one example, a guide RNA targeting a C9orf72 hexanucleotide repeat expansion sequence can target the guide RNA target sequence set forth in SEQ ID NO: 114. As another example, a guide RNA targeting a C9orf72 hexanucleotide repeat expansion sequence can target at least 17, at least 18, at least 19, or at least 20 contiguous nucleotides of the guide RNA target sequence set forth in SEQ ID NO: 114.

As one example, a guide RNA targeting a C9orf72 hexanucleotide repeat expansion sequence can target the guide RNA target sequence set forth in SEQ ID NO: 115. As another example, a guide RNA targeting a C9orf72 hexanucleotide repeat expansion sequence can target at least 17, at least 18, at least 19, or at least 20 contiguous nucleotides of the guide RNA target sequence set forth in SEQ ID NO: 115.

As one example, a guide RNA targeting a C9orf72 hexanucleotide repeat expansion sequence can target the guide RNA target sequence set forth in SEQ ID NO: 116. As another example, a guide RNA targeting a C9orf72 hexanucleotide repeat expansion sequence can target at least 17, at least 18, at least 19, or at least 20 contiguous nucleotides of the guide RNA target sequence set forth in SEQ ID NO: 116.

As one example, a guide RNA targeting a C9orf72 hexanucleotide repeat expansion sequence can target the guide RNA target sequence set forth in SEQ ID NO: 117. As another example, a guide RNA targeting a C9orf72 hexanucleotide repeat expansion sequence can target at least 17, at least 18, at least 19, or at least 20 contiguous nucleotides of the guide RNA target sequence set forth in SEQ ID NO: 117.

### (5) Lipid Nanoparticles Comprising CRISPR/Cas Systems or Components

Lipid nanoparticles comprising the CRISPR/Cas systems or components thereof are also provided. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. See, e.g., WO 2016/010840 A1 and WO 2017/173054 A1, each of which is herein incorporated by reference in its entirety for all purposes. An exemplary lipid nanoparticle can comprise a cationic lipid and one or more other components.

In some LNPs, the cargo can comprise Cas mRNA (e.g., Cas9 mRNA) and gRNA. The Cas mRNA and gRNAs can be in different ratios.

Examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

### (6) Vectors Comprising CRISPR/Cas Systems or Components

The CRISPR/Cas systems or components thereof can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes, a gene expression cassette can be placed between ITR sequences. Typically, rAAV genome cassettes comprise of a promoter to drive expression of a transgene, followed by a polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas protein, such as Cas9, and DNA encoding one or more guide RNAs (e.g., DNA encoding a guide RNA, or DNA encoding two or more guide RNAs).

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery. For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### III. Methods for Repressing Transcription from a C9Orf72 Exon 1A Transcription Start Site in a Cell or a Subject and Methods of Preventing, Treating, or Ameliorating at Least One Symptom or Indication of a C9Orf72 Hexanucleotide Repeat Expansion Associated Disease

Also disclosed herein are methods of repressing transcription from a C9orf72 exon 1A transcription start site and/or repressing transcription of sense and/or antisense transcripts that comprise the hexanucleotide repeat expansion sequence using the CRISPR/Cas systems described herein, as well as use of the CRISPR/Cas systems in prophylactic and therapeutic applications for treatment and/or prevention of C9orf72 hexanucleotide repeat expansion associated disease and/or for ameliorating at least one symptom associated with such disease. Such methods can, for example, reduce or abolish expression of transcripts that initiate at C9orf72 exon 1A. Such methods can also, for example, reduce or abolish expression of C9orf72 hexanucleotide-repeat-containing transcripts. Such methods can also, for example, reduce or abolish expression of sense C9orf72 hexanucleotide-repeat-containing transcripts. Such methods can also, for example, reduce or abolish expression of antisense C9orf72 hexanucleotide-repeat-containing transcripts. Such methods can also, for example, reduce or abolish expression of both sense and antisense C9orf72 hexanucleotide-repeat-containing transcripts. Such methods can, for example, selectively or preferentially reduce or abolish expression of transcripts that initiate at C9orf72 exon 1A relative to the effect on expression of transcripts that initiate at C9orf72 exon 1B (i.e., reduce expression of transcripts that initiate at C9orf72 exon 1A to a greater extent than reducing expression of transcripts that initiate at C9orf72 exon 1B). Such methods can also, for example, selectively or preferentially reduce or abolish expression of C9orf72 hexanucleotide-repeat-containing transcripts relative to the effect on expression of transcripts that initiate at C9orf72 exon 1B (i.e., reduce expression of C9orf72 hexanucleotide-repeat-containing transcripts to a greater extent than reducing expression of transcripts that initiate at C9orf72 exon 1B). Such methods can also, for example, selectively or preferentially reduce or abolish expression of sense C9orf72 hexanucleotide-repeat-containing transcripts relative to the effect on expression of transcripts that initiate at C9orf72 exon 1B (e.g., reduce expression of sense C9orf72 hexanucleotide-repeat-containing transcripts to a greater extent than reducing expression of transcripts that initiate at C9orf72 exon 1B). Such methods can also, for example, selectively or preferentially reduce or abolish expression of antisense C9orf72 hexanucleotide-repeat-containing transcripts relative to the effect on expression of transcripts that initiate at C9orf72 exon 1B (e.g., reduce expression of antisense C9orf72 hexanucleotide-repeat-containing transcripts to a greater extent than reducing expression of transcripts that initiate at C9orf72 exon 1B). Such methods can also, for example, selectively or preferentially reduce or abolish expression of both sense and antisense C9orf72 hexanucleotide-repeat-containing transcripts relative to the effect on expression of transcripts that initiate at C9orf72 exon 1B (e.g., reduce expression of both sense and antisense C9orf72 hexanucleotide-repeat-containing transcripts to a greater extent than reducing expression of transcripts that initiate at C9orf72 exon 1B). In some methods, the CRISPR/Cas system reduces or abolishes expression of transcripts that initiate at C9orf72 exon 1A but does not reduce or abolish expression of transcripts that initiate at C9orf72 exon 1B. In some methods, the CRISPR/Cas system reduces or abolishes expression of C9orf72 hexanucleotide-repeat-containing transcripts but does not reduce or abolish expression of transcripts that initiate at C9orf72 exon 1B. In some methods, the CRISPR/Cas system reduces or abolishes expression of sense C9orf72 hexanucleotide-repeat-containing transcripts but does not reduce or abolish expression of transcripts that initiate at C9orf72 exon 1B. In some methods, the CRISPR/Cas system reduces or abolishes expression of antisense C9orf72 hexanucleotide-repeat-containing transcripts but does not reduce or abolish expression of transcripts that initiate at C9orf72 exon 1B. In some methods, the CRISPR/Cas system reduces or abolishes expression of both sense and antisense C9orf72 hexanucleotide-repeat-containing transcripts but does not reduce or abolish expression of transcripts that initiate at C9orf72 exon 1B. In some methods disclosed herein, the CRISPR/Cas system reduces expression of polyGA dipeptide repeat proteins. In some methods disclosed herein, the CRISPR/Cas system reduces expression of polyGP dipeptide repeat proteins. In some methods disclosed herein, the CRISPR/Cas system reduces expression of both polyGA dipeptide repeat proteins and polyGP dipeptide repeat proteins.

A. Methods of Repressing Transcription from a C9Orf72 Exon 1A Transcription Start Site in a Cell or a Subject

Various methods are provided for repressing transcription from a C9orf72 exon 1A transcription start site and/or repressing transcription of sense and/or antisense transcripts that comprise the hexanucleotide repeat expansion sequence using the CRISPR/Cas systems (e.g., Cas protein or a nucleic acid encoding and one or more C9orf72-targeting gRNAs or DNAs encoding) described elsewhere herein.

The C9orf72 gene can be in an animal or cell, and the methods can occur in vitro, ex vivo, or in vivo. Animals include mammals, fishes, and birds. A mammal can be, for example, a non-human mammal, a human, a rodent, a rat, a mouse, or a hamster. In one example, the animal is a human. Other non-human mammals include, for example, non-human primates (e.g., cynomolgus), monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The animals in the methods disclosed herein can be humans or they can be non-human animals. In a specific example, the C9orf72 gene is in a human or a human cell. The term “non-human” excludes humans. Particular examples of non-human animals include rodents, such as mice and rats, or non-human primates, such as cynomolgus.

Cells used in the methods can be from any type of animal, and they can be any type of undifferentiated or differentiated state. The cells can be in vitro, ex vivo, or in vivo. For example, a cell can be a non-human totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. In some embodiments, a human cell is a not a totipotent cell. In some embodiments, a human cell is not a pluripotent cell.

The cells provided herein can also be germ cells (e.g., sperm or oocytes) or non-human germ cells. The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, kidney cells, hematopoietic cells, endothelial cells, epithelial cells, fibroblasts, mesenchymal cells, keratinocytes, blood cells, melanocytes, monocytes, mononuclear cells, monocytic precursors, B cells, erythroid-megakaryocytic cells, eosinophils, macrophages, T cells, islet beta cells, exocrine cells, pancreatic progenitors, endocrine progenitors, adipocytes, preadipocytes, neurons, glial cells, neural stem cells, neurons, hepatoblasts, hepatocytes, cardiomyocytes, skeletal myoblasts, smooth muscle cells, ductal cells, acinar cells, alpha cells, beta cells, delta cells, PP cells, cholangiocytes, white or brown adipocytes, or ocular cells (e.g., trabecular meshwork cells, retinal pigment epithelial cells, retinal microvascular endothelial cells, retinal pericyte cells, conjunctival epithelial cells, conjunctival fibroblasts, iris pigment epithelial cells, keratocytes, lens epithelial cells, non-pigment ciliary epithelial cells, ocular choroid fibroblasts, photoreceptor cells, ganglion cells, bipolar cells, horizontal cells, or amacrine cells). For example, the cells can be neurons, such as motor neurons. The cells provided herein can be normal, healthy cells, or can be diseased or mutant-bearing cells such as cells comprising a hexanucleotide repeat expansion at the C9orf72 locus.

Non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 129S9/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. In some cases, suitable rats can be from an inbred rat strain. See, e.g., US 2014/0235933, herein incorporated by reference in its entirety for all purposes.

The CRISPR/Cas reagents can be introduced into a cell or an animal in any form and by any means as described elsewhere herein, and all or some can be introduced simultaneously or sequentially in any combination as described elsewhere herein.

In some methods, the method comprises contacting the C9orf72 gene with a first CRISPR/Cas complex comprising a nuclease-inactive Cas protein and a first guide RNA comprising a first DNA-targeting segment that targets a first guide RNA target sequence upstream of or proximate to the C9orf72 exon 1A transcription start site, wherein the first CRISPR/Cas complex binds to the first guide RNA target sequence. For example, the first guide RNA target sequence can be within about 250, within about 225, within about 200, within about 175, within about 150, within about 125, within about 100, within about 75, or within about 50 nucleotides of the C9orf72 exon 1A transcription start site. Alternatively, the first guide RNA target sequence can be within about 100, within about 75, or within about 50 nucleotides of the C9orf72 exon 1A transcription start site or any other position disclosed herein. In some methods, the method can comprise contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site. In another example, the method can comprise contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site. For example, each guide RNA target sequence can be within about 250, within about 225, within about 200, within about 175, within about 150, within about 125, within about 100, within about 75, or within about 50 nucleotides of the C9orf72 exon 1A transcription start site. Alternatively, each guide RNA target sequence can be within about 100, within about 75, or within about 50 nucleotides of the C9orf72 exon 1A transcription start site or any other position disclosed herein.

In some methods, the method comprises contacting the C9orf72 gene with a second CRISPR/Cas complex comprising the nuclease-inactive Cas protein and a second guide RNA comprising a second DNA-targeting segment that targets a second guide RNA target sequence within a C9orf72 hexanucleotide repeat expansion sequence between the first non-coding endogenous exon and exon 2 of the C9orf72 gene, wherein the second CRISPR/Cas complex binds to the first guide RNA target sequence. In some methods, the method can comprise contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene. In some methods, the method can comprise contacting the C9orf72 gene with at least three or at least four CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene.

In some methods, the method can comprise: (a) contacting the C9orf72 gene with a first CRISPR/Cas complex comprising a nuclease-inactive Cas protein and a first guide RNA comprising a first DNA-targeting segment that targets a first guide RNA target sequence upstream of or proximate to the C9orf72 exon 1A transcription start site, wherein the first CRISPR/Cas complex binds to the first guide RNA target sequence; and (b) contacting the C9orf72 gene with a second CRISPR/Cas complex comprising the nuclease-inactive Cas protein and a second guide RNA comprising a second DNA-targeting segment that targets a second guide RNA target sequence within a C9orf72 hexanucleotide repeat expansion sequence between the first non-coding endogenous exon and exon 2 of the C9orf72 gene, wherein the second CRISPR/Cas complex binds to the first guide RNA target sequence. In some methods, part (a) can comprise contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site. In some methods, part (a) can comprise contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site. In some methods, part (b) can comprise contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene. In some methods, part (b) can comprise contacting the C9orf72 gene with at least three or at least four CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene.

Examples and variations of Cas proteins and guide RNAs that can be used in the methods are described elsewhere herein.

A guide RNA can be introduced into an animal or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the animal. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into an animal or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or an animal, the Cas protein can be transiently, conditionally, or constitutively expressed in the cell or in a cell in the animal.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle).

Guide RNAs can be modified as disclosed elsewhere herein. Likewise, Cas mRNAs can be modified as disclosed elsewhere herein.

The guide RNAs and CRISPR/Cas systems described in the compositions and methods disclosed herein target a guide RNA target sequence in a C9orf72 gene. The C9orf72 gene can be, for example, a mammalian C9orf72 gene. In a specific example, the C9orf72 gene comprises a human C9orf72 promoter. In a specific example, the C9orf72 gene is a human C9orf72 gene. In another specific example, the C9orf72 gene is a humanized C9orf72 gene. For example, the C9orf72 gene can be a non-human animal (e.g., non-human mammal, rodent, rat, or mouse) C9orf72 gene in which a human hexanucleotide repeat expansion sequence and flanking human sequence is inserted at an endogenous C9orf72 locus to replace the corresponding endogenous sequence. See, e.g., US 2020-0196581 and WO 2020/131632, each of which is herein incorporated by reference in its entirety for all purposes.

Optionally, the C9orf72 gene comprises a C9orf72 hexanucleotide repeat expansion sequence between the first non-coding endogenous exon and exon 2 of the C9orf72 gene. A C9orf72 hexanucleotide repeat expansion sequence is generally a nucleotide sequence comprising at least two tandem repeats (i.e., contiguous repeats that are adjacent to each other without intervening sequence) of the hexanucleotide sequence G₄C₂. The hexanucleotide repeat expansion sequence can have any number of repeats. Optionally, the hexanucleotide repeat expansion sequence has more than about 30 repeats. In some embodiments, the hexanucleotide repeat expansion sequence has more than about 100 repeats, more than about 200 repeats, more than about 300 repeats, more than about 400 repeats, more than about 500 repeats, more than about 600 repeats, more than about 700 repeats, more than about 800 repeats, more than about 900 repeats, or more than about 1000 repeats.

The guide RNA target sequence(s) can be upstream of or proximate to the C9orf72 exon 1A transcription start site and/or can be within a C9orf72 hexanucleotide repeat expansion sequence between the first non-coding endogenous exon and exon 2 of the C9orf72 gene. Transcription of the C9orf72 gene initiates at two alternative non-coding exons: exon 1A (upstream) and exon 1B (downstream). The G₄C₂ repeat lies between exons 1A and 1B. Exons 1A and 1B can be spliced to exon 2, the first protein-coding exon, creating mRNAs with alternative 5′-untranslated regions. In healthy people with short G₄C₂ repeat expansions, transcription predominantly initiates at exon 1B; RNAs that include exon 1A are rare, and repeat-containing RNAs are undetectable. People suffering from C9orf72 ALS or FTLD accumulate transcripts in which exon 1A is spliced to exon 2, and both sense and antisense repeat-containing RNAs and the DPR proteins translated from them can be detected by in situ hybridization and immunohistochemistry.

In one example, a guide RNA target sequence can be within about 250, within about 225, within about 200, within about 175, within about 150, within about 125, within about 100, within about 75, within about 50, within about 25, within about 20, or within about 10 nucleotides of the C9orf72 exon 1A transcription start site. In another example, a guide RNA target sequence can be within about 100, within about 75, within about 50, within about 25, within about 20, or within about 10 of the C9orf72 exon 1A transcription start site.

In one example, a guide RNA target sequence can be within a C9orf72 hexanucleotide repeat expansion sequence between the first non-coding endogenous exon and exon 2 of the C9orf72 gene.

In some of the compositions and methods disclosed herein, two or more guide RNAs or CRISPR/Cas systems are used to target two or more guide RNA target sequences in a C9orf72 gene. In one example, the two or more guide RNA target sequences are each upstream of or proximate to the C9orf72 exon 1A transcription start site as described above. In another example, the two or more guide RNA target sequences are each within a C9orf72 hexanucleotide repeat expansion sequence between the first non-coding endogenous exon and exon 2 of the C9orf72 gene. In some of the compositions and methods disclosed herein, three or more guide RNAs or CRISPR/Cas systems are used to target three or more guide RNA target sequences in a C9orf72 gene. In one example, the three or more guide RNA target sequences are each upstream of or proximate to the C9orf72 exon 1A transcription start site as described above. In another example, the three or more guide RNA target sequences are each within a C9orf72 hexanucleotide repeat expansion sequence between the first non-coding endogenous exon and exon 2 of the C9orf72 gene. In another example, at least one guide RNA target sequence is upstream of or proximate to the C9orf72 exon 1A transcription start site as described above and at least one guide RNA target sequence is within a C9orf72 hexanucleotide repeat expansion sequence between the first non-coding endogenous exon and exon 2 of the C9orf72 gene.

Guide RNA target sequences can also be selected to minimize or avoid off-target effects (e.g., by avoiding two or fewer mismatches to off-target genomic sequences).

Methods for measuring expression of transcripts that initiate at C9orf72 exon 1A, expression of transcripts that initiate at C9orf72 exon 1B, and expression of sense and antisense C9orf72 hexanucleotide-repeat-containing transcripts are known and are described elsewhere herein. Assessment of expression (or measuring RNA foci and dipeptide repeats as described elsewhere herein) can be in any cell type (e.g., neurons, such as motor neurons).

In some methods, the resulting percent expression from the C9orf72 exon 1A transcription start site (i.e., expression of transcripts comprising exon 1A) in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression from the C9orf72 exon 1A transcription start site in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of sense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of sense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of antisense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of antisense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of both sense and antisense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of both sense and antisense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGA dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGP dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGA and polyGP dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

Such methods can, for example, reduce or abolish expression of transcripts that initiate at C9orf72 exon 1A. Such methods can also, for example, reduce or abolish expression of C9orf72 hexanucleotide-repeat-containing transcripts. Such methods can also, for example, reduce or abolish expression of sense C9orf72 hexanucleotide-repeat-containing transcripts. Such methods can also, for example, reduce or abolish expression of antisense C9orf72 hexanucleotide-repeat-containing transcripts. Such methods can also, for example, reduce or abolish expression of both sense and antisense C9orf72 hexanucleotide-repeat-containing transcripts. Such methods can, for example, selectively or preferentially reduce or abolish expression of transcripts that initiate at C9orf72 exon 1A relative to the effect on expression of transcripts that initiate at C9orf72 exon 1B (i.e., reduce expression of transcripts that initiate at C9orf72 exon 1A to a greater extent than reducing expression of transcripts that initiate at C9orf72 exon 1B). Such methods can also, for example, selectively or preferentially reduce or abolish expression of C9orf72 hexanucleotide-repeat-containing transcripts relative to the effect on expression of transcripts that initiate at C9orf72 exon 1B (i.e., reduce expression of C9orf72 hexanucleotide-repeat-containing transcripts to a greater extent than reducing expression of transcripts that initiate at C9orf72 exon 1B). Such methods can also, for example, selectively or preferentially reduce or abolish expression of sense C9orf72 hexanucleotide-repeat-containing transcripts relative to the effect on expression of transcripts that initiate at C9orf72 exon 1B (e.g., reduce expression of sense C9orf72 hexanucleotide-repeat-containing transcripts to a greater extent than reducing expression of transcripts that initiate at C9orf72 exon 1B). Such methods can also, for example, selectively or preferentially reduce or abolish expression of antisense C9orf72 hexanucleotide-repeat-containing transcripts relative to the effect on expression of transcripts that initiate at C9orf72 exon 1B (e.g., reduce expression of antisense C9orf72 hexanucleotide-repeat-containing transcripts to a greater extent than reducing expression of transcripts that initiate at C9orf72 exon 1B). Such methods can also, for example, selectively or preferentially reduce or abolish expression of both sense and antisense C9orf72 hexanucleotide-repeat-containing transcripts relative to the effect on expression of transcripts that initiate at C9orf72 exon 1B (e.g., reduce expression of both sense and antisense C9orf72 hexanucleotide-repeat-containing transcripts to a greater extent than reducing expression of transcripts that initiate at C9orf72 exon 1B). In some methods, the CRISPR/Cas system reduces or abolishes expression of transcripts that initiate at C9orf72 exon 1A but does not reduce or abolish expression of transcripts that initiate at C9orf72 exon 1B. In some methods, the CRISPR/Cas system reduces or abolishes expression of C9orf72 hexanucleotide-repeat-containing transcripts but does not reduce or abolish expression of transcripts that initiate at C9orf72 exon 1B. In some methods, the CRISPR/Cas system reduces or abolishes expression of sense C9orf72 hexanucleotide-repeat-containing transcripts but does not reduce or abolish expression of transcripts that initiate at C9orf72 exon 1B. In some methods, the CRISPR/Cas system reduces or abolishes expression of antisense C9orf72 hexanucleotide-repeat-containing transcripts but does not reduce or abolish expression of transcripts that initiate at C9orf72 exon 1B. In some methods, the CRISPR/Cas system reduces or abolishes expression of both sense and antisense C9orf72 hexanucleotide-repeat-containing transcripts but does not reduce or abolish expression of transcripts that initiate at C9orf72 exon 1B.

Some methods result in a decrease in sense and/or antisense repeat-containing RNA foci in treated cells (e.g., neurons, such as motor neurons) as compared to control untreated cells or as compared to pre-administration (in vitro, ex vivo, or in vivo). Some methods result in a decrease in dipeptide repeat proteins (e.g., poly(glycine-alanine), poly(glycine-proline), poly(glycine-arginine), poly(alanine-proline), and/or poly(proline-arginine)) synthesized by repeat-associated non-AUG-dependent translation from the sense and antisense repeat-containing RNAs in treated cells (e.g., neurons, such as motor neurons) as compared to control untreated cells or as compared to pre-administration (in vitro, ex vivo, or in vivo).

### B. Prophylactic or Therapeutic Applications

The CRISPR/Cas systems disclosed herein for targeting a C9orf72 gene and the methods of repressing transcription from a C9orf72 exon 1A transcription start site and/or repressing transcription of sense and/or antisense transcripts that comprise the hexanucleotide repeat expansion sequence in a C9orf72 gene in a cell are useful for the treatment and/or prevention of a C9orf72 hexanucleotide repeat expansion associated disease and/or for ameliorating at least one symptom or indication associated with such disease. In a particular example, the disease is amyotrophic lateral sclerosis (ALS) or frontotemporal dementia (FTD). ALS, also referred to as Lou Gehrig's disease, is the most frequent adult-onset paralytic disorder, characterized by the loss of upper and/or lower motor neurons. ALS occurs in as many as 20,000 individuals across the United States with about 5,000 new cases occurring each year. Frontotemporal dementia (also referred to as Pick's disease, frontotemporal lobar degeneration, or FTLD) is a group of disorders caused by progressive cell degeneration in the frontal or temporal lobes of the brain. FTD is reported to account for 10%-15% of all dementia cases. A hexanucleotide repeat expansion sequence between exon 1A and 1B, two non-coding exons of the human C9ORF72 gene, has been linked to both ALS and FTD. It is estimated that the G₄C₂ hexanucleotide repeat expansion accounts for about 50% of familial and many non-familial ALS cases. It is present in about 25% of familial FTD cases and about 8% of sporadic FTD cases.

Many signs or symptoms related to the hexanucleotide repeat expansion sequence in C9ORF72 have been reported such as, for example, repeat-length-dependent formation of RNA foci, sequestration of specific RNA-binding proteins, and accumulation and aggregation of dipeptide repeat proteins (e.g., poly(glycine-alanine), poly(glycine-proline), poly(glycine-arginine), poly(alanine-proline), or poly(proline-arginine) dipeptide repeat proteins) resulting from repeat-associated non-AUG (AUG) translation.

It is not known how the C9orf72 hexanucleotide repeat expansion causes motor neuron disease and dementia, but two universal postmortem pathological findings in C9orf72 ALS and FTD patients are associated with the repeat expansion: (1) sense and antisense repeat-containing RNA can be visualized as distinct foci in neurons and other cells; and (2) dipeptide repeat proteins—poly(glycine-alanine), poly(glycine-proline), poly(glycine-arginine), poly(alanine-proline), and poly(proline-arginine)—synthesized by repeat-associated non-AUG-dependent translation from the sense and antisense repeat-containing RNAs can be detected in cells. One disease hypothesis proposes that the repeat-containing RNAs, visualized as foci, disrupt cellular RNA metabolism by sequestering RNA binding proteins. Another disease hypothesis posits that the dipeptide repeat proteins exert wide-spread toxic effects on RNA metabolism, proteostasis, and nucleocytoplasmic transport.

Another symptom or indication is the expression of repeat-containing RNAs from the C9orf72 gene. The C9orf72 gene produces transcripts from two transcription initiation sites. The upstream site initiates transcription with alternative non-coding exon 1A, while the downstream site initiates transcription with alternative exon 1B. Both exons 1A and 1B can be spliced to exon 2, which contains the start of the protein-coding sequence. The pathogenic hexanucleotide repeat expansion is located between exons 1A and 1B. Therefore, transcription initiated from exon 1A can produce repeat-containing RNAs, while initiation from exon 1B cannot.

In some methods, a CRISPR/Cas system disclosed herein for targeting a C9orf72 gene may be administered at a therapeutic dose to a subject with a C9orf72 hexanucleotide repeat expansion associated disease. Such methods can comprise administering to a subject a therapeutically effective amount of the CRISPR/Cas system to the subject. A CRISPR/Cas system disclosed herein for targeting a C9orf72 gene can also be used for the preparation of a pharmaceutical composition or medicament for treating patients suffering from a C9orf72 hexanucleotide repeat expansion associated disease. Therapeutic or pharmaceutical compositions comprising CRISPR/Cas system disclosed herein for targeting a C9orf72 gene can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311.

Likewise, the methods for repressing transcription from a C9orf72 exon 1A transcription start site and/or repressing transcription of sense and/or antisense transcripts that comprise the hexanucleotide repeat expansion sequence in a C9orf72 gene disclosed herein can be used for treating a C9orf72 hexanucleotide repeat expansion associated disease. Such therapeutic methods can comprise administering a therapeutically effective amount of a pharmaceutical composition comprising a CRISPR/Cas system disclosed herein for targeting a C9orf72 gene to the subject in need thereof. The C9orf72 hexanucleotide repeat expansion associated disease treated can be any disease or condition associated with C9orf72 hexanucleotide repeat expansion. Some such methods prevent, treat, or ameliorate at least one symptom of a C9orf72 hexanucleotide repeat expansion associated disease (described above), the method comprising administering a therapeutically effective amount of a CRISPR/Cas system disclosed herein for targeting a C9orf72 gene to a subject in need thereof. In some methods, the CRISPR/Cas system disclosed herein for targeting a C9orf72 gene may be administered prophylactically or therapeutically to a subject having or at risk of having a C9orf72 hexanucleotide repeat expansion associated disease. The CRISPR/Cas system disclosed herein for targeting a C9orf72 gene may be administered via intracerebroventricular injection, intracranial injection, intrathecal injection, or by any other suitable means.

A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

A subject can be an animal, optionally a mammal, optionally a human, in need of amelioration, prevention, and/or treatment of a C9orf72 hexanucleotide repeat expansion associated disease. The term includes human subjects who have or are at risk of having such a disease.

The terms treat, treating, or treatment refer to the reduction or amelioration of the severity of at least one symptom or indication of a C9orf72 hexanucleotide repeat expansion associated disease due to the administration of a therapeutic agent such as a CRISPR/Cas system disclosed herein for targeting a C9orf72 gene to a subject in need thereof. The terms include inhibition of progression of disease or of worsening of a symptom/indication. The terms also include positive prognosis of disease (i.e., the subject may be free of disease or may have reduced disease upon administration of a therapeutic agent such as a CRISPR/Cas system disclosed herein for targeting a C9orf72 gene). The therapeutic agent may be administered at a therapeutic dose to the subject. The terms prevent, preventing, or prevention refer to inhibition of manifestation of a C9orf72 hexanucleotide repeat expansion associated disease or any symptoms or indications of such a disease upon administration of a CRISPR/Cas system disclosed herein for targeting a C9orf72 gene.

In some methods, a single dose of a CRISPR/Cas system disclosed herein for targeting a C9orf72 gene may be administered to a subject in need thereof. In other methods, multiple doses of a CRISPR/Cas system disclosed herein for targeting a C9orf72 gene may be administered to a subject over a defined time course. Such methods can comprise sequentially administering to a subject multiple doses of a CRISPR/Cas system disclosed herein for targeting a C9orf72 gene. Sequentially administering means that each dose of the CRISPR/Cas system is administered to the subject at a different point in time, such as on different days separated by a predetermined interval (e.g., hours, days, weeks, or months). Some methods comprise sequentially administering to the patient a single initial dose of a CRISPR/Cas system disclosed herein for targeting a C9orf72 gene, followed by one or more secondary doses of the CRISPR/Cas system, and optionally followed by one or more tertiary doses.

Initial dose, secondary doses, and tertiary doses refer to the temporal sequence of administration of the CRISPR/Cas system disclosed herein for targeting a C9orf72 gene. Thus, the initial dose is the dose which is administered at the beginning of the treatment regimen (also referred to as the baseline dose), the secondary doses are the doses which are administered after the initial dose, and the tertiary doses are the doses which are administered after the secondary doses. The initial, secondary, and tertiary doses may all contain the same amount of the CRISPR/Cas system, but generally may differ from one another in terms of frequency of administration. In some methods, however, the amount of the CRISPR/Cas system contained in the initial, secondary, and/or tertiary doses varies from one another (e.g., adjusted up or down as appropriate) during the course of treatment. In some methods, two or more (e.g., 2, 3, 4, or 5) doses are administered at the beginning of the treatment regimen as loading doses followed by subsequent doses that are administered on a less frequent basis (e.g., maintenance doses).

Such methods may comprise administering to a patient any number of secondary and/or tertiary doses of a CRISPR/Cas system disclosed herein for targeting a C9orf72 gene. In one example, only a single secondary dose is administered to the subject. In another example, two or more (e.g., 2, 3, 4, 5, 6, 7, 8, or more) secondary doses are administered to the subject. Likewise, in another example, only a single tertiary dose is administered to the subject. In other examples, two or more (e.g., 2, 3, 4, 5, 6, 7, 8, or more) tertiary doses are administered to the subject.

The frequency at which the secondary and/or tertiary doses are administered to a subject can vary over the course of the treatment regimen. The frequency of administration may also be adjusted during the course of treatment by a physician depending on the needs of the individual subject following clinical examination.

In some methods, the resulting percent expression from the C9orf72 exon 1A transcription start site in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression from the C9orf72 exon 1A transcription start site in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of sense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of sense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of antisense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of antisense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the resulting percent expression of both sense and antisense C9orf72 hexanucleotide-repeat-containing transcripts in treated cells (e.g., neurons, such as motor neurons) as compared to control untreated cells or as compared to pre-administration (in vitro, ex vivo, or in vivo) can be less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, or less than about 25%. The resulting percent expression can be, e.g., at 1 week post-administration, 2 weeks post-administration, 3 weeks post-administration, or 4 weeks post-administration, or any other suitable time.

In some methods, the percent decrease in expression of both sense and antisense C9orf72 hexanucleotide-repeat-containing transcripts in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGA dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGP dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

In some methods, the percent decrease in expression of polyGA and polyGP dipeptide repeat proteins in treated cells (e.g., neurons, such as motor neurons) as compared to control untreated cells or as compared to pre-administration (in vitro, ex vivo, or in vivo) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%. The percent decrease can be, e.g., at 1 week post-administration, 2 weeks post-administration, 3 weeks post-administration, or 4 weeks post-administration, or any other suitable time.

Such methods can, for example, reduce or abolish expression of transcripts that initiate at C9orf72 exon 1A. Such methods can also, for example, reduce or abolish expression of C9orf72 hexanucleotide-repeat-containing transcripts. Such methods can also, for example, reduce or abolish expression of sense C9orf72 hexanucleotide-repeat-containing transcripts. Such methods can also, for example, reduce or abolish expression of antisense C9orf72 hexanucleotide-repeat-containing transcripts. Such methods can also, for example, reduce or abolish expression of both sense and antisense C9orf72 hexanucleotide-repeat-containing transcripts. Such methods can, for example, selectively or preferentially reduce or abolish expression of transcripts that initiate at C9orf72 exon 1A relative to the effect on expression of transcripts that initiate at C9orf72 exon 1B (i.e., reduce expression of transcripts that initiate at C9orf72 exon 1A to a greater extent than reducing expression of transcripts that initiate at C9orf72 exon 1B). Such methods can also, for example, selectively or preferentially reduce or abolish expression of C9orf72 hexanucleotide-repeat-containing transcripts relative to the effect on expression of transcripts that initiate at C9orf72 exon 1B (i.e., reduce expression of C9orf72 hexanucleotide-repeat-containing transcripts to a greater extent than reducing expression of transcripts that initiate at C9orf72 exon 1B). Such methods can also, for example, selectively or preferentially reduce or abolish expression of sense C9orf72 hexanucleotide-repeat-containing transcripts relative to the effect on expression of transcripts that initiate at C9orf72 exon 1B (e.g., reduce expression of sense C9orf72 hexanucleotide-repeat-containing transcripts to a greater extent than reducing expression of transcripts that initiate at C9orf72 exon 1B). Such methods can also, for example, selectively or preferentially reduce or abolish expression of antisense C9orf72 hexanucleotide-repeat-containing transcripts relative to the effect on expression of transcripts that initiate at C9orf72 exon 1B (e.g., reduce expression of antisense C9orf72 hexanucleotide-repeat-containing transcripts to a greater extent than reducing expression of transcripts that initiate at C9orf72 exon 1B). Such methods can also, for example, selectively or preferentially reduce or abolish expression of both sense and antisense C9orf72 hexanucleotide-repeat-containing transcripts relative to the effect on expression of transcripts that initiate at C9orf72 exon 1B (e.g., reduce expression of both sense and antisense C9orf72 hexanucleotide-repeat-containing transcripts to a greater extent than reducing expression of transcripts that initiate at C9orf72 exon 1B). In some methods, the CRISPR/Cas system reduces or abolishes expression of transcripts that initiate at C9orf72 exon 1A but does not reduce or abolish expression of transcripts that initiate at C9orf72 exon 1B. In some methods, the CRISPR/Cas system reduces or abolishes expression of C9orf72 hexanucleotide-repeat-containing transcripts but does not reduce or abolish expression of transcripts that initiate at C9orf72 exon 1B. In some methods, the CRISPR/Cas system reduces or abolishes expression of sense C9orf72 hexanucleotide-repeat-containing transcripts but does not reduce or abolish expression of transcripts that initiate at C9orf72 exon 1B. In some methods, the CRISPR/Cas system reduces or abolishes expression of antisense C9orf72 hexanucleotide-repeat-containing transcripts but does not reduce or abolish expression of transcripts that initiate at C9orf72 exon 1B. In some methods, the CRISPR/Cas system reduces or abolishes expression of both sense and antisense C9orf72 hexanucleotide-repeat-containing transcripts but does not reduce or abolish expression of transcripts that initiate at C9orf72 exon 1B. In some methods disclosed herein, the CRISPR/Cas system reduces expression of polyGA dipeptide repeat proteins. In some methods disclosed herein, the CRISPR/Cas system reduces expression of polyGP dipeptide repeat proteins. In some methods disclosed herein, the CRISPR/Cas system reduces expression of both polyGA dipeptide repeat proteins and polyGP dipeptide repeat proteins.

Some methods result in a decrease in sense and/or antisense repeat-containing RNA foci in treated cells (e.g., neurons, such as motor neurons) as compared to control untreated cells or as compared to pre-administration (in vitro, ex vivo, or in vivo). Some methods result in a decrease in dipeptide repeat proteins (e.g., poly(glycine-alanine), poly(glycine-proline), poly(glycine-arginine), poly(alanine-proline), and/or poly(proline-arginine)) synthesized by repeat-associated non-AUG-dependent translation from the sense and antisense repeat-containing RNAs in treated cells (e.g., neurons, such as motor neurons) as compared to control untreated cells or as compared to pre-administration (in vitro, ex vivo, or in vivo).

Methods for measuring expression of transcripts that initiate at C9orf72 exon 1A, expression of transcripts that initiate at C9orf72 exon 1B, and expression of sense and antisense C9orf72 hexanucleotide-repeat-containing transcripts are known and are described elsewhere herein. Assessment of expression (or measuring RNA foci and dipeptide repeats as described elsewhere herein) can be in any cell type (e.g., neurons, such as motor neurons).

The prophylactic or therapeutic methods described herein can be combined with other therapeutic or prophylactic treatments for C9orf72 hexanucleotide repeat expansion associated diseases.

### C. Administering CRISPR/Cas Systems to Animals or Cells

The methods disclosed herein can comprise introducing into an animal (e.g., mammal, such as a human) or cell various CRISPR/Cas systems targeting C9orf72, including in the form of nucleic acids (e.g., DNA or RNA), proteins, or nucleic-acid-protein complexes. “Introducing” includes presenting to the cell or animal the molecule(s) (e.g., nucleic acid(s) or protein(s)) in such a manner that it gains access to the interior of the cell or to the interior of cells within the animal. The introducing can be accomplished by any means, and two or more of the components (e.g., two of the components, or all of the components) can be introduced into the cell or animal simultaneously or sequentially in any combination. For example, a Cas protein can be introduced into a cell or animal before introduction of a guide RNA, or it can be introduced following introduction of the guide RNA. See, e.g., US 2015/0240263 and US 2015/0110762, each of which is herein incorporated by reference in its entirety for all purposes. In addition, two or more of the components can be introduced into the cell or animal by the same delivery method or different delivery methods. Similarly, two or more of the components can be introduced into an animal by the same route of administration or different routes of administration.

A CRISPR/Cas system can be introduced into an animal or cell or one or more nucleic acids encoding the CRISPR/Cas system can be introduced into the cell. A guide RNA can be introduced into an animal or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the animal. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or an animal, the Cas protein can be transiently, conditionally, or constitutively expressed in the cell or in a cell in the animal.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a neuron, such as a motor neuron. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery.

Molecules (e.g., Cas proteins or guide RNAs) introduced into the animal or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or animal. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is optionally into the cytoplasm (e.g., to deliver mRNA directly to the translation machinery), while microinjection of a Cas protein or a polynucleotide encoding a Cas protein or encoding an RNA is optionally into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein optionally comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to an animal include hydrodynamic delivery, virus-mediated delivery (e.g., adeno-associated virus (AAV)-mediated delivery), and lipid-nanoparticle-mediated delivery.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes, a gene expression cassette can be placed between ITR sequences. Typically, rAAV genome cassettes comprise of a promoter to drive expression of a transgene, followed by a polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas protein, such as Cas9, and DNA encoding one or more guide RNAs (e.g., DNA encoding a guide RNA, or DNA encoding two or more guide RNAs).

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery. For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Introduction of nucleic acids and proteins can also be accomplished by lipid nanoparticle (LNP)-mediated delivery. The lipid nanoparticles can comprise the CRISPR/Cas systems disclosed herein. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. See, e.g., WO 2016/010840 A1 and WO 2017/173054 A1, each of which is herein incorporated by reference in its entirety for all purposes. An exemplary lipid nanoparticle can comprise a cationic lipid and one or more other components.

In some LNPs, the cargo can comprise Cas mRNA (e.g., Cas9 mRNA) and gRNA. The Cas mRNA and gRNAs can be in different ratios.

Examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, or gRNA or nucleic acid encoding). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas protein being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. For example, administration may be directly to the brain of a subject or to neurons in the brain of a subject. In a specific example, administration to a subject is by intrathecal injection or by intracranial injection (e.g., stereotactic surgery for injection in the hippocampus and other brain regions, or intracerebroventricular injection). In a specific example, administration to a subject is by intracerebroventricular injection. In another specific example, administration to a subject is by intracranial injection. In another specific example, administration to a subject is by intrathecal injection.

In some methods, administration is by a means such that the reagent being introduced reaches neurons or the nervous system. This can be achieved, for example, by peripheral delivery or by direct delivery to the nervous system. See, e.g., Evers et al. (2015) Adv. Drug Deliv. Res. 87:90-103, herein incorporated by reference in its entirety for all purposes.

For reagents to reach the nervous system, they first have to cross the vascular barrier, made up of the blood brain barrier or the blood-spinal cord barrier. One mechanism that can be used to cross the vascular barrier is receptor-mediated endocytosis. Another mechanism that can be used is cell-penetrating peptide (CPP)-based delivery systems. Different CPPs use distinct cellular translocation pathways, which depend on cell types and cargos. Another delivery mechanism that can be used is exosomes, which are extracellular vesicles known to mediate communication between cells through transfer of proteins and nucleic acids. For example, IV injection of exosomes transduced with short viral peptides derived from rabies virus glycoprotein (RVG) can result in crossing of the blood brain barrier and delivery to the brain.

Techniques are also available that bypass the vascular barriers through direct infusion into the cerebrospinal fluid. For example, reagents can be infused intracerebroventricularly (ICV), after which the reagents would have to pass the ependymal cell layer that lines the ventricular system to enter the parenchyma. Intrathecal (IT) delivery means delivery of the reagents into the subarachnoid space of the spinal cord. From here, reagents will have to pass the pia mater to enter the parenchyma. Reagents can be delivered ICT or IT through an outlet catheter that is connected to an implanted reservoir. Drugs can be injected into the reservoir and delivered directly to the CSF. Intranasal administration is an alternative route of delivery that can be used.

The frequency of administration and the number of dosages can depend on the half-life of the CRISPR/Cas system components (e.g., guide RNAs, Cas proteins (or nucleic acids encoding the guide RNAs or Cas proteins)) and the route of administration among other factors. The introduction of nucleic acids or proteins into the cell or animal can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

### IV. Cells or Animals

Cells or subjects (e.g., animals) comprising the CRISPR/Cas systems (or components thereof), vectors, or lipid nanoparticles disclosed herein are also provided.

The cells or subjects can be, for example, mammalian, non-human mammalian, and human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans.

The cells can be isolated cells (e.g., in vitro) or can be in vivo within a subject (e.g., animal or mammal). Cells can also be any type of undifferentiated or differentiated state. In one example, the cells are neurons.

The cells provided herein can be normal, healthy cells, or can be diseased cells comprising expanded hexanucleotide repeats at the C9orf72 locus as described elsewhere herein.

In one example, the cell is a human cell, a rodent cell, a mouse cell, or a rat cell such as a human neuron, a rodent neuron, a mouse neuron, or a rat number. In a specific example, the cell is a human neuron. In a specific example, the cell is in vivo in a subject (e.g., a neuron in the brain of a subject).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ | | |
|---|---|---|
| ID NO | Type | Description |
| 1 | Protein | Cas9 Protein |
| 2 | DNA | Cas9 CDS |
| 3 | Protein | SV40 NLS v1 |
| 4 | Protein | SV40 NLS v2 |
| 5 | Protein | Nucleoplasmin NLS |
| 6 | RNA | crRNA Tail v1 |
| 7 | RNA | crRNA Tail v2 |
| 8 | RNA | TracrRNA v1 |
| 9 | RNA | TracrRNA v2 |
| 10 | RNA | TracrRNA v3 |
| 11 | RNA | gRNA Scaffold v1 |
| 12 | RNA | gRNA Scaffold v2 |
| 13 | RNA | gRNA Scaffold v3 |
| 14 | RNA | gRNA Scaffold v4 |
| 15 | RNA | gRNA Scaffold v5 |
| 16 | RNA | gRNA Scaffold v6 |
| 17 | RNA | gRNA Scaffold v7 |
| 18 | RNA | gRNA Scaffold v8 |
| 19 | DNA | Guide RNA Target Sequence Plus PAM v1 |
| 20 | DNA | Guide RNA Target Sequence Plus PAM v2 |
| 21 | DNA | Guide RNA Target Sequence Plus PAM v3 |
| 22 | DNA | Mus musculus C9orf72 mRNA (NM_001081343.1) |
| 23 | Protein | Mus musculus C9orf72 Amino Acid |
| | | (NP_001074812.1) |
| 24 | DNA | Rattus norvegicus C9orf72 mRNA |
| | | (NM_001007702.1) |
| 25 | Protein | Rattus norvegicus C9orf72 Amino Acid |
| | | (NP_001007703.1) |
| 26 | DNA | Homo sapiens C9orf72 mRNA (NM_145005.6) |
| 27 | Protein | Homo sapiens C9orf72 Amino Acid (NP_659442.2) |
| 28 | DNA | Homo sapiens C9orf72 mRNA (NM_018325.4) |
| 29 | Protein | Homo sapiens C9orf72 Amino Acid (NP_060795.1) |
| 30 | DNA | Homo sapiens C9orf72 mRNA (NM_001256054.2) |
| 31 | Protein | Homo sapiens C9orf72 Amino Acid |
| | | (NP_001242983.1) |
| 32-51 | DNA | Mouse C9orf72 gRNA Promoter Target Sequences |
| 52-71 | DNA | Human C9orf72 gRNA Promoter Target Sequences |
| 72-91 | RNA | Mouse C9orf72 gRNA Promoter DNA-Targeting |
| | | Segments |
| 92-111 | RNA | Human C9orf72 gRNA Promoter DNA-Targeting |
| | | Segments |
| 112 | DNA | Human C9orf72 gRNA Target Sequence hGU21 |
| 113 | RNA | Human C9orf72 gRNA DNA-Targeting Segment |
| | | hGU21 |
| 114-117 | DNA | C9orf72 Hexanucleotide Repeat gRNA Target |
| | | Sequences |
| 118-121 | RNA | C9orf72 Hexanucleotide Repeat gRNA DNA- |
| | | Targeting Segments |
| 122-133 | DNA | Primer and Probes |
| 134 | DNA | Alt-R sgRNA Target Sequence |

### EXAMPLES

### Example 1. CRISPR Interference of C9Orf72 Repeat Expansion Alleles

Amyotrophic lateral sclerosis (ALS) and frontotemporal lobar degeneration (FTLD) are progressive and fatal neurodegenerative diseases that cause motor neuron disease in the case of ALS and dementia in the case of FTLD. The most common cause of familial ALS is an expansion of a GGGGCC (G₄C₂) hexanucleotide repeat between two alternative 5′ non-coding exons of the C9orf72 gene. Normal individuals have between 3 and 35 G₄C₂ repeats, while ALS or FTLD patients harbor repeat numbers in the hundreds or thousands. The physiological function of the C9orf72 protein is not well understood, and no disease-causing mutations have been identified in its coding sequence. The G₄C₂ repeats are transcribed in a bidirectional manner, resulting in both G₄C₂ (sense with respect to the C9orf72 mRNA) and G₂C₄ (antisense) repeat-containing transcripts, which are translated into six different dipeptide repeat (DPR) proteins by a non-canonical mechanism termed Repeat Associated Non-AUG (RAN) translation. Both the repeat-containing RNAs and the DPR proteins derived from them have been implicated as pathogenic agents in C9orf72 ALS and FTLD. Although the exact mechanisms by which repeat-containing RNA and DPR proteins promote the disease state are not known, it appears clear that synthesis of RNA from the repeat expansion is a pre-requisite for pathogenesis.

Transcription of the C9orf72 gene initiates at two alternative non-coding exons: exon 1A (upstream) and exon 1B (downstream). The G₄C₂ repeat lies between exons 1A and 1B. Exons 1A and 1B can be spliced to exon 2, the first protein-coding exon, creating mRNAs with alternative 5′-untranslated regions. In healthy people with short G₄C₂ repeat expansions, transcription predominantly initiates at exon 1B; RNAs that include exon 1A are rare, and repeat-containing RNAs are undetectable. People suffering from C9orf72 ALS or FTLD accumulate transcripts in which exon 1A is spliced to exon 2, and both sense and antisense repeat-containing RNAs and the DPR proteins translated from them can be detected by in situ hybridization and immunohistochemistry. See FIG. 1. These pathological findings suggest that the longer disease-associated G₄C₂ repeat expansions promote the use of the upstream exon 1A transcription initiation site, which is the only way that repeat-containing RNAs and their DPR proteins could be produced. It also follows that the production of antisense repeat-containing RNA, which depends on a long repeat expansion, is somehow linked to increased use of the upstream transcription initiation site.

Thus, a possible therapeutic strategy for C9orf72 repeat-expansion disease would be to inhibit or abolish transcription that initiates upstream of the G₄C₂ repeat at exon 1A while retaining transcription that initiates at exon 1b downstream of the repeat, which will retain production of the mRNA for C9orf72 protein synthesis. Under the assumption that there are promoter elements upstream of exon 1A that control transcription of the C9orf72 gene, we designed experiments to determine if transcripts that initiate at exon 1A could be selectively reduced while retaining transcripts that initiate at exon 1b.

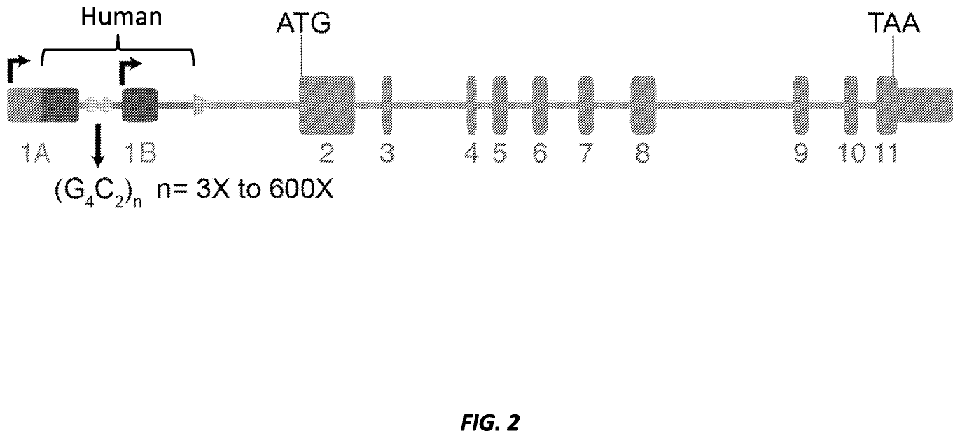

To test this, we used mouse embryonic stem (ES) cells in which we placed a fragment from the human C9orf72 gene, including part of exon 1A, the intron sequence between 1A and 1B, including 300 G₄C₂ repeats (300×), all of exon 1B and part of the downstream intron, precisely at its homologous position in one allele of the mouse C9orf72 gene. See FIG. 2. See also US 2020-0196581 and WO 2020/131632, each of which is herein incorporated by reference in its entirety for all purposes. As a reliable model of C9orf72 repeat expansion disease, the 300× humanized allele reproduces the molecular hallmarks of disease: a switch in transcription initiation from exon 1B to exon 1A; accumulation of sense and antisense repeat-containing RNA; production of poly-Glycine-Alanine and poly-Glycine-Proline DPR proteins; accumulation of intron-containing sense RNAs; and accumulation of antisense RNAs that initiate approximately 170 bp downstream of exon 1B and extend through the G₄C₂ repeat and into flanking sequences upstream of exon 1A.

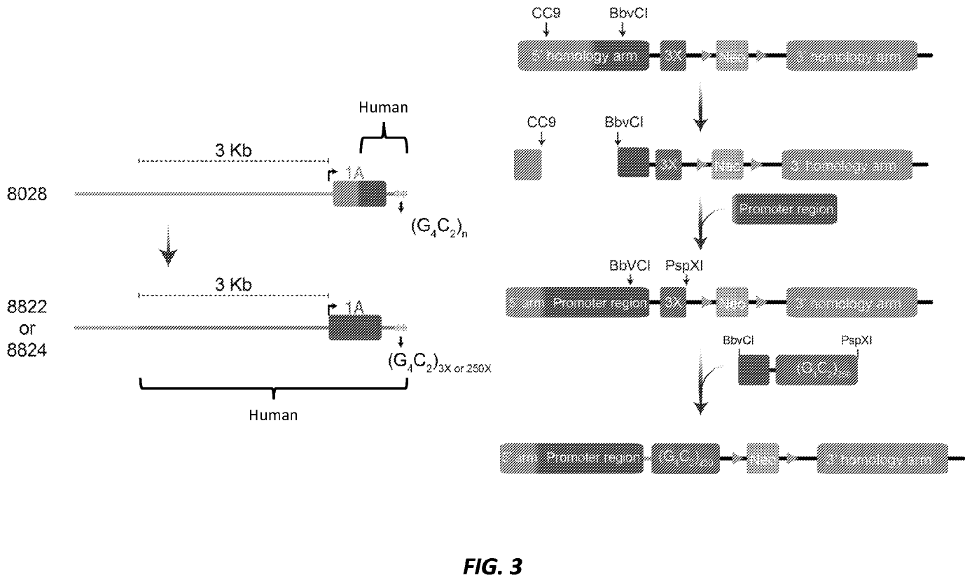

We also generated an extended mouse humanized C9orf72 repeat expansion model in which the humanization extends to include approximately 3 kb of flanking sequence upstream of exon 1A in order to repeat the CRISPR/Cas9 screening using gRNAs targeting the human C9orf72 promoter region. A schematic for generating the C9orf72 allele with the humanized exon 1A promoter is shown in FIG. 3.

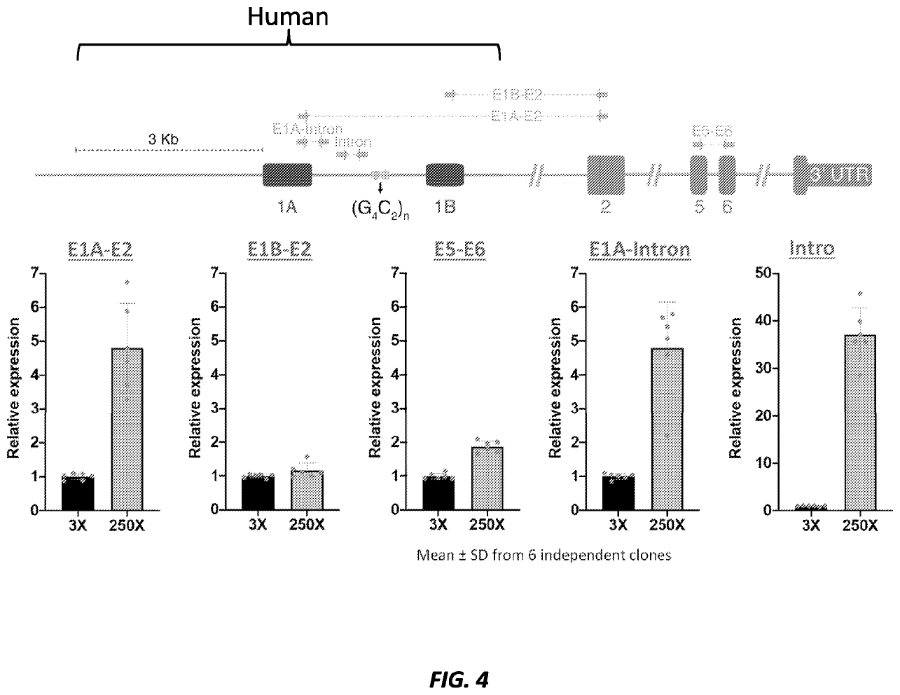

To validate the allele with the humanized C9orf72 promoter, we assessed the expression of transcripts in mouse ES cells having the allele with the humanized C9orf72 promoter and either 3× repeats or 250× repeats through use of TAQMAN® qualitative PCR assays spanning different sections of the C9orf72 pre-mRNA. FIG. 4 shows that the allele with the humanized C9orf72 promoter region reproduces the RNA expression pattern seen in repeat expansion alleles with the mouse promoter, with exon 1A and intron-containing transcripts being expressed at higher levels in 250× repeat mouse ES cells as compared to 3× repeat mouse ES cells, and exon 1B transcripts being expressed at similar levels in 250× repeat mouse ES cells as compared to 3× repeat mouse ES cells.

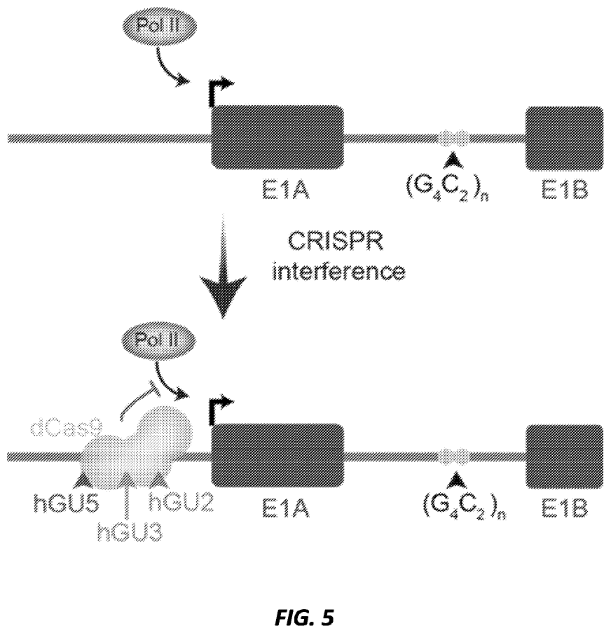

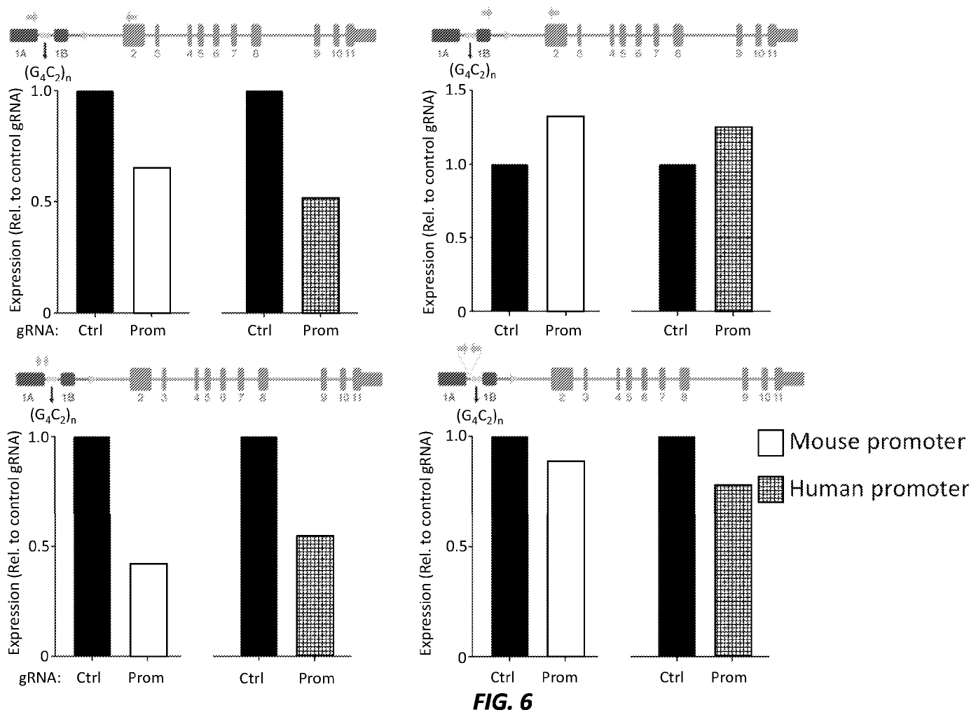

Using these mouse ES cells, we used the Clustered Regularly Interspaced Short Palindromic Repeats (CRISPR)/Cas9 DNA targeting technology with a catalytically dead version of the Cas9 enzyme (dCas9) and sequence-specific guide RNAs (gRNAs) to substantially reduce or eliminate transcription that initiates at exon 1A while retaining transcription that initiates at exon 1B. We designed two independent approaches. In the first approach, gRNAs target specific DNA sequences to direct the binding of dCas9 to the promoter region upstream of exon 1A. See FIG. 5. As shown in FIG. 6, such gRNAs target specific DNA sequences to direct the binding of dCas9 to the promoter region upstream of exon 1A in a manner that disrupts transcription initiation from the exon 1A start site while preserving transcription initiation from the exon 1B start site. Specifically, we have shown in the 300×G₄C₂ repeat expansion humanized mouse ES cells and mouse ES cells with the allele with the humanized C9orf72 promoter and 250× G₄C₂ repeats that promoting dCas9 binding to a region spanning 200 bp upstream of exon 1A with one guide RNA (mGU3) or a collection of three guide RNAs (hGU2, hGU3, and hGU4) caused a substantial inhibition of transcription initiated at exon 1A while sparing transcripts that initiated at exon 1B.

TABLE 3
Guide RNAs targeting mouse C9orf72 promoter or near E1A Start Site.

| | | | | | Distance from |
|---|---|---|---|---|---|
| | Guide RNA Target | | | | E1A Start Site |
| gRNA | Sequence (SEQ ID NO) | PAM | Location | Strand | (UCSC annotated) |
| mGU | CTTTCCTTCTAGGTGGAAAG | TGG | chr4: 35, 173, 321-35, 173, | | (−)49 bp |
| | (SEQ ID NO: 32) | | 343 | | |
| mGU2 | CGTCTCTGCTTGACGCGCCG | CGG | chr4: 35, 173, 396-35, | Minus | *43 bp |
| | (SEQ ID NO: 33) | | 173, 418 | | |
| mGU3 | AAGCGCCAACCCGGAACTTA | CGG | chr4: 35, 173, 434-35, | | 64 bp |
| | (SEQ ID NO: 34) | | 173, 456 | | |
| mGU4 | AGCCCATGACAGCCTCCGCC | TGG | chr4: 35, 173, 498-35, | | 128 bp |
| | (SEQ ID NO: 35) | | 173, 520 | | |
| mGU5 | GTAACCTAGATAGATAACGC | AGG | chr4: 35, 173, 595-35, | | 225 bp |
| | (SEQ ID NO: 36) | | 173, 617 | | |
| mGU6 | ACGGCATTTAACCGTGTCAT | AGG | chr4: 35, 173, 724-35, | | 354 bp |
| | (SEQ ID NO: 37) | | 173, 746 | | |
| mGU7 | AACACGGTGACTACGCACAG | AGG | chr4: 35, 173, 804-35, | | 434 bp |
| | (SEQ ID NO: 38) | | 173, 826 | | |
| mGU8 | AATCAATAAATCAGACGTCA | TGG | chr4: 35, 173, 895-35, | | 525 bp |
| | (SEQ ID NO: 39) | | 173, 917 | | |
| mGU9 | AGTTCTATTTTTACCGAAGG | TGG | chr4: 35, 174, 042-35, | | 672 bp |
| | (SEQ ID NO: 40) | | 174, 064 | | |
| mGU10 | TCTTACAGTGTTTTGTACCG | TGG | chr4: 35,174, 148-35, | Minus | 795 bp |
| | (SEQ ID NO: 41) | | 174, 170 | | |
| mGU11 | AAATAGCATAGGACCATGTT | TGG | chr4: 35, 174, 237-35, | Minus | 884 bp |
| | (SEQ ID NO: 42) | | 174, 259 | | |
| mGU12 | GTCAGCTAATCCACAACACG | AGG | chr4: 35, 174, 337-35, | Minus | 984 bp |
| | (SEQ ID NO: 43) | | 174, 359 | | |
| mGU13 | ATCAATTACATTGATAGCTC | TGG | chr4: 35, 174, 482-35, | | 1112 bp |
| | (SEQ ID NO: 44) | | 174, 504 | | |
| mGU14 | TGTTAGTTTGACTCACAGAC | AGG | chr4: 35, 174, 590-35, | | 1220 bp |
| | (SEQ ID NO: 45) | | 174, 612 | | |
| mGU15 | GTCTCTAGGTAAAATTTTGA | AGG | chr4: 35, 174, 678-35, | | 1308 bp |
| | (SEQ ID NO: 46) | | 174, 700 | | |
| mGU16 | CTAAAATATTGTATGACCAC | AGG | chr4: 35, 174, 909-35, | Minus | 1556 bp |
| | (SEQ ID NO: 47) | | 174, 931 | | |
| mGU17 | TGGAACAATTACTTAGGCAT | AGG | chr4: 35, 175, 002-35, | Minus | 1649 bp |
| | (SEQ ID NO: 48) | | 175, 024 | | |
| mGU18 | TAATTGTTGTATTGCCTGAA | AGG | chr4: 35, 175, 107-35, | Minus | 1754 bp |
| | (SEQ ID NO: 49) | | 175, 129 | | |
| mGU19 | AATCTACCTCAAATAACTCC | AGG | chr4: 35, 175, 255-35, | Minus | 1902 bp |
| | (SEQ ID NO: 50) | | 175, 277 | | |
| mGU20 | GGAGTAAACCAGTACGTGGT | AGG | chr4: 35, 175, 353-35, | | 1983 bp |
| | (SEQ ID NO: 51) | | 175, 375 | | |

*Depending on the annotation used, the guide RNA target sequence is considered upstream or downstream of the E1A start site.

TABLE 4
Guide RNAs targeting human C9orf72 promoter or near E1A Start Site.

| | Guide RNA Target | | | | Distance from |
|---|---|---|---|---|---|
| Name | Sequence (SEQ ID NO) | PAM | Location | Strand | E1A Start Site |
| hGU21 | AAACAGACAGACGTAACCTA | CGG | | plus | (−)10 bp |
| | (SEQ ID NO: 112) | | | | |
| hGU | TGCGATGACGTTTTCTCACG | AGG | chr9: 27573881-27573903 | minus | 36 bp |
| | (SEQ ID NO: 52) | | | | |
| hGU2 | CTCACGAGGCTAGCGAAATG | GGG | chr9: 27573895-27573917 | minus | 50 bp |
| | (SEQ ID NO: 53) | | | | |
| hGU3 | GCGCCAACGCTCCTCCAGAG | CGG | chr9: 27573952-27573974 | | 90 bp |
| | (SEQ ID NO: 54) | | | | |
| hGU4 | TGTGCGAACCTTAATAGGGG | AGG | chr9: 27573982-27574004 | minus | 137 bp |
| | (SEQ ID NO: 55) | | | | |
| hGU5 | GGCGCTCAACACATAATTGG | TGG | chr9: 27574076-27574098 | minus | 231 bp |
| | (SEQ ID NO: 56) | | | | |
| hGU6 | ACCCTGGTAGGGGACAGCTC | CGG | chr9: 27574156-27574178 | minus | 311 bp |
| | (SEQ ID NO: 57) | | | | |
| hGU7 | CAAGCATGATTTTCACACTG | GGG | chr9: 27574250-27574272 | minus | 405 bp |
| | (SEQ ID NO: 58) | | | | |
| hGU8 | CCTGCAGACCAAAAGACGCA | AGG | chr9: 27574378-27574400 | | 516 bp |
| | (SEQ ID NO: 59) | | | | |
| hGU9 | TTTGATCTGCAAGTGTGAGA | TGG | chr9: 27574486-27574508 | minus | 641 bp |
| | (SEQ ID NO: 60) | | | | |
| hGU10 | AACGTTTTAATCATTCACCG | AGG | chr9: 27574562-27574584 | minus | 717 bp |
| | (SEQ ID NO: 61) | | | | |
| hGU11 | TTCACAAAGCATAGAACAGT | AGG | chr9: 27574654-27574676 | | 792 bp |
| | (SEQ ID NO: 62) | | | | |
| hGU12 | ATACCCAACTAAAATAGTGG | GGG | chr9: 27574708-27574730 | minus | 863 bp |
| | (SEQ ID NO: 63) | | | | |
| hGU13 | ACTTGGAGAGAATTACACTG | TGG | chr9: 27574770-27574792 | | 908 bp |
| | (SEQ ID NO: 64) | | | | |
| hGU14 | TTGGCACTATTAAGGATCTG | AGG | chr9: 27574913-27574935 | | 1051 bp |
| | (SEQ ID NO: 65) | | | | |
| hGU15 | GGGCATACATGTAGATCAGA | TGG | chr9: 27575021-27575043 | | 1159 bp |
| | (SEQ ID NO: 66) | | | | |
| hGU16 | AGTTGTACTTCACTTATACG | TGG | chr9: 27575159-27575181 | minus | 1314 bp |
| | (SEQ ID NO: 67) | | | | |
| hGU17 | CAGAGTAGACCCTTGGTTGG | GGG | chr9: 27575210-27575232 | minus | 1365 bp |
| | (SEQ ID NO: 68) | | | | |
| hGU18 | GGGAGACCCCAGACTTCACA | TGG | chr9: 27575494-27575516 | minus | 1649 bp |
| | (SEQ ID NO: 69) | | | | |
| hGU19 | TGGCTTTCAGACTTGCAATG | GGG | chr9: 27576007-27576029 | | 2145 bp |
| | (SEQ ID NO: 70) | | | | |
| hGU20 | TCTCCTTTGACTCACACCCA | GGG | chr9: 27576066-27576088 | minus | 2221 bp |
| | (SEQ ID NO: 71) | | | | |

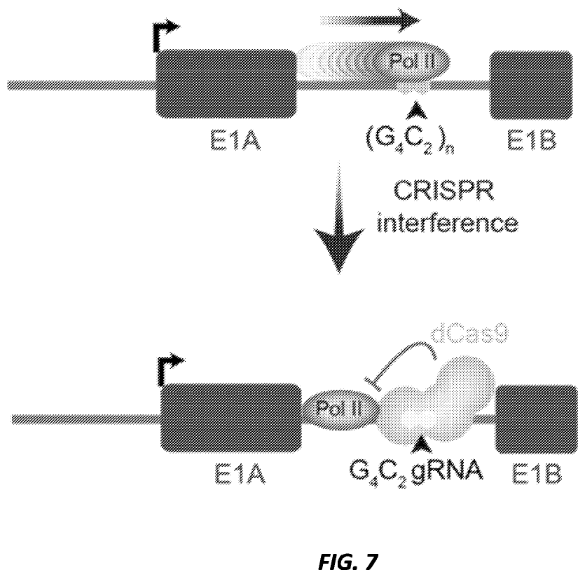

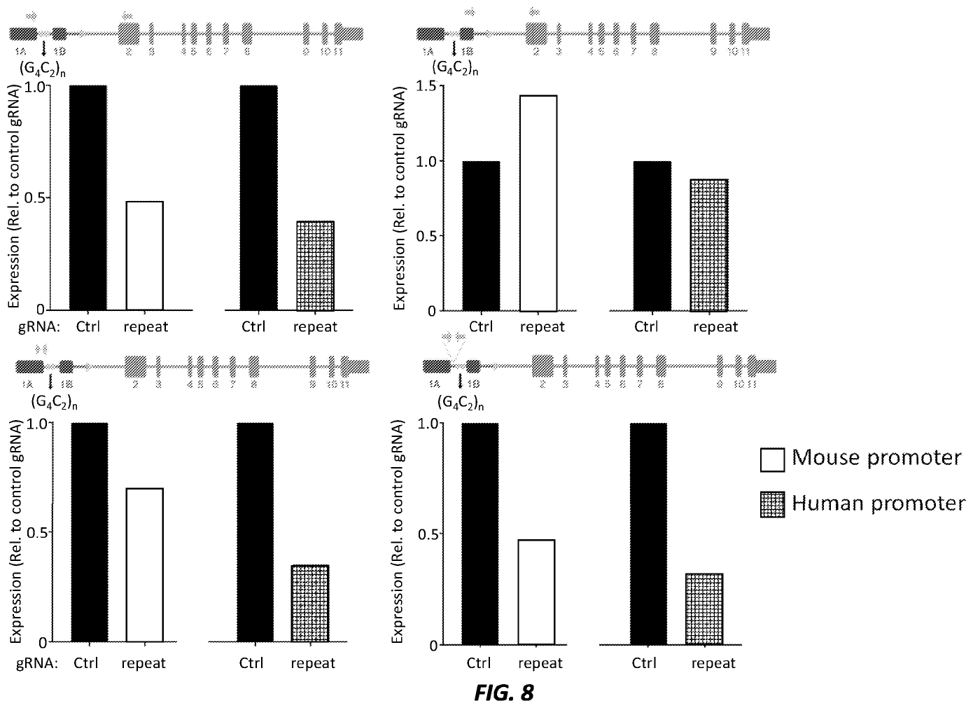

In the second approach, gRNAs against the G₄C₂ repeats directed dCas9 binding to the repeat sequence, thereby coating the repeat with multiple dCas9 proteins. See FIG. 7. As shown in FIG. 8, gRNAs against the G₄C₂ repeats directed dCas9 binding to the repeat sequence hindered transcription elongation through the repeat and substantially reduced the accumulation of RNAs that initiated at exon 1A without affecting transcription from exon 1B. Specifically, we have shown in the 300×G₄C₂ repeat expansion humanized mouse ES cells and mouse ES cells with the allele with the humanized C9orf72 promoter and 250× G₄C₂ repeats that promoting dCas9 binding to the hexanucleotide repeat region caused a substantial inhibition of transcription initiated at exon 1A while sparing transcripts that initiated at exon 1B.

TABLE 5
Guide RNAs targeting hexanucleotide repeat.

| | Guide RNA Target | | SEQ |
|---|---|---|---|
| Name | Sequence | PAM | ID NO |
| 1 | GCCGGGGCCGGGGCCGGGGC | CGG | 114 |
| 2 | CCGGGGCCGGGGCCGGGGCC | GGG | 115 |
| 3 | CGGGGCCGGGGCCGGGGCCG | GGG | 116 |
| 4 | CCCGGCCCCGGCCCCGGCCC | CGG | 117 |

When applying both approaches, we have not observed any discernable effect on the abundance of the C9orf72 mRNA. The data showed that methods that direct catalytically dead dCas9 enzyme to act as a physical block for RNA transcription initiation or elongation reduce or abolish the synthesis of pathogenic RNAs. These methods differ from the current standard method of CRISPR-induced transcription inhibition, in which gRNAs direct a dCas9 with a covalently attached transcription repression domain, such as KRAB, to promoter sequences to repress transcription of the targeted gene.

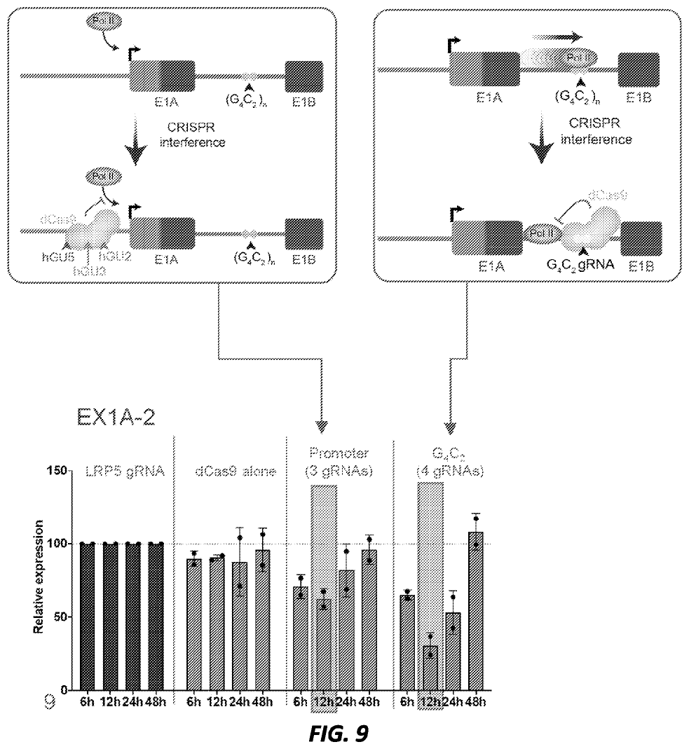

In a confirmatory experiment, 300×G₄C₂ repeat expansion humanized mouse ES cells were electroporated with dCas9 protein along with gRNAs against either the promoter region (3 gRNAs: mGU3, mGU4, and mGU5) or the G₄C₂ repeats (4 gRNAs from Table 5). dCas9 alone or dCas9 plus a negative control gRNA targeting LRP5 were used as negative controls. Specifically, dCas9 RNP complexes were formed by combining 31.25 pmol of dCas9 protein (IDT #1081067) per 125 pmol of each sgRNA (IDT) for 15 minutes at room temperature before mixing with Lonza P3 buffer. dCas9 RNPs were delivered into ES cells that contain a 300×G₄C₂ repeat expansion using the Lonza 4D nucleofector instrument. Following nucleofection, ES cells were plated onto a gelatin-coated 24-well plate at 1 Million, 500K, 250K and 150K cells/well for growth and collected for RNA analysis 6, 12, 24 and 48 hours after nucleofection. At the indicated timepoints after electroporation, ES cells were harvested. Total RNA was isolated using RNeasy plus kit (Qiagen) followed by DNase treatment using the Turbo DNA free kit (Thermo Fisher Scientific). RT-qPCR analysis was performed in a one-step reaction with QuantiNova Probe RT-PCR kit (Qiagen) according to the manufacturer's recommendations. The RT-qPCR contained 2 μL of RNA and an 8 μL of a mixture containing RT-PCR Master mix, ROX dye, RT-mix, and custom-designed primer-probe mix. The custom TaqMan assays were obtained from IDT and the reference assay for Drosha mRNA from ThermoFisher (cat. #Mm01310009_ml). We performed the qRT-PCR reactions in quadruplicate on a ViiA™ 7 Real-Time PCR Detection System (ThermoFisher) with the RT reaction at 45° C. for 10 min followed by PCR cycling (95° C. 10 min, 45 cycles of 95° C. 5 sec, 60° C. 30 s) in an optical 384-well plate. We determined ΔCt values between the target RNA assays and the Drosha mRNA reference. We observed robust, transient loss of pathogenic RNA transcripts but no effect on the abundance of the C9orf72 mRNA. As shown in FIG. 9, gRNAs targeting dCas9 to either the promoter or the G₄C₂ repeat cause a reduction in transcription initiation or elongation through the repeats. Although this experiment used 4 different gRNAs targeting the G₄C₂ repeats and 3 gRNAs targeting the promoter region, similar results can be achieved using 1, 2, or 3 gRNAs.

In a further confirmatory experiment, motor neurons derived from 300×G₄C₂ repeat expansion humanized mouse ES cells (ESMNs) are infected with lentivirus encoding dCas9 protein and gRNAs against either the promoter region (e.g., 3 gRNAs: mGU3, mGU4, and mGU5) or the G₄C₂ repeats (e.g., 4 gRNAs from Table 5). As with the ES cell experiments, at various timepoints after infection, ES cells are harvested. Total RNA is isolated using RNeasy plus kit (Qiagen) followed by DNase treatment using the Turbo DNA free kit (Thermo Fisher Scientific). RT-qPCR analysis is performed in a one-step reaction with QuantiNova Probe RT-PCR kit (Qiagen) according to the manufacturer's recommendations. The RT-qPCR contains 2 μL of RNA and an 8 μL of a mixture containing RT-PCR Master mix, ROX dye, RT-mix, and custom-designed primer-probe mix. The custom TaqMan assays are obtained from IDT and the reference assay for Drosha mRNA from ThermoFisher (cat. #Mm01310009_ml). We perform the qRT-PCR reactions in quadruplicate on a ViiA™ 7 Real-Time PCR Detection System (ThermoFisher) with the RT reaction at 45° C. for 10 min followed by PCR cycling (95° C. 10 min, 45 cycles of 95° C. 5 sec, 60° C. 30 s) in an optical 384-well plate. We determine ΔCt values between the target RNA assays and the Drosha mRNA reference.

To reproduce the preferential ablation of exon 1A transcripts in mice, C9orf72 G₄C₂₃₀₀× heterozygous mice (described in US 2020-0196581 and WO 2020/131632, each of which is herein incorporated by reference in its entirety for all purposes) are used. Nucleic acids encoding dCas9 and gRNAs against either the promoter region (e.g., 3 gRNAs: mGU3, mGU4, and mGU5) or the G₄C₂ repeats (e.g., 4 gRNAs from Table 5) (e.g., via AAV-PHP.eB) are injected into the C9orf72G₄C₂ 300× heterozygous mice at postnatal day 0 by intracerebroventricular injection. Mice are sacrificed at selected time points post-injection, RNA is isolated from selected tissues, and RT-qPCR for C9orf72 transcripts is performed to assess reduction of transcription from exon 1A accompanied by reduction in intron-containing transcripts.

### Materials and Methods

ES cell culture, gRNA electroporations into ES cells. ES cells were grown on cell culture plates coated with gelatin (0.1% in water, Stem Cell Technologies) containing a monolayer of irradiated primary mouse embryonic fibroblasts (Produced in-house). Dulbecco's Minimal Essential Medium (DMEM, Gibco) with high glucose, 0.1 mM nonessential amino acids, 1 mM sodium pyruvate, 0.1 mM 2-mercaptoethanol, 2 mM L-glutamine, penicillin and streptomycin (50 μg/mL, Gibco), 15% FBS (Gemini Bio), 1200 U/mL LIF (Life Technologies) was used to grow and maintain ES cells at 37° C., 5% CO₂, and atmospheric O₂ level and passaged as needed.

Electroporations of gRNAs and Sp.dCas9 (SpCas9 with 2 inactivating mutations (D10A & H840A) that render the enzyme incapable of inducing double strand breaks; IDT #1081067) into ES cells was done with nucleofector 4D (Lonza) with the CP-105 program as per manufacturer instructions. Electroporation conditions for each type of experiment is as follows.

1 gRNA against the mouse exon 1a promoter region and 3 gRNAs against human exon 1a promoter region (FIGS. 5 and 6). For introducing 1 or 3 gRNAs directed against the promoter region for either mouse or human promoters respectively for C9orf72 exon 1A, and Sp.dCas9, into mouse ES cells (FIGS. 5 and 6), 31.25 pmol of dCas9 per 125 pmol of each sgRNA was mixed to form a complex with a total volume of 7.5 μL. The complexes were then mixed with 92.5 μL of P3 buffer (Lonza). This 100 μL of RNP complexes+P3 buffer was used to resuspend a cell pellet containing 2 million ES cells (this was an ES cell clone that harbored 300 G₄C₂ repeats). Electroporation was done with nucleofector 4D (Lonza) with the CP-105 program. Subsequently, cells were seeded at a density of 500,000 cells. The cells were harvested 24 h after the electroporation and processed for RNA extraction as described below.

4 gRNAs against the G₄C₂ repeats (all together) (FIGS. 7 and 8). For introducing all 4 gRNAs directed against the G₄C₂ repeats and Sp.dCas9 into mouse ES cells (FIGS. 7 and 8), 31.25 pmol of dCas9 per 125 pmol of each sgRNA was mixed to form a complex with a total volume of 10 μL. The complexes were then mixed with 90 ul of P3 buffer (Lonza). This 100 μL of RNP complexes+P3 buffer was used to resuspend a cell pellet containing 2 million ES cells (this was an ES cell clone that harbored 300 G₄C₂ repeats). Electroporation was done with nucleofector 4D (Lonza) with the CP-105 program. Subsequently, cells were seeded at a density of 500,000 cells. The cells were harvested 24 h after the electroporation and processed for RNA extraction as described below.

Reverse transcription-quantitative PCR (RT-qPCR) (FIGS. 6 and 8). We purified RNA from mouse ES cells with the RNeasy plus mini kit (Qiagen, Cat No: 74136) as per the manufacturer's instructions, followed by genomic DNA removal with the Turbo DNA-free kit (ThermoFisher Scientific, #AM1907). We diluted the purified RNA samples to a concentration of 20 ng/μl and performed RT-qPCR in a one-step reaction with QuantiNova Probe RT-PCR kit (Qiagen) according to the manufacturer's recommendations. The RT-qPCR contained 2 μL of RNA and an 8 μL of a mixture containing RT-PCR Master mix, ROX dye, RT-mix, and custom-designed primer-probe mix. The custom TaqMan assays (See Table below) were obtained from IDT and the reference assay for Drosha mRNA from ThermoFisher (cat. #Mm01310009_ml). We performed the qRT-PCR reactions in quadruplicate on a ViiA™ 7 Real-Time PCR Detection System (ThermoFisher) with the RT reaction at 45° C. for 10 min followed by PCR cycling (95° C. 10 min, 45 cycles of 95° C. 5 s, 60° C. 30 s) in an optical 384-well plate. We determined ΔCt values between the target RNA assays and the Drosha mRNA reference and calculated relative RNA abundance by the 2^{-ΔΔCt} method. A lower Ct and ΔCt value indicates higher amounts of target RNA detected.

TABLE 6
List of RT-qPCR TAQMAN ® assays used in the study.

| Assay Name | Primer/ Probe | Sequence | SEQ ID NO |
|---|---|---|---|
| Exon 1A-Exon 2 | Forward Primer | GAGCAGGTGTGGGTTTAGGA | 122 |
| | Reverse Primer | CCAGGTCTCACTGCATTCCA | 123 |
| | Probc | ATTGCAAGCGTTCGGATAATGTGAGA | 124 |
| Exon 1B-Exon 2 | Forward Primer | GGTGGCGAGTGGCTATTG | 125 |
| | Reverse Primer | GATAGTCGACATCCCTGCATC | 126 |
| | Probe | AAGCGTTCGGATAATGTGAGACCTGG | 127 |
| Exon 1A-Intron | Forward Primer | AAGAGCAGGTGTGGGTTTAG | 128 |
| | Reverse Primer | CGGTTGTTTCCCTCCTTGT | 129 |
| | Probe | TCTCACAGTACTCGCTGAGGGTGA | 130 |
| Intron (Adjacent | Forward Primer | CCCACTACTTGCTCTCACAG | 131 |
| to G4C2 repeats) | Reverse Primer | TACAGGCTGCGGTTGTTT | 132 |
| | Probe | ACTCGCTGAGGGTGAACAAGAAA | 133 |

Cloning C9orf72 G₄C₂ constructs with human promoter (FIG. 3). Previously, we generated a targeting vector that contains the mouse promoter region and either 3 or 250 G₄C₂ repeats. To swap the promoter with human sequence, we digested the 3× targeting vector using invitro CRISPR/Cas9 reaction using recombinant Cas9 protein and an Alt-R sgRNA (IDT) corresponding to the sequence: GCATTTTTGATGAAGCAAGA (SEQ ID NO: 134) and then using BbVCI restriction enzyme (NEB). A synthetic double stranded DNA fragment containing a ˜20 bp mouse homology arm and a 3 kb human promoter region was generated (GBLOCK™ Integrated DNA Technologies, IDT) and cloned into the digested targeting vector described above using Nebuilder HiFi DNA assembly kit (NEB) as per the manufacturer's instructions. Once the targeting vector containing the human promoter and a 3×G₄C₂ repeat was generated, this vector was digested with BbVCI and PspXI restriction enzymes to remove the 3 G₄C₂ repeats. The original 250 G₄C₂ repeats, with the mouse promoter was also subjected to restriction digestion using BbVCI and PspXI restriction enzymes and a fragment containing the 250 G₄C₂ repeats was recovered. This fragment was ligated into the above mentioned 3 G₄C₂ repeat containing vector followed by transformation of ELECTROMAX™ DH10β competent cells (Invitrogen #18290015) and culturing at 25° C. to reduce recombination events that collapse the G₄C₂ repeat expansion. Correctly recombined bacterial clones were expanded in LB broth plus carbenicillin (100 μg/mL; Sigma Aldrich), and the plasmids were purified with the EndoFree Plasmid maxi-prep kit (Zymo Research).

We claim:

1. A method of repressing transcription from a C9orf72 exon 1A transcription start site or repressing transcription of sense or antisense transcripts that comprise the hexanucleotide repeat expansion sequence in a C9orf72 gene in a cell, comprising:
(a) contacting the C9orf72 gene with a first CRISPR/Cas complex comprising a nuclease-inactive Cas protein and a first guide RNA comprising a first DNA-targeting segment that targets a first guide RNA target sequence upstream of or proximate to the C9orf72 exon 1A transcription start site, wherein the first CRISPR/Cas complex binds to the first guide RNA target sequence; and/or
(b) contacting the C9orf72 gene with a second CRISPR/Cas complex comprising the nuclease-inactive Cas protein and a second guide RNA comprising a second DNA-targeting segment that targets a second guide RNA target sequence within a C9orf72 hexanucleotide repeat expansion sequence between the first non-coding endogenous exon and exon 2 of the C9orf72 gene, wherein the second CRISPR/Cas complex binds to the second guide RNA target sequence.

2. The method of claim 1, wherein the method comprises option (a).

3. The method of claim 1, wherein the method comprises option (b).

4. The method of claim 1, wherein the method comprises options (a) and (b).

5. The method of any preceding claim, wherein option (a) comprises contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site, and/or
wherein option (b) comprises contacting the C9orf72 gene with at least two CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene.

6. The method of any preceding claim, wherein option (a) comprises contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence upstream of or proximate to the C9orf72 exon 1A transcription start site, and/or
wherein option (b) comprises contacting the C9orf72 gene with at least three CRISPR/Cas complexes, wherein each CRISPR/Cas complex comprises the nuclease-inactive Cas protein and a different guide RNA, wherein each different guide RNA targets a different guide RNA target sequence within the C9orf72 hexanucleotide repeat expansion sequence between the first non-coding endogenous exon and exon 2 of the C9orf72 gene.

7. The method of any preceding claim, wherein the C9orf72 hexanucleotide repeat expansion sequence has more than 30, more than 100, more than 200, more than 300, more than 400, or more than 500 repeats of the hexanucleotide sequence G₄C₂.

8. The method of any preceding claim, wherein the first guide RNA target sequence is within 250, within 225, within 200, within 175, within 150, within 125, within 100, within 75, or within 50 nucleotides of the C9orf72 exon 1A transcription start site.

9. The method of any preceding claim, wherein the first guide RNA target sequence is within 100, within 75, or within 50 nucleotides of the C9orf72 exon 1A transcription start site.

10. The method of any preceding claim, wherein the nuclease-inactive Cas protein is not fused to a heterologous transcriptional repressor domain, and the guide RNA is not linked to a heterologous transcriptional repressor domain.

11. The method of any preceding claim, wherein the binding reduces or abolishes expression of transcripts that initiate at C9orf72 exon 1A.

12. The method of any preceding claim, wherein the binding reduces or abolishes expression of transcripts that initiate at C9orf72 exon 1A but does not reduce or abolish expression of transcripts that initiate at C9orf72 exon 1B.

13. The method of any preceding claim, wherein the binding reduces or abolishes expression of C9orf72 hexanucleotide-repeat-containing transcripts.

14. The method of any preceding claim, wherein the binding reduces or abolishes expression of C9orf72 hexanucleotide-repeat-containing transcripts but does not reduce or abolish expression of transcripts that initiate at C9orf72 exon 1B.

15. The method of any preceding claim, wherein the binding reduces or abolishes expression of both sense and antisense C9orf72 hexanucleotide-repeat-containing transcripts.

16. The method of any preceding claim, wherein the binding reduces or abolishes expression of both sense and antisense C9orf72 hexanucleotide-repeat-containing transcripts but does not reduce or abolish expression of transcripts that initiate at C9orf72 exon 1B.

17. The method of any preceding claim, wherein the method comprises:
(a) introducing the nuclease-inactive Cas protein or a nucleic acid encoding the nuclease-inactive Cas protein and the first guide RNA or one or more DNAs encoding the first guide RNA into the cell; and/or
(b) introducing the nuclease-inactive Cas protein or the nucleic acid encoding the nuclease-inactive Cas protein and the second guide RNA or one or more DNAs encoding the second guide RNA into the cell.

18. The method of any preceding claim, wherein the first guide RNA is a single guide RNA (sgRNA).

19. The method of any preceding claim, wherein the nuclease-inactive Cas protein is a nuclease-inactive Cas9 protein.

20. The method of claim 19, wherein the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

21. The method of claim 19, wherein the nuclease-inactive Cas protein is derived from a Streptococcus pyogenes Cas9 protein.

22. The method of any preceding claim, wherein the nucleic acid encoding the nuclease-inactive Cas protein is codon-optimized for expression in a mammalian cell or a human cell.

23. The method of any preceding claim, wherein the method comprises:
(a) introducing the first guide RNA in the form of RNA, optionally wherein the first guide RNA comprises at least one modification; and/or
(b) introducing the second guide RNA in the form of RNA, optionally wherein the second guide RNA comprises at least one modification.

24. The method of claim 23, wherein the at least one modification comprises a 2′-O-methyl-modified nucleotide and/or a phosphorothioate bond between nucleotides.

25. The method of any preceding claim, wherein the method comprises introducing the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification.

26. The method of any one of claims 1-22, wherein the method comprises:
(a) introducing the nucleic acid encoding the nuclease-inactive Cas protein and the one or more DNAs encoding the first guide RNA, wherein the nucleic acid encoding the nuclease-inactive Cas protein comprises DNA; and/or
(b) introducing the nucleic acid encoding the nuclease-inactive Cas protein and the one or more DNAs encoding the second guide RNA, wherein the nucleic acid encoding the nuclease-inactive Cas protein comprises DNA.

27. The method of claim 26, wherein:
(a) the DNA encoding the nuclease-inactive Cas protein and the one or more DNAs encoding the first guide RNA are in one or more vectors; and/or
(b) the DNA encoding the nuclease-inactive Cas protein and the one or more DNAs encoding the second guide RNA are in one or more vectors.

28. The method of claim 27, wherein the one or more vectors are one or more viral vectors.

29. The method of claim 28, wherein the one or more viral vectors are one or more adeno-associated virus (AAV) vectors.

30. The method of any preceding claim, wherein:
(a) the nuclease-inactive Cas protein or the nucleic acid encoding the nuclease-inactive Cas protein and the first guide RNA or the one or more DNAs encoding the first guide RNA are associated with a lipid nanoparticle; and/or
(b) the nuclease-inactive Cas protein or the nucleic acid encoding the nuclease-inactive Cas protein and the second guide RNA or the one or more DNAs encoding the second guide RNA are associated with a lipid nanoparticle.

31. The method of any preceding claim, wherein:
(I) the first DNA-targeting segment comprises at least 17, at least 18, at least 19, or at least 20 contiguous nucleotides of the sequence set forth in any one of SEQ ID NOS: 72-111 and 113; and/or
(II) the first DNA-targeting segment is at least 90% or at least 95% identical to the sequence set forth in any one of SEQ ID NOS: 72-111 and 113; and/or
(III) the first guide RNA target sequence comprises at least 17, at least 18, at least 19, or at least 20 contiguous nucleotides of the sequence set forth in any one of SEQ ID NOS: 32-71 and 112.

32. The method of any preceding claim, wherein:
(I) the first DNA-targeting segment comprises at least 17, at least 18, at least 19, or at least 20 contiguous nucleotides of the sequence set forth in any one of SEQ ID NOS: 93-95; and/or
(II) the first DNA-targeting segment is at least 90% or at least 95% identical to the sequence set forth in any one of SEQ ID NOS: 93-95; and/or
(III) the first guide RNA target sequence comprises at least 17, at least 18, at least 19, or at least 20 contiguous nucleotides of the sequence set forth in any one of SEQ ID NOS: 53-55.

33. The method of any one of claims 1-31, wherein:
(I) the first DNA-targeting segment comprises at least 17, at least 18, at least 19, or at least 20 contiguous nucleotides of the sequence set forth in SEQ ID NO: 74; and/or
(II) the first DNA-targeting segment is at least 90% or at least 95% identical to the sequence set forth in SEQ ID NO: 74; and/or
(III) the first guide RNA target sequence comprises at least 17, at least 18, at least 19, or at least 20 contiguous nucleotides of the sequence set forth in SEQ ID NO: 34.

34. The method of any preceding claim, wherein:
(I) the second DNA-targeting segment comprises at least 17, at least 18, at least 19, or at least 20 contiguous nucleotides of the sequence set forth in any one of SEQ ID NOS: 118-121; and/or
(II) the second DNA-targeting segment is at least 90% or at least 95% identical to the sequence set forth in any one of SEQ ID NOS: 118-121; and/or
(III) the second guide RNA target sequence comprises at least 17, at least 18, at least 19, or at least 20 contiguous nucleotides of the sequence set forth in any one of SEQ ID NOS: 114-117.

35. The method of any preceding claim, wherein the cell is a neuron, optionally wherein the neuron is a motor neuron.

36. The method of any preceding claim, wherein the cell is in vitro or ex vivo.

37. The method of any one of claims 1-35, wherein the cell is in a subject in vivo, optionally wherein the subject is a human.

38. The method of claim 37, wherein the cell is a neuron in the brain of the subject.

39. The method of claim 37 or 38, wherein the subject has or is at risk for developing a C9orf72 hexanucleotide repeat expansion associated disease.

40. The method of claim 39, wherein the C9orf72 hexanucleotide repeat expansion associated disease is amyotrophic lateral sclerosis (ALS) or frontotemporal dementia (FTD).

41. The method of any one of claims 37-40, wherein:
(a) the nuclease-inactive Cas protein or a nucleic acid encoding the nuclease-inactive Cas protein and the first guide RNA or one or more DNAs encoding the first guide RNA are administered to the subject by intracerebroventricular injection, intracranial injection, or intrathecal injection; and/or
(b) the nuclease-inactive Cas protein or a nucleic acid encoding the nuclease-inactive Cas protein and the second guide RNA or one or more DNAs encoding the second guide RNA are administered to the subject by intracerebroventricular injection, intracranial injection, or intrathecal injection.

42. The method of any preceding claim, wherein the cell is a mammalian cell, and the C9orf72 gene is a mammalian C9orf72 gene.

43. The method of any preceding claim, wherein the cell is a human cell.

44. The method of any one of claims 1-42, wherein the cell is a mouse cell.

45. The method of any preceding claim, wherein the C9orf72 gene comprises a human C9orf72 promoter.

46. The method of any preceding claim, wherein the C9orf72 gene is a human C9orf72 gene or a humanized C9orf72 gene.

47. A method of repressing transcription from a C9orf72 exon 1A transcription start site or repressing transcription of sense or antisense transcripts that comprise the hexanucleotide repeat expansion sequence in a C9orf72 gene in a subject, comprising:
(a) administering to the subject a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or
(b) administering to the subject the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence.

48. A method of preventing, treating, or ameliorating at least one symptom or indication of a C9orf72 hexanucleotide repeat expansion associated disease, comprising:
(a) administering to a subject in need thereof a first pharmaceutical composition comprising a therapeutically effective amount of a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or
(b) administering to the subject in need thereof a second pharmaceutical composition comprising a therapeutically effective amount of the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence.

49. The method of claim 48, wherein the C9orf72 hexanucleotide repeat expansion associated disease is amyotrophic lateral sclerosis (ALS) or frontotemporal dementia (FTD).

50. The method of any one of claims 47-49, wherein the method comprises option (a).

51. The method of any one of claims 47-49, wherein the method comprises option (b).

52. The method of any one of claims 47-49, wherein the method comprises options (a) and (b).

53. The method of any one of claims 47-52, wherein option (a) comprises administering to the subject at least two guide RNAs, wherein each guide RNA guide RNA targets a different guide RNA target sequence upstream of or proximate to the C9orf72 exon 1A transcription start site, and/or
wherein option (b) comprises administering to the subject at least two guide RNAs, wherein each guide RNA guide RNA targets a different guide RNA target sequence within the C9orf72 hexanucleotide repeat expansion sequence between the first non-coding endogenous exon and exon 2 of the C9orf72 gene.

54. The method of any one of claims 47-53, wherein option (a) comprises administering to the subject at least three guide RNAs, wherein each guide RNA guide RNA targets a different guide RNA target sequence upstream of or proximate to the C9orf72 exon 1A transcription start site, and/or
wherein option (b) comprises administering to the subject at least three guide RNAs, wherein each guide RNA guide RNA targets a different guide RNA target sequence within the C9orf72 hexanucleotide repeat expansion sequence between the first non-coding endogenous exon and exon 2 of the C9orf72 gene.

55. The method of any one of claims 47-54, wherein the C9orf72 hexanucleotide repeat expansion sequence has more than 30, more than 100, more than 200, more than 300, more than 400, or more than 500 repeats of the hexanucleotide sequence G₄C₂.

56. The method of any one of claims 47-55, wherein the administering is intracerebroventricular injection, intracranial injection, or intrathecal injection.

57. The method of any one of claims 47-56, wherein the first guide RNA target sequence is within 250, within 225, within 200, within 175, within 150, within 125, within 100, within 75, or within 50 nucleotides of the C9orf72 exon 1A transcription start site.

58. The method of any one of claims 47-57, wherein the first guide RNA target sequence is within 100, within 75, or within 50 nucleotides of the C9orf72 exon 1A transcription start site.

59. The method of any one of claims 47-58, wherein the nuclease-inactive Cas protein is not fused to a heterologous transcriptional repressor domain, and the guide RNA is not linked to a heterologous transcriptional repressor domain.

60. The method of any one of claims 47-59, wherein the binding reduces or abolishes expression of transcripts that initiate at C9orf72 exon 1A.

61. The method of any one of claims 47-60, wherein the binding reduces or abolishes expression of transcripts that initiate at C9orf72 exon 1A but does not reduce or abolish expression of transcripts that initiate at C9orf72 exon 1B.

62. The method of any one of claims 47-61, wherein the binding reduces or abolishes expression of C9orf72 hexanucleotide-repeat-containing transcripts.

63. The method of any one of claims 47-62, wherein the binding reduces or abolishes expression of C9orf72 hexanucleotide-repeat-containing transcripts but does not reduce or abolish expression of transcripts that initiate at C9orf72 exon 1B.

64. The method of any one of claims 47-63, wherein the binding reduces or abolishes expression of both sense and antisense C9orf72 hexanucleotide-repeat-containing transcripts.

65. The method of any one of claims 47-64, wherein the binding reduces or abolishes expression of both sense and antisense C9orf72 hexanucleotide-repeat-containing transcripts but does not reduce or abolish expression of transcripts that initiate at C9orf72 exon 1B.

66. The method of any one of claims 47-65, wherein the first guide RNA is a single guide RNA (sgRNA).

67. The method of any one of claims 47-66, wherein the nuclease-inactive Cas protein is a nuclease-inactive Cas9 protein.

68. The method of claim 67, wherein the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

69. The method of claim 67, wherein the nuclease-inactive Cas protein is derived from a Streptococcus pyogenes Cas9 protein.

70. The method of any one of claims 47-69, wherein the nucleic acid encoding the nuclease-inactive Cas protein is codon-optimized for expression in a mammalian cell or a human cell.

71. The method of any one of claims 47-70, wherein the method comprises:
(a) administering the first guide RNA in the form of RNA, optionally wherein the first guide RNA comprises at least one modification; and/or
(b) administering the second guide RNA in the form of RNA, optionally wherein the second guide RNA comprises at least one modification.

72. The method of claim 71, wherein the at least one modification comprises a 2′-O-methyl-modified nucleotide and/or a phosphorothioate bond between nucleotides.

73. The method of any one of claims 47-72, wherein the method comprises administering the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification.

74. The method of any one of claims 47-70, wherein the method comprises:
(a) administering the nucleic acid encoding the nuclease-inactive Cas protein and the one or more DNAs encoding the first guide RNA, wherein the nucleic acid encoding the nuclease-inactive Cas protein comprises DNA; and/or
(b) administering the nucleic acid encoding the nuclease-inactive Cas protein and the one or more DNAs encoding the second guide RNA, wherein the nucleic acid encoding the nuclease-inactive Cas protein comprises DNA.

75. The method of claim 74, wherein:
(a) the DNA encoding the nuclease-inactive Cas protein and the one or more DNAs encoding the first guide RNA are in one or more vectors; and/or
(b) the DNA encoding the nuclease-inactive Cas protein and the one or more DNAs encoding the second guide RNA are in one or more vectors.

76. The method of claim 75, wherein the one or more vectors are one or more viral vectors.

77. The method of claim 76, wherein the one or more viral vectors are one or more adeno-associated virus (AAV) vectors.

78. The method of any one of claims 47-77, wherein:
(a) the nuclease-inactive Cas protein or the nucleic acid encoding the nuclease-inactive Cas protein and the first guide RNA or the one or more DNAs encoding the first guide RNA are associated with a lipid nanoparticle; and/or
(b) the nuclease-inactive Cas protein or the nucleic acid encoding the nuclease-inactive Cas protein and the second guide RNA or the one or more DNAs encoding the second guide RNA are associated with a lipid nanoparticle.

79. The method of any one of claims 47-78, wherein:
(I) the first DNA-targeting segment comprises at least 17, at least 18, at least 19, or at least 20 contiguous nucleotides of the sequence set forth in any one of SEQ ID NOS: 72-111 and 113; and/or
(II) the first DNA-targeting segment is at least 90% or at least 95% identical to the sequence set forth in any one of SEQ ID NOS: 72-111 and 113; and/or
(III) the first guide RNA target sequence comprises at least 17, at least 18, at least 19, or at least 20 contiguous nucleotides of the sequence set forth in any one of SEQ ID NOS: 32-71 and 112.

80. The method of any one of claims 47-79, wherein:
(I) the first DNA-targeting segment comprises at least 17, at least 18, at least 19, or at least 20 contiguous nucleotides of the sequence set forth in any one of SEQ ID NOS: 93-95; and/or
(II) the first DNA-targeting segment is at least 90% or at least 95% identical to the sequence set forth in any one of SEQ ID NOS: 93-95; and/or
(III) the first guide RNA target sequence comprises at least 17, at least 18, at least 19, or at least 20 contiguous nucleotides of the sequence set forth in any one of SEQ ID NOS: 53-55.

81. The method of any one of claims 47-79, wherein:
(I) the first DNA-targeting segment comprises at least 17, at least 18, at least 19, or at least 20 contiguous nucleotides of the sequence set forth in SEQ ID NO: 74; and/or
(II) the first DNA-targeting segment is at least 90% or at least 95% identical to the sequence set forth in SEQ ID NO: 74; and/or
(III) the first guide RNA target sequence comprises at least 17, at least 18, at least 19, or at least 20 contiguous nucleotides of the sequence set forth in SEQ ID NO: 34.

82. The method of any one of claims 47-81, wherein:
(I) the second DNA-targeting segment comprises at least 17, at least 18, at least 19, or at least 20 contiguous nucleotides of the sequence set forth in any one of SEQ ID NOS: 118-121; and/or
(II) the second DNA-targeting segment is at least 90% or at least 95% identical to the sequence set forth in any one of SEQ ID NOS: 118-121; and/or
(III) the second guide RNA target sequence comprises at least 17, at least 18, at least 19, or at least 20 contiguous nucleotides of the sequence set forth in any one of SEQ ID NOS: 114-117.

83. The method of any one of claims 47-82, wherein the binding is in neurons in the subject, optionally wherein the neurons are motor neurons.

84. The method of claim 83, wherein the neurons are in the brain of the subject.

85. The method of any one of claims 47-84, wherein the subject is a mammalian subject, and the C9orf72 gene is a mammalian C9orf72 gene.

86. The method of any one of claims 47-85, wherein the subject is a human subject.

87. The method of any one of claims 47-85, wherein the subject is a mouse subject.

88. The method of any one of claims 47-87, wherein the C9orf72 gene comprises a human C9orf72 promoter.

89. The method of any one of claims 47-88, wherein the C9orf72 gene is a human C9orf72 gene or a humanized C9orf72 gene.

90. A CRISPR/Cas system comprising:
(a) a nuclease-inactive Cas protein or a nucleic acid encoding the nuclease-inactive Cas protein and a first guide RNA or one or more DNAs encoding the first guide RNA, wherein the first guide RNA comprises a first DNA-targeting segment that targets a first guide RNA target sequence upstream of or proximate to the C9orf72 exon 1A transcription start site, wherein the first guide RNA and the nuclease-inactive Cas protein form a first CRISPR/Cas complex that binds to the first guide RNA target sequence; and/or
(b) the nuclease-inactive Cas protein or the nucleic acid encoding the nuclease-inactive Cas protein and a second guide RNA or one or more DNAs encoding the second guide RNA, wherein the second guide RNA comprises a second DNA-targeting segment that targets a second guide RNA target sequence within a C9orf72 hexanucleotide repeat expansion sequence between the first non-coding endogenous exon and exon 2 of the C9orf72 gene, wherein the second guide RNA and the nuclease-inactive Cas protein form a second CRISPR/Cas complex that binds to the second guide RNA target sequence.

91. The CRISPR/Cas system of claim 90, wherein the CRISPR/Cas system comprises option (a).

92. The CRISPR/Cas system of claim 90, wherein the CRISPR/Cas system comprises option (b).

93. The CRISPR/Cas system of claim 90, wherein the CRISPR/Cas system comprises options (a) and (b).

94. The CRISPR/Cas system of any one of claims 90-93, wherein option (a) comprises at least two guide RNAs, wherein each guide RNA targets a different guide RNA target sequence upstream of or proximate to the C9orf72 exon 1A transcription start site, and/or
wherein option (b) comprises at least two guide RNAs, wherein each guide RNA targets a different guide RNA target sequence within the C9orf72 hexanucleotide repeat expansion sequence between the first non-coding endogenous exon and exon 2 of the C9orf72 gene.

95. The CRISPR/Cas system of any one of claims 90-94, wherein option (a) comprises at least three guide RNAs, wherein each guide RNA targets a different guide RNA target sequence upstream of or proximate to the C9orf72 exon 1A transcription start site, and/or
wherein option (b) comprises at least three guide RNAs, wherein each guide RNA targets a different guide RNA target sequence within the C9orf72 hexanucleotide repeat expansion sequence between the first non-coding endogenous exon and exon 2 of the C9orf72 gene.

96. The CRISPR/Cas system of any one of claims 90-95, wherein the first guide RNA target sequence is within 250, within 225, within 200, within 175, within 150, within 125, within 100, within 75, or within 50 nucleotides of the C9orf72 exon 1A transcription start site.

97. The CRISPR/Cas system of any one of claims 90-96, wherein the first guide RNA target sequence is within 100, within 75, or within 50 nucleotides of the C9orf72 exon 1A transcription start site.

98. The CRISPR/Cas system of any one of claims 90-97, wherein the nuclease-inactive Cas protein is not fused to a heterologous transcriptional repressor domain, and the guide RNA is not linked to a heterologous transcriptional repressor domain.

99. The CRISPR/Cas system of any one of claims 90-98, wherein the binding reduces or abolishes expression of transcripts that initiate at C9orf72 exon 1A.

100. The CRISPR/Cas system of any one of claims 90-99, wherein the binding reduces or abolishes expression of transcripts that initiate at C9orf72 exon 1A but does not reduce or abolish expression of transcripts that initiate at C9orf72 exon 1B.

101. The CRISPR/Cas system of any one of claims 90-100, wherein the binding reduces or abolishes expression of C9orf72 hexanucleotide-repeat-containing transcripts.

102. The CRISPR/Cas system of any one of claims 90-101, wherein the binding reduces or abolishes expression of C9orf72 hexanucleotide-repeat-containing transcripts but does not reduce or abolish expression of transcripts that initiate at C9orf72 exon 1B.

103. The CRISPR/Cas system of any one of claims 90-102, wherein the binding reduces or abolishes expression of both sense and antisense C9orf72 hexanucleotide-repeat-containing transcripts.

104. The CRISPR/Cas system of any one of claims 90-103, wherein the binding reduces or abolishes expression of both sense and antisense C9orf72 hexanucleotide-repeat-containing transcripts but does not reduce or abolish expression of transcripts that initiate at C9orf72 exon 1B.

105. The CRISPR/Cas system of any one of claims 90-104, wherein the first guide RNA is a single guide RNA (sgRNA).

106. The CRISPR/Cas system of any one of claims 90-105, wherein the nuclease-inactive Cas protein is a nuclease-inactive Cas9 protein.

107. The CRISPR/Cas system of claim 106, wherein the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

108. The CRISPR/Cas system of claim 106, wherein the nuclease-inactive Cas protein is derived from a Streptococcus pyogenes Cas9 protein.

109. The CRISPR/Cas system of any one of claims 90-108, wherein the nucleic acid encoding the nuclease-inactive Cas protein is codon-optimized for expression in a mammalian cell or a human cell.

110. The CRISPR/Cas system of any one of claims 90-109, wherein the CRISPR/Cas system comprises:
(a) the first guide RNA in the form of RNA, optionally wherein the first guide RNA comprises at least one modification; and/or
(b) the second guide RNA in the form of RNA, optionally wherein the second guide RNA comprises at least one modification.

111. The CRISPR/Cas system of claim 110, wherein the at least one modification comprises a 2′-O-methyl-modified nucleotide and/or a phosphorothioate bond between nucleotides.

112. The CRISPR/Cas system of any one of claims 90-111, wherein the CRISPR/Cas system comprises the nucleic acid encoding the nuclease-inactive Cas protein, wherein the nucleic acid comprises an mRNA encoding the nuclease-inactive Cas protein, optionally wherein the mRNA encoding the nuclease-inactive Cas protein comprises at least one modification.

113. The CRISPR/Cas system of any one of claims 90-109, wherein the CRISPR/Cas system comprises:
(a) the nucleic acid encoding the nuclease-inactive Cas protein and the one or more DNAs encoding the first guide RNA, wherein the nucleic acid encoding the nuclease-inactive Cas protein comprises DNA; and/or
(b) the nucleic acid encoding the nuclease-inactive Cas protein and the one or more DNAs encoding the second guide RNA, wherein the nucleic acid encoding the nuclease-inactive Cas protein comprises DNA.

114. The CRISPR/Cas system of claim 113, wherein:
(a) the DNA encoding the nuclease-inactive Cas protein and the one or more DNAs encoding the first guide RNA are in one or more vectors; and/or
(b) the DNA encoding the nuclease-inactive Cas protein and the one or more DNAs encoding the second guide RNA are in one or more vectors.

115. The CRISPR/Cas system of claim 114, wherein the one or more vectors are one or more viral vectors.

116. The CRISPR/Cas system of claim 115, wherein the one or more viral vectors are one or more adeno-associated virus (AAV) vectors.

117. The CRISPR/Cas system of any one of claims 90-116, wherein:
(a) the nuclease-inactive Cas protein or the nucleic acid encoding the nuclease-inactive Cas protein and the first guide RNA or the one or more DNAs encoding the first guide RNA are associated with a lipid nanoparticle; and/or
(b) the nuclease-inactive Cas protein or the nucleic acid encoding the nuclease-inactive Cas protein and the second guide RNA or the one or more DNAs encoding the second guide RNA are associated with a lipid nanoparticle.

118. The CRISPR/Cas system of any one of claims 90-117, wherein:
(I) the first DNA-targeting segment comprises at least 17, at least 18, at least 19, or at least 20 contiguous nucleotides of the sequence set forth in any one of SEQ ID NOS: 72-111 and 113; and/or
(II) the first DNA-targeting segment is at least 90% or at least 95% identical to the sequence set forth in any one of SEQ ID NOS: 72-111 and 113; and/or
(III) the first guide RNA target sequence comprises at least 17, at least 18, at least 19, or at least 20 contiguous nucleotides of the sequence set forth in any one of SEQ ID NOS: 32-71 and 112.

119. The CRISPR/Cas system of any one of claims 90-118, wherein:
(I) the first DNA-targeting segment comprises at least 17, at least 18, at least 19, or at least 20 contiguous nucleotides of the sequence set forth in any one of SEQ ID NOS: 93-95; and/or
(II) the first DNA-targeting segment is at least 90% or at least 95% identical to the sequence set forth in any one of SEQ ID NOS: 93-95; and/or
(III) the first guide RNA target sequence comprises at least 17, at least 18, at least 19, or at least 20 contiguous nucleotides of the sequence set forth in any one of SEQ ID NOS: 53-55.

120. The CRISPR/Cas system of any one of claims 90-118, wherein:
(I) the first DNA-targeting segment comprises at least 17, at least 18, at least 19, or at least 20 contiguous nucleotides of the sequence set forth in SEQ ID NO: 74; and/or
(II) the first DNA-targeting segment is at least 90% or at least 95% identical to the sequence set forth in SEQ ID NO: 74; and/or
(III) the first guide RNA target sequence comprises at least 17, at least 18, at least 19, or at least 20 contiguous nucleotides of the sequence set forth in SEQ ID NO: 34.

121. The CRISPR/Cas system of any one of claims 90-120, wherein:
(I) the second DNA-targeting segment comprises at least 17, at least 18, at least 19, or at least 20 contiguous nucleotides of the sequence set forth in any one of SEQ ID NOS: 118-121; and/or
(II) the second DNA-targeting segment is at least 90% or at least 95% identical to the sequence set forth in any one of SEQ ID NOS: 118-121; and/or
(III) the second guide RNA target sequence comprises at least 17, at least 18, at least 19, or at least 20 contiguous nucleotides of the sequence set forth in any one of SEQ ID NOS: 114-117.

122. The CRISPR/Cas system of any one of claims 90-121, wherein the C9orf72 gene is a mammalian C9orf72 gene.

123. The CRISPR/Cas system of any one of claims 90-122, wherein the C9orf72 gene comprises a human C9orf72 promoter.

124. The CRISPR/Cas system of any one of claims 90-123, wherein the C9orf72 gene is a human C9orf72 gene or a humanized C9orf72 gene.

125. A pharmaceutical composition comprising the CRISPR/Cas system of any one of claims 90-124 and a pharmaceutically acceptable carrier.

126. A composition comprising a guide RNA or one or more DNAs encoding the guide RNA, wherein the guide RNA comprises a DNA-targeting segment that targets a guide RNA target sequence in a C9orf72 gene, wherein the guide RNA target sequence is within a C9orf72 hexanucleotide repeat expansion sequence between the first non-coding endogenous exon and exon 2 of the C9orf72 gene, and wherein the guide RNA can bind to a nuclease-inactive Cas protein and target the nuclease-inactive Cas protein to the guide RNA target sequence.

127. The composition of claim 126, wherein the binding of the Cas protein to the guide RNA target sequence reduces or abolishes expression of transcripts that initiate at C9orf72 exon 1A.

128. The composition of claim 126 or 127, wherein binding of the Cas protein to the guide RNA target sequence reduces or abolishes expression of transcripts that initiate at C9orf72 exon 1A but does not reduce or abolish expression of transcripts that initiate at C9orf72 exon 1B.

129. The composition of any one of claims 126-128, wherein binding of the Cas protein to the guide RNA target sequence reduces or abolishes expression of C9orf72 hexanucleotide-repeat-containing transcripts.

130. The composition of any one of claims 126-129, wherein binding of the Cas protein to the guide RNA target sequence reduces or abolishes expression of C9orf72 hexanucleotide-repeat-containing transcripts but does not reduce or abolish expression of transcripts that initiate at C9orf72 exon 1B.

131. The composition of any one of claims 126-130, wherein binding of the Cas protein to the guide RNA target sequence reduces or abolishes expression of both sense and antisense C9orf72 hexanucleotide-repeat-containing transcripts.

132. The composition of any one of claims 126-131, wherein binding of the Cas protein to the guide RNA target sequence reduces or abolishes expression of both sense and antisense C9orf72 hexanucleotide-repeat-containing transcripts but does not reduce or abolish expression of transcripts that initiate at C9orf72 exon 1B.

133. The composition of any one of claims 126-132, wherein the guide RNA is a single guide RNA (sgRNA).

134. The composition of any one of claims 126-133, wherein the nuclease-inactive Cas protein is a nuclease-inactive Cas9 protein.

135. The composition of claim 134, wherein the nuclease-inactive Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

136. The composition of claim 134, wherein the nuclease-inactive Cas protein is derived from a Streptococcus pyogenes Cas9 protein.

137. The composition of any one of claims 126-136, wherein the CRISPR/Cas system comprises the guide RNA in the form of RNA, optionally wherein the guide RNA comprises at least one modification.

138. The composition of claim 137, wherein the at least one modification comprises a 2′-O-methyl-modified nucleotide and/or a phosphorothioate bond between nucleotides.

139. The composition of claim 138, wherein the one or more DNAs encoding the guide RNA are in one or more vectors.

140. The composition of claim 139, wherein the one or more vectors are one or more viral vectors.

141. The composition of claim 140, wherein the one or more viral vectors are one or more adeno-associated virus (AAV) vectors.

142. The composition of any one of claims 126-141, wherein the guide RNA or the one or more DNAs encoding the guide RNA are associated with a lipid nanoparticle.

143. The composition of any one of claims 126-142, wherein:
(I) the DNA-targeting segment comprises at least 17, at least 18, at least 19, or at least 20 contiguous nucleotides of the sequence set forth in any one of SEQ ID NOS: 118-121; and/or
(II) the DNA-targeting segment is at least 90% or at least 95% identical to the sequence set forth in any one of SEQ ID NOS: 118-121; and/or
(III) the guide RNA target sequence comprises at least 17, at least 18, at least 19, or at least 20 contiguous nucleotides of the sequence set forth in any one of SEQ ID NOS: 114-117.

144. The composition of any one of claims 126-143, wherein the C9orf72 gene is a mammalian C9orf72 gene.

145. The composition of any one of claims 126-144, wherein the C9orf72 gene comprises a human C9orf72 promoter.

146. The composition of any one of claims 126-145, wherein the C9orf72 gene is a human C9orf72 gene or a humanized C9orf72 gene.

147. A pharmaceutical composition comprising the composition of any one of claims 126-146 and a pharmaceutically acceptable carrier.
